(19) United States
(12) Patent Application Publication
(10) Pub. No.: US 2003/0147861 A1
(43) Pub. Date: Aug. 7, 2003

(54) Compounds and methods for the modulation of immune responses

(75) Inventors: James D. Watson, Auckland (NZ); Paul L.J. Tan, Bondi Junction (AU); Nevin Abernethy, Auckland (NZ)

Correspondence Address: SPECKMAN LAW GROUP, 1501 WESTERN AVE, SUITE 100, SEATTLE, WA, 98101, US

(73) Assignee: Genesis Research and Development Corporation Limited, Auckland (NZ)

(21) Appl. No.: 10/205,979

(22) Filed: Jul. 25, 2002

Related U.S. Application Data
(60) Provisional application No. 60/308,446, filed on Jul. 26, 2001.

(51) Int. Cl. A61K 45/00; A61K 39/04

(52) U.S. Cl. 424/93.7; 424/248.1

(57) ABSTRACT

Methods and compositions for the modification of immune response by modulating of the Notch signaling pathway are provided, together with methods for the treatment of disorders characterized by the presence of an unwanted immune response. Such compositions comprise components derived from Mycobacteria, such as Mycobacterium vaccae.

### REFERENCE TO RELATED APPLICATIONS

This application claims priority to U.S. Provisional Patent Application No. 60/308,446, filed Jul. 26, 2001.

### TECHNICAL FIELD

The present invention relates generally to the modification of immune system responses. In particular, the invention is related to compositions and methods for the modification of T cell responses by means of modulating the expression of molecules involved in the Notch signaling and Toll-like receptor signaling pathways, and for the treatment of disorders in which these pathways play a role.

### BACKGROUND OF THE INVENTION

Certain disorders, such as autoimmune disorders (for example, multiple sclerosis, rheumatoid artliritis, Type I diabetes mellitus, psoriasis, systemic lupus erythematosus and scleroderma), allergic disorders and graft rejection, are characterized by the presence of an undesirable and abnormal immune response to either a self or foreign antigen. In such disorders, suppression of the immune response, such as by induction of a negative T cell response or induction of tolerance towards the antigen, is thus highly desirable.

Recognition of an antigen by naive CD4+ T cells in the peripheral immune system can lead to either activation of an immune response against the antigen or to the induction of tolerance wherein T cells become refractory to further stimulation with antigen. The choice between immune activation and tolerance is controlled by signals delivered by antigen presenting cells (APCs) at the time of initial presentation of the antigen by the APC. Once tolerance has been induced in a small number of T cells (known as T regulatory, or Tr cells), this tolerance can be transmitted to other T cells, thereby actively suppressing an immune response to the antigen. This phenomenon is known as “infectious tolerance” or “linked suppression”. The induction of tolerance in naïve T cells by Tr cells is believed to occur either through direct cell-cell interactions or by secretion of inhibitory cytokines, such as IL-4, IL-10 and TGF-beta.

The Notch signaling pathway is known to play an important role in regulating cell growth and differentiation. Proteins of the Notch family are large transmembrane proteins which function as receptors and that were originally identified in Drosophila. In mammals, four different Notch receptors (known as Notch 1-4) and at last five different ligands (Jagged-1, Jagged-2, Delta-like 1, Delta-like 3 and Delta-like 4) have been identified, with Jagged being the mammalian homologue of the Serrate ligand identified in Drosophila. The nucleotide sequences of the human Notch and Delta genes, and the amino acid sequences of their encoded proteins are disclosed in International Patent Publication WO 92/19734. The Notch signaling pathway is highly conserved from D. melanogaster through to humans, indicating the importance of this pathway in regulating cell growth and differentiation.

Hoyne et al. (Immunology 100:281-288, 2000), have demonstrated that expression of Notch ligands on T cells and APCs can lead to the development of T-cell tolerance. More specifically, Hoyne et al. propose that recognition of antigen on APCs which also express Notch ligands induces naive T cells to differentiate into Tr cells. The activated Tr cell then expresses a Notch ligand (such as Delta) at its surface. This in turn engages Notch on neighboring naïve T cells, thereby directly influencing the growth of naive T cells, and leading to linked suppression. Modification of the Notch signaling pathway, for example by modulation of expression of a Notch receptor or ligand, may thus be employed to modify or suppress an undesirable immune response in a disorder by inducing tolerance to a particular antigen.

Interaction of Notch with its ligands has been shown to trigger the release of the intracellular domain of Notch (N^{IC}) which in turn binds to either Deltex or CBF-1, a sequence-specific DNA transcription factor also known as RBP-Jκ. By binding to Deltex or CBF1, N^{IC} can alter the capacity of these molecules to regulate transcription of various genes. Activation of Deltex can result in repression of the basic helix-loop-helix protein E47, which is a regulator of B and T cell development and, more specifically, is involved in the determination of B versus T cell fate. Binding of N^{IC} to CBF-1 activates transcription of the Hairy Enhancer of Split (HES) family of proteins. Disruption of HES has severe consequences on the immune system, including defects in thymic development. Specifically, HES-1 has been shown to repress CD4 expression and to affect early thymocyte precursors. Binding of N^{IC} to CBF-1 also increases expression of NF-κB2, whose activity has been associated with protection from apoptosis in lymphoid tissue (Oswald et at. Mol. Cell. Biol. 18:207-2088, 1998). Notch expression has been shown to rescue cells from apoptosis (Deftos et al. Immunity 9:777-786, 1998; Jehn et al. J. Immunol. 162:635-638, 1999; and Shelly et al. J. Cell. Biochem. 73:164-175, 1999), and it has been suggested that Notch expression may affect cell fate through direct regulation of apoptosis (Osborne et al. Immunity 11:653-663, 1999). More recently, the proteins Lunatic Fringe, Manic Fringe and Radical Fringe have been shown to act as potent regulators of Notch-1 expression (see, for example, Koch et al. (Immunity 15:225-236, 2001)). These proteins may regulate Notch-1 activation in lymphoid precursors to ensure that T and C cells develop in different tissues. Other molecules known to involved in Notch signaling include Numb, which inhibits Notch signaling; presenilinl, which is a Notch signaling regulator; HERP1 and 2, which are both downstream signaling targets; and the basic helix-loop-helix (bHLH) transcription factor HASH1 which has recently been shown to be degraded by activated Notch (Sriuranpong et at, Mol. Cell. Biol. 22:3129-39, 2002).

### SUMMARY OF THE INVENTION

Briefly stated, the present invention provides compositions and methods for suppression and modification of immune responses by modulating the expression of molecules involved in the Notch signaling and Toll-like receptor signaling pathways, together with compositions and methods for the treatment of disorders characterized by an unwanted immune response, such as autoimmune disorders, allergic disorders and graft rejection.

In one aspect, the present invention provides methods for modulating the expression of Notch ligands on antigen present cells, such as dendritic cells and macrophages, by contacting the antigen presenting cells with a composition described herein. In a further aspect, methods for modulating Notch and/or Toll-like receptor signaling in a population of cells, either in vivo or in vitro, are provided, such methods comprising contacting the cells with a composition of the present invention. In yet another aspect, methods are provided for modifying an immune response to an antigen in a subject, and for stimulating infectious tolerance to an antigen in a subject, such methods comprising administering to the subject an effective amount of one or more of the compositions described herein.

In related aspects, the present invention provides methods for the treatment of a disorder characterized by an unwanted immune response in a patient, such methods comprising administering to the patient a composition of the present invention. In certain embodiments, the disorder is selected from the group consisting of autoimmune disorders (including, but not limited to, multiple sclerosis, rheumatoid arthritis, Type I diabetes mellitus, psoriasis, systemic lupus erythematosus and scleroderma), allergic diseases and graft rejection.

As discussed above, the Notch signaling pathway is also involved in apoptotic cell death mechanisms. Specifically, when Notch is expressed, cells are protected from apoptotic cell death. According to additional aspects of the present invention, methods are provided for treatment of a disorder characterized by undesired apoptotic cell death, and for treatment of a disorder characterized by undesired cell proliferation, such methods comprising modulating the Notch signaling pathway by administering a composition described herein.

In certain embodiments, the inventive methods comprise administering a composition, wherein the composition comprises inactivated mycobacterial cells or a derivative thereof, such as delipidated and deglycolipidated mycobacterial cells. In preferred embodiments, the delipidated and deglycolipidated cells are prepared from M. vaccae, M. tuberculosis or M. smegmatis. In further embodiments, the inventive methods comprise administering a composition comprising peptidoglycan.

In other embodiments, the compositions employed in the inventive methods comprise a derivative of delipidated and deglycolipidated mycobacterial cells, the derivative being selected from the group consisting of: delipidated and deglycolipidated mycobacterial cells that have been treated by acid hydrolysis; delipidated and deglycolipidated mycobacterial cells that have been treated by alkaline hydrolysis; delipidated and deglycolipidated mycobacterial cells that have been treated with periodic acid; delipidated and deglycolipidated mycobacterial cells that have been treated with Proteinase K; and delipidated and deglycolipidated mycobacterial cells that have been treated by anhydrous hydrofluoric acid hydrolysis. In specific embodiments, such derivatives are prepared from M. vaccae, M. tuberculosis or M. smegmatis. The derivatives of delipidated and deglycolipidated M. vaccae preferably contain galactose in an amount less than 9.7% of total carbohydrate, more preferably less than 5% of total carbohydrate, and most preferably less than 3.5% total carbohydrate. In certain embodiments, the derivatives of delipidated and deglycolipidated M. vaccae contain glucosamine in an amount greater than 3.7% of total carbohydrate, preferably greater than 5% total carbohydrate and more preferably greater than 7.5% total carbohydrate.

In yet another aspect, the compositions disclosed herein comprise an isolated polypeptide derived from Mycobacterium vaccae or an isolated polynucleotide encoding such a polypeptide, such polypeptides comprising at least an immunogenic portion of an M. vaccae antigen, or a variant thereof. In specific embodiments, such polypeptides comprise an amino acid sequence selected from the group consisting of: (a) sequences recited in SEQ ID NO: 27-52; (b) sequences encoded by any one of SEQ ID NO: 1-26; (c) sequences having at least about 75% identity to a sequence recited in SEQ ID NO: 27-52; (d) sequences having at least about 90% identity to a sequence recited in SEQ ID NO: 27-52, as measured using alignments produced by the computer algorithm BLASTP as described below.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS

FIG. 1 illustrates the re-suspension of DD-M. vaccae and DD-M. vaccae-KOH.

FIG. 2 shows the suppression by DD-M. vaccae (Q1) and the DD-M. vaccae derivatives Q2 (DD-M. vaccae-KOH), Q3 (DD-M. vaccae-acid), Q4 (DD-M. vaccae-periodate), Q6 (DD-M. vaccae-KOH-periodate), P5 (DD-M. vaccae-KOH-acid) and P6 (DD-M. vaccae-KOH-periodate) of ovalbumin-induced airway eosinophilia in mice vaccinated intranasally with these compounds. Control mice received PBS.

FIG. 3 illustrates the effect of immunization with DD-M. vaccae on airway eosinophilia when administered either one day prior, at the time of, or one day after challenge with OVA.

FIG. 4 shows the stimulation of IL-10 production in THP-1 cells by derivatives of DD-M. vaccae.

FIG. 5 illustrates the effect of immunization with DD-M. vaccae, DD-M. tuberculosis and DD-M. smegmatis on airway eosinophilia.

FIG. 6 illustrates TNF-α production by human PBMC and non-adherent cells stimulated with DD-M. vaccae.

FIGS. 7A and 7B illustrate IL-10 and IFN-γ production, respectively, by human PBMC and non-adherent cells stimulated with DD-M. vaccae.

FIGS. 8A-C illustrate the stimulation of CD69 expression on αβT cells, γδT cells and NK cells, respectively, by the M. vaccae protein GV23, the Th1-inducing adjuvants MPL/TDM/CWS and CpG ODN, and the Th2-inducing adjuvants aluminium hydroxide and cholera toxin.

FIGS. 9A-D illustrate the effect of heat-killed M. vaccae, DD-M. vaccae and M. vaccae recombinant proteins on the production of IL-1β, TNF-α, IL-12 and IFN-γ, respectively, by human PBMC.

FIGS. 10A-C illustrate the effects of varying concentrations of the recombinant M. vaccae proteins GV-23 and GV-45 on the production of IL-1β, TNF-α and IL-12, respectively, by human PBMC.

FIGS. 11A-D illustrate the stimulation of IL-1β, TNF-α, IL-12 and IFN-γ production, respectively, in human PBMC by the M. vaccae protein GV23, the Th1-inducing adjuvants MPL/TDM/CWS and CpG ODN, and the Th2-inducing adjuvants aluminium hydroxide and cholera toxin.

FIGS. 12A-C illustrate the effects of varying concentrations of the recombinant M. vaccae proteins GV-23 and GV-45 on the expression of CD40, CD80 and CD86, respectively, by dendritic cells.

FIG. 13 illustrates the enhancement of dendritic cell mixed lymphocyte reaction by the recombinant M. vaccae protein GV-23.

FIG. 14 illustrates real-time PCR analysis demonstrating that treatment of mice with AVAC produced increases in expression of Notch receptors, ligands, and downstream targets.

FIG. 15A-C illustrate the effect of heat-killed M. vaccae, DD-M. vaccae (referred to in the Figure as PVAC) and AVAC, respectively, on the expression of genes involved in Notch signaling in THP-1 cells.

FIG. 16 illustrates the effect of intranasal administration of AVAC and DD-M. vaccae (referred to in the Figure as PVAC) in mice on expression of genes involved in Notch signaling.

FIG. 17 illustrates the effect of intraperitoneal administration of AVAC in mice on the expression of cytokines and genes involved in Notch signaling.

FIG. 18 shows the production of IL-12p40 by THP-1 cells in response to increasing concentrations of M. vaccae derivatives.

FIG. 19 shows the production of IL-12p40, IL-23p19 and IL-12p35 mRNA in THP-1 cells in response to AVAC, DD-M. vaccae, heat-killed M. vaccae and M. vaccae glycolipids.

FIGS. 20A-C illustrate the production of IL-12p40 by THP-1 cells cultured with antibodies to Toll-like receptors and either heat-killed M. vaccae, DD-M. vaccae or AVAC, respectively.

FIGS. 21A-C illustrate the production of TNF-alpha by THP-1 cells cultured with antibodies to Toll-like receptors and either heat-killed M. vaccae, DD-M. vaccae or LPS, respectively.

FIG. 22 shows the production of IL-10 by THP-1 cells cultured with antibodies to Toll-like receptors and heat-killed M. vaccae.

FIG. 23 illustrates the production of IL-10 by THP-1 cells cultured with MAP kinase inhibitors and AVAC.

### DETAILED DESCRIPTION OF THE INVENTION

As noted above, the present invention is generally directed to compositions and methods for modulating immune responses by modification of the Notch signaling pathway. The inventive compositions and methods may thus be employed in the treatment of disorders characterized by the presence of an unwanted immune response to either a self antigen or a foreign antigen, such as autoimmune disorders, allergic disorders and graft rejection. Examples of autoimmune disorders include multiple sclerosis, rheumatoid arthritis, Type I diabetes mellitus, psoriasis, systemic lupus erythematosus and scleroderma. Examples of allergic disorders include atopic dermatitis, eczema, asthma, allergic rhinitis, contact allergies and hypersensitivities.

Certain pathogens, such as M. tuberculosis, as well as certain cancers, are effectively contained by an immune attack directed by CD4⁺ T cells, known as cell-mediated immunity. Other pathogens, such as poliovirus, also require antibodies, produced by B cells, for containment. These different classes of immune attack (T cell or B cell) are controlled by different subpopulations of CD4⁺ T cells, commonly referred to as Th1 and Th2 cells. The two types of Th cell subsets have been well characterized and are defined by the cytokines they release upon activation. The Th1 subset secretes IL-2, IFN-γ and tumor necrosis factor, and mediates macrophage activation and delayed-type hypersensitivity response. The Th2 subset releases IL-4, IL-5, IL-6 and IL-10, which stimulate B cell activation. The Th1 and Th2 subsets are mutually inhibiting, so that IL-4 inhibits Th1-type responses, and IFN-γ inhibits Th2-type responses.

Amplification of Th1-type immune responses is central to a reversal of disease in many disorders. IL-12 has been shown to up-regulate Th1 responses, while IL-10 has been shown to down-regulate Th2 responses. The inventors have discovered that both delipidated and deglycolipidated M. vaccae cells (referred to herein as DD-M. vaccae) and delipidated and deglycolipidated M. vaccae cells further treated by acid hydrolysis (referred to herein as AVAC) have pronounced immunoregulatory effects on both Th2 and Th1 cells. For example, as detailed below, the inventors have demonstrated the efficacy of both DD-M. vaccae and AVAC in the treatment of asthma employing a mouse model. These compositions are believed to be effective in the treatment of diseases such as asthma due to their ability to down-regulate asthma-inducing Th2 immune responses, as shown by the reduction in total IgE and antigen-specific IgE and IgG1.

In clinical trials on the effectiveness of DD-M. vaccae in the treatment psoriasis, local injections of DD-M. vaccae were observed to lead to clearance of distant skin lesions, demonstrating the involvement of a systemic mechanism of action. No in vitro proliferation in response to DD-M. vaccae stimulation was observed in peripheral blood mononuclear cells (PBMC) taken from DD-M. vaccae-treated patients, thereby indicating the lack of a specific T cell response to DD-M. vaccae. Experimental data is presented, below, in Example 9.

As described below, DD-M. vaccae is ingested by cells of the THP-1 human monocytic cell line and stimulates these cells to secrete IL-10 and IL-12. DD-M. vaccae stimulates blood-derived human dendritic cells to upregulate the expression of CD40, CD80 and CD86 costimulatory molecules in vitro. T cell and NK cells show increased expression of the CD69 activation molecule when exposed to DD-M. vaccae, and the antigen presenting function of mouse dendritic cells is enhanced when bone marrow derived dendritic cells are pre-tested with DD-M. vaccae in vitro. Taken together, these results indicate that DD-M. vaccae modifies the response to endogenous psoriatic antigen by affecting antigen presentation.

As the clinical effects of DD-M. vaccae on psoriasis are systemic and distant psoriatic lesions are cleared following local injection of DD-M. vaccae, it is likely that DD-M. vaccae is transported to the lymph nodes where it influences APCs and T cells. Alternatively, either APCs or both APCs and regulatory T cells activated by DD-M. vaccae migrate to lymph nodes and the circulation. The APCs then terminate the generation of pathologic T cells, and T cells down regulating psoriatic pathology proliferate either in the lymph nodes or systemically.

While the expression of costimulatory molecules (CD40, CD80 and CD86) by antigen presenting cells is required for antigen presentation, and the secretion of IL-10 is likely to be important in regulating T cell responses, other molecules are required to generate T regulatory cells as a population distinct from effector T helper cells. As discussed above, the Notch ligand family of molecules is known to determine fate of cells during T cell development. Genes and molecules that determine differentiation of T cells during development are likely to influence the differentiation of T cell subsets during an immune response. The fact that DD-M. vaccae and its derivatives do not suppress antigen presentation and stimulate cytokine production, indicates that they may be successfully employed to modify an immune response to an antigen at the time of antigen presentation, and may also suppress an immune response that has occurred after antigen presentation.

As detailed below, the inventors have demonstrated that a derivative of DD-M. vaccae, namely AVAC, induces production of Notch ligands on antigen presenting cells (APCs). Recognition of an antigen on these up-regulated APCs, induces naïve T cells to differentiate into regulatory T (Tr) cells and to express a Notch ligand. The Notch ligand on the Tr cells in turn interacts with Notch on neighboring naïve T cells, leading to the induction of infectious tolerance to the antigen. The inventors have also demonstrated that AVAC, DD-M. vaccae, inactivated M. vaccae and M. vaccae glycolipids modulate expression of various genes involved in Notch signaling both in vitro and in vivo, as well as genes involved in Toll-like receptor and cytokine signaling.

While not wishing to be bound by theory, the inventors believe, based on the experimental results presented below, that interaction of M. vaccae, DD-M. vaccae and AVAC with human myelomonocytic THP-1 cells is mediated in part by the specific binding of M. vaccae-derived cell wall components, principally peptidoglycan, to the extracellular domain of Toll-like receptor 2 (TLR2), one of several pathogen receptors expressed by these cells. Ligation of TLR2 then initiates an intracellular signaling cascade leading to the transcription of cytokine genes and translation of cytokine mRNA into biologically active protein. The cytokines so elicited have a variety of biological effects, including the capacity to influence expression of: genes involved in Notch signaling; TLR signaling genes themselves; and other inflammation-associated genes such as that for the calcium-binding protein MRP8.

As described in detail below, the inventors have demonstrated that M. vaccae derivatives up- or down-regulate expression of genes encoding Notch receptors, Notch ligands, downstream targets of Notch signaling, and Notch-active glycosyltransferases in human THP-1 cells. It is believed that this occurs partly via the actions of cytokines and cytokine signaling pathway mediators induced by Toll-like receptor (TLR) signaling, and partly via bona fide Notch signaling. As discussed above, Notch signaling occurs in cells expressing Notch receptors, and is initiated when Notch receptors are specifically ligated by Notch ligands. Although THP-1 cells express all of the Notch receptors and ligands described herein, it is likely that very little Notch signaling occurs in cultures of free-floating THP-1 cells in the absence of external stimuli. However, by ligating TLR2 on adjacent THP-1 cells, inactivated M. vaccae, DD-M. vaccae and AVAC bring THP-1 cells into very close contact with one another, thereby facilitating multiple productive interactions between Notch receptors and Notch ligands, which in turn leads to signal transduction in the Notch-bearing cell. Ligation of Notch receptor leads to proteolytic release of Notch intracellular domain (N^{IC}), the intracellular mediator responsible for entering the nucleus and, in co-operation with additional molecules, initiating transcription of: downstream Notch signaling genes such as HES1, Deltex and HERP; Notch receptor, Notch ligand, and Notch-active glycosyltransferase genes by one or more autocrine feedback loops; and other genes whose expression is influenced by Notch signaling (for example, Numb). Within this framework, recognition of M. vaccae derivatives by THP-1 cells is mediated by TLR2, and decision-making is mediated by both downstream products of TLR signaling (changes in expression of TLR and cytokine genes) and by Notch signaling.

As used herein the term “inactivated M. vaccae” refers to M. vaccae cells that have either been killed by means of heat, as detailed below in Example 1, or by exposure to radiation, such as ⁶⁰Cobalt at a dose of 2.5 megarads, or by any other inactivation technique. As used herein, the term “modified M. vaccae” includes delipidated M. vaccae cells, deglycolipidated M. vaccae cells, M. vaccae cells that have been both delipidated and deglycolipidated (DD-M. vaccae), and derivatives of delipidated and deglycolipidated M. vaccae cells. DD-M. vaccae may be prepared as described below in Example 1, with the preparation of derivatives of DD-M. vaccae being detailed below in Example 2. The preparation of delipidated and deglycolipidated M. tuberculosis (DD-M. tuberculosis) and M. smegmatis (DD-M. smegmatis) is described in Example 5, below. Derivatives of DD-M. tuberculosis and DD-M. smegmatis, such as acid-treated, alkali-treated, periodate-treated, proteinase K-treated, and/or hydrofluoric acid-treated derivatives, may be prepared using the procedures disclosed herein for the preparation of derivatives of DD-M. vaccae.

The derivatives of DD-M. vaccae preferably contain galactose in an amount less than 9.7% of total carbohydrate, more preferably less than 5% of total carbohydrate, and most preferably less than 3.5% total carbohydrate. In certain embodiments, the derivatives of DD-M. vaccae preferably contain glucosamine in an amount greater than 3.7% of total carbohydrate, more preferably greater than 5% total carbohydrate, and most preferably greater than 7.5% total carbohydrate. Derivatives prepared by treatment of DD-M. vaccae with alkali, such as DD-M. vaccae-KOH (also known as KVAC), have a reduced number of ester bonds linking mycolic acids to the arabinogalactan of the cell wall compared to DD-M. vaccae, and are thus depleted of mycolic acids. Derivatives prepared by treatment with acid, such as DD-M. vaccae-acid (also referred to as AVAC), have a reduced number of phosphodiester bonds attaching arabinogalactan sidechains to the peptidoglycan of the cell wall, and are therefore depleted of arabinogalactan. In addition, such derivatives are depleted of DNA. Derivatives prepared by treatment of DD-M. vaccae with periodate, such as DD-M. vaccae-periodate (also known as IVAC), have a reduced number of cis-diol-containing sugar residues compared to DD-M. vaccae and are depleted of arabinogalactan. Derivatives prepared by treatment of DD-M. vaccae with Proteinase K (such as the derivative referred to as EVAC) are depleted of proteins and peptides. Derivatives prepared by treatment with hydrofluoric acid, such as DD-M. vaccae-KOH treated with hydrofluoric acid (referred to as HVAC), are depleted of glycosidic bonds.

In certain embodiments, compositions that may be effectively employed in the inventive methods include polypeptides that comprise at least a functional portion of an M. vaccae antigen, or a variant thereof. As used herein, the term “polypeptide” encompasses amino acid chains of any length, including full length proteins (i.e., antigens), wherein the amino acid residues are linked by covalent peptide bonds. Thus, a polypeptide comprising a functional portion of an antigen may consist entirely of the functional portion, or may contain additional sequences. The additional sequences may be derived from the native M. vaccae antigen or may be heterologous.

A “functional portion” as used herein means a portion of an antigen that possesses an ability to modulate the expression of a protein involved in the Notch signaling pathway. The ability of an antigen, or a portion thereof, to modulate expression of a protein involved in the Notch signaling pathway may be determined as described below in Examples 11-14.

The term “polynucleotide(s),” as used herein, means a single or double-stranded polymer of deoxyribonucleotide or ribonucleotide bases and includes DNA and corresponding RNA molecules, including HnRNA and mRNA molecules, both sense and anti-sense strands, and comprehends cDNA, genomic DNA and recombinant DNA, as well as wholly or partially synthesized polynucleotides. An HnRNA molecule contains introns and corresponds to a DNA molecule in a generally one-to-one manner. An mRNA molecule corresponds to an HnRNA and DNA molecule from which the introns have been excised. A polynucleotide may consist of an entire gene, or any portion thereof. Operable anti-sense polynucleotides may comprise a fragment of the corresponding polynucleotide, and the definition of “polynucleotide” therefore includes all such operable anti-sense fragments. Antisense polynucleotides and techniques involving antisense polynucleotides are well known in the art and are described, for example, in Robinson-Benion et al., “Antisense techniques,” Methods in Enzymol. 254(23):363-375, 1995; and Kawasaki et al., Artific. Organs 20 (8):836-848, 1996.

As used herein, the term “variant” comprehends nucleotide or amino acid sequences different from the specifically identified sequences, wherein one or more nucleotides or amino acid residues is deleted, substituted, or added. Variants may be naturally occurring allelic variants, or non-naturally occurring variants, and include polynucleotides that encode identical amino acid sequences or essentially identical sequences differing by codon alterations that reflect the degeneracy of the genetic code. In addition to these “silent variations”, it is understood by those skilled in the art that conservative substitutions can be made by substituting particular amino acids with chemically similar amino acids without changing the function of the polypeptide (see e.g., Creighton, “Proteins”, W. H. Freeman and Company (1984).

Variant sequences (polynucleotide or polypeptide) preferably exhibit at least 75%, more preferably at least 90%, and most preferably at least 95% identity to a sequence of the present invention. The percentage identity is determined by aligning the two sequences to be compared as described below, determining the number of identical residues in the aligned portion, dividing that number by the total number of residues in the inventive (queried) sequence, and multiplying the result by 100. By way of example only, assume a queried polynucleotide having 220 nucleic acids has a hit to a polynucleotide sequence in the EMBL database having 520 nucleic acids over a stretch of 23 nucleotides in the alignment produced by the BLASTN algorithm using the default parameters as described below. The 23 nucleotide hit includes 21 identical nucleotides, one gap and one different nucleotide. The percentage identity of the queried polynucleotide to the hit in the EMBL database is thus 21/220 times 100, or 9.5%. The percentage identity of polypeptide sequences may be determined in a similar fashion.

Polynucleotide and polypeptide sequences may be aligned, and percentages of identical residues in a specified region may be determined against another polynucleotide or polypeptide sequence, using computer algorithms that are publicly available. Two exemplary algorithms for aligning and identifying the similarity of polynucleotide sequences are the BLASTN and FASTA algorithms. Polynucleotides may also be analyzed using the BLASTX algorithm, which compares the six-frame conceptual translation products of a nucleotide query sequence (both strands) against a protein sequence database. The percentage identity of polypeptide sequences may be examined using the BLASTP algorithm. The BLASTN, BLASTP and BLASTX algorithms are available on the NCBI anonymous FTP server under /blast/executables/ and are available from the National Center for Biotechnology Information (NCBI), National Library of Medicine, Building 38A, Room 8N805, Bethesda, Md. 20894, USA. The BLASTN algorithm Version 2.0.11 [Jan. 20, 2000], set to the parameters described below, is preferred for use in the determination of polynucleotide variants according to the present invention. The BLASTP algorithm, set to the parameters described below, is preferred for use in the determination of polypeptide variants according to the present invention. The use of the BLAST family of algorithms, including BLASTN, BLASTP and BLASTX, is described in the publication of Altschul, et al., Nucleic Acids Res. 25:3389-3402, 1997.

The FASTA and FASTX algorithms are available on the Internet, and from the University of Virginia by contacting the Vice Provost for Research, University of Virginia, P.O. Box 9025, Charlottesville, Va. 22906-9025, USA. The FASTA algorithm, set to the default parameters described in the documentation and distributed with the algorithm, may be used in the determination of polynucleotide variants. The readme files for FASTA and FASTX Version 1.0x that are distributed with the algorithms describe the use of the algorithms and describe the default parameters. The use of the FASTA and FASTX algorithms is described in Pearson and Lipman, Proc. Natl. Acad. Sci. USA 85:2444-2448, 1988; and Pearson, Methods in Enzymol. 183:63-98, 1990.

The following running parameters are preferred for determination of alignments and similarities using BLASTN that contribute to the E values and percentage identity for polynucleotides: Unix running command with the following default parameters: blastall -p blastn -d embldb -e 10 -G 0 -E 0 -r 1 -v 30 -b 30 -i queryseq -o results; and parameters are: -p Program Name [String]; -d Database [String]; -e Expectation value (E) [Real]; -G Cost to open a gap (zero invokes default behavior) [Integer]; -E Cost to extend a gap (zero invokes default behavior) [Integer]; -r Reward for a nucleotide match (blastn only) [Integer]; -v Number of one-line descriptions (V) [Integer]; -b Number of alignments to show (B) [Integer]; -i Query File [File In]; -o BLAST report Output File [File Out] Optional.

The following running parameters are preferred for determination of alignments and similarities using BLASTP that contribute to the E values and percentage identity of polypeptide sequences: blastall -p blastp -d swissprotdb -e 10 -G 0 -E 0 -v 30 -b 30 -i queryseq -o results; the parameters are: -p Program Name [String]; -d Database [String]; -e Expectation value (E) [Real]; -G Cost to open a gap (zero invokes default behavior) [Integer]; -E Cost to extend a gap (zero invokes default behavior) [Integer]; -v Number of one-line descriptions (v) [Integer]; -b Number of alignments to show (b) [Integer]; -I Query File [File In]; -o BLAST report Output File [File Out] Optional.

The “hits” to one or more database sequences by a queried sequence produced by BLASTN, BLASTP, FASTA, or a similar algorithm, align and identify similar portions of sequences. The hits are arranged in order of the degree of similarity and the length of sequence overlap. Hits to a database sequence generally represent an overlap over only a fraction of the sequence length of the queried sequence. The BLASTN, FASTA and BLASTP algorithms also produce “Expect” values for polynucleotide and polypeptide alignments. The Expect value (E) indicates the number of hits one can “expect” to see over a certain number of contiguous sequences by chance when searching a database of a certain size. The Expect value is used as a significance threshold for determining whether the hit to a database indicates true similarity. For example, an E value of 0.1 assigned to a polynucleotide hit is interpreted as meaning that in a database of the size of the EMBL database, one might expect to see 0.1 matches over the aligned portion of the sequence with a similar score simply by chance. By this criterion, the aligned and matched portions of the sequences then have a probability of 90% of being related. For sequences having an E value of 0.01 or less over aligned and matched portions, the probability of finding a match by chance in the EMBL database is 1% or less using the BLASTN algorithm. E values for polypeptide sequences may be determined in a similar fashion using various polypeptide databases, such as the SwissProt database.

According to one embodiment, “variant” polynucleotides and polypeptides, with reference to each of the polynucleotides and polypeptides of the present invention, preferably comprise sequences having the same number or fewer nucleic or amino acids than each of the polynucleotides or polypeptides of the present invention and producing an E value of 0.01 or less when compared to the polynucleotide or polypeptide of the present invention. That is, a variant polynucleotide or polypeptide is any sequence that has at least a 99% probability of being the same as the polynucleotide or polypeptide of the present invention, measured as having an E value of 0.01 or less using the BLASTN, FASTA or BLASTP algorithms set at the default parameters. According to a preferred embodiment, a variant polynucleotide is a sequence having the same number or fewer nucleic acids than a polynucleotide of the present invention that has at least a 99% probability of being the same as the polynucleotide of the present invention, measured as having an E value of 0.01 or less using the BLASTN algorithm set at the default parameters. Similarly, according to a preferred embodiment, a variant polypeptide is a sequence having the same number or fewer amino acids than a polypeptide of the present invention that has at least a 99% probability of being the same as the polypeptide of the present invention, measured as having an E value of 0.01 or less using the BLASTP algorithm set at the default parameters.

In addition to having a specified percentage identity to an inventive polynucleotide or polypeptide sequence, variant polynucleotides and polypeptides preferably have additional structure and/or functional features in common with the inventive polynucleotide or polypeptide. Polypeptides having a specified degree of identity to a polypeptide of the present invention share a high degree of similarity in their primary structure and have substantially similar functional properties. In addition to sharing a high degree of similarity in their primary structure to polynucleotides of the present invention, polynucleotides having a specified degree of identity to, or capable of hybridizing to, an inventive polynucleotide preferably have at least one of the following features: (i) they contain an open reading frame or partial open reading frame encoding a polypeptide having substantially the same functional properties as the polypeptide encoded by the inventive polynucleotide; or (ii) they contain identifiable domains in common.

In certain embodiments, variant polynucleotides hybridize to a polynucleotide of the present invention under stringent conditions. As used herein, “stringent conditions” refers to prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

The present invention also encompasses polynucleotides that differ from the disclosed sequences but that, as a consequence of the discrepancy of the genetic code, encode a polypeptide having similar enzymatic activity as a polypeptide encoded by a polynucleotide of the present invention. Thus, polynucleotides comprising sequences that differ from the polynucleotide sequences recited in SEQ ID NOS: 1-26 (or complements, reverse sequences, or reverse complements of those sequences) as a result of conservative substitutions are encompassed within the present invention. Additionally, polynucleotides comprising sequences that differ from the inventive polynucleotide sequences or complements, reverse complements, or reverse sequences as a result of deletions and/or insertions totaling less than 10% of the total sequence length are also contemplated by and encompassed within the present invention. Similarly, polypeptides comprising sequences that differ from the inventive polypeptide sequences as a result of amino acid substitutions, insertions, and/or deletions totalling less than 10% of the total sequence length are contemplated by and encompassed within the present invention, provided the variant polypeptide has similar activity to the inventive polypeptide.

A polypeptide described herein may be conjugated to a signal (or leader) sequence at the N-terminal end of the protein which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

In general, M. vaccae antigens, and polynucleotides encoding such antigens, may be prepared using any of a variety of procedures. For example, soluble antigens may be isolated from M. vaccae culture filtrate. Antigens may also be produced recombinantly by inserting a DNA sequence that encodes the antigen into an expression vector and expressing the antigen in an appropriate host. Any of a variety of expression vectors known to those of ordinary skill in the art may be employed. Expression may be achieved in any appropriate host cell that has been transformed or transfected with an expression vector containing a polynucleotide that encodes a recombinant polypeptide. Suitable host cells include prokaryotes, yeast and higher eukaryotic cells. Preferably, the host cells employed are E. coli, mycobacteria, insect, yeast or a mammalian cell line such as COS or CHO. The DNA sequences expressed in this manner may encode naturally occurring antigens, portions of naturally occurring antigens, or other variants thereof.

Polynucleotides encoding M. vaccae antigens may be obtained by screening an appropriate M. vaccae cDNA or genomic DNA library for DNA sequences that hybridize to degenerate oligonucleotides derived from amino acid sequences of isolated antigens. Suitable degenerate oligonucleotides may be designed and synthesized, and the screen may be performed as described, for example in Sambrook et al., Molecular Cloning: A Laboratory Manual, Cold Spring Harbor Laboratories, Cold Spring Harbor, N.Y., 1989. Polymerase chain reaction (PCR) may be employed to isolate a nucleic acid probe from genomic DNA, or a cDNA or genomic DNA library. The library screen may then be performed using the isolated probe. DNA molecules encoding M. vaccae antigens may also be isolated by screening an appropriate M. vaccae expression library with anti-sera (e.g., rabbit or monkey) raised specifically against M. vaccae antigens.

Regardless of the method of preparation, the antigens described herein have the ability to modify an immune response. More specifically, the antigens have the ability to effect the Notch signaling pathway by modulation of the expression of proteins involved in the Notch signaling pathway including, but not limited to, Notch or Notch ligands on APCs and/or T cells. The ability of an antigen to modulate the expression of proteins involved in the Notch signaling pathway may be determined as described below in Example 11-14.

Portions and other variants of M. vaccae antigens may be generated by synthetic or recombinant means. Synthetic polypeptides having fewer than about 100 amino acids, and generally fewer than about 50 amino acids, may be generated using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Perkin Elmer/Applied BioSystems, Inc. (Foster City, Calif.), and may be operated according to the manufacturer's instructions. Variants of a native antigen may be prepared using standard mutagenesis techniques, such as oligonucleotide-directed site-specific mutagenesis. Sections of the DNA sequence may also be removed using standard techniques to permit preparation of truncated polypeptides.

In general, regardless of the method of preparation, the polypeptides and polynucleotides disclosed herein are prepared in an isolated, substantially pure, form. Preferably, the polypeptides and polynucleotides are at least about 80% pure, more preferably at least about 90% pure and most preferably at least about 99% pure.

Alternatively, a composition of the present invention may contain DNA encoding one or more polypeptides as described above, such that the polypeptide is generated in situ. In such compositions, the DNA may be present within any of a variety of delivery systems known to those of ordinary skill in the art, including nucleic acid expression systems, bacterial and viral expression systems. Appropriate nucleic acid expression systems contain the necessary DNA sequences for expression in the patient (such as a suitable promoter and terminator signal). Bacterial delivery systems involve the administration of a bacterium (such as Bacillus-Calmette-Guerin) that expresses an immunogenic portion of the polypeptide on its cell surface. In a preferred embodiment, the DNA may be introduced using a viral expression system (e.g., vaccinia or other poxvirus, retrovirus, or adenovirus), which may involve the use of a non-pathogenic, or defective, replication competent virus. Techniques for incorporating DNA into such expression systems are well known in the art. The DNA may also be “naked,” as described, for example, in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

As noted above, the compositions describe herein may be employed for the treatment of disorders including autoimmune disorders, allergic disorders and graft rejection. When used in such methods, the compositions described herein may be administered by injection (e.g., intradermal, intramuscular, intravenous or subcutaneous), intranasally (e.g., by aspiration), orally or epicutaneously (applied topically onto skin). In one embodiment, the compositions are in a form suitable for delivery to the mucosal surfaces of the airways leading to or within the lungs. For example, the composition may be suspended in a liquid formulation for delivery to a patient in an aerosol form or by means of a nebulizer device.

For use in therapeutic methods, the compositions described herein may additionally contain a physiologically acceptable carrier. While any suitable carrier known to those of ordinary skill in the art may be employed in the compositions of this invention, the type of carrier will vary depending on the mode of administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed.

The preferred frequency of administration and effective dosage will vary from one individual to another. For both DD-M. vaccae and derivatives of DD-M. vaccae, the amount present in a dose preferably ranges from about 10 μg to about 1000 μg, more preferably from about 10 μg to about 100 μg. The number of doses may range from 1 to about 10 administered over a period of up to 12 months. In general, the amount of polypeptide present in a dose (or produced in situ by the DNA in a dose) ranges from about 1 pg to about 100 mg per kg of host, typically from about 10 pg to about 1 mg, and preferably from about 100 pg to about 1 μg. Suitable dose sizes will vary with the size of the patient, but will typically range from about 0.1 ml to about 5 ml.

The word “about,” when used in this application with reference to the amount of active component in a dose, contemplates a variance of up to 5% from the stated amount.

The following examples are offered by way of illustration and are not limiting.

### EXAMPLE 1

### Preparation of Delipidated and Deglycolipidated M. vaccae (DD-M. vaccae)

This example illustrates the processing of different constituents of M. vaccae and their immune modulating properties.

Heat-killed M. vaccae and M. vaccae Culture Filtrate

M. vaccae (American Type Culture Collection Number 15483) was cultured in sterile Medium 90 (yeast extract, 2.5 g/l; tryptone, 5 g/l; glucose 1 g/l) at 37° C. The cells were harvested by centrifugation, and transferred into sterile Middlebrook 7H9 medium (Difco Laboratories, Detroit, Mich.) with glucose at 37° C. for one day. The medium was then centrifuged to pellet the bacteria, and the culture filtrate removed. The bacterial pellet was resuspended in phosphate buffered saline at a concentration of 10 mg/ml, equivalent to 10¹⁰ M. vaccae organisms per ml. The cell suspension was then autoclaved for 15 min at 120° C. The culture filtrate was passaged through a 0.45 μm filter into sterile bottles.

Preparation of Delipidated and Deglycolipidated M. vaccae (DD-M. vaccae) and Compositional Analysis

To prepare delipidated M. vaccae, the autoclaved M. vaccae was pelleted by centrifugation, the pellet washed with water and collected again by centrifugation, and freeze-dried. An aliquot of this freeze-dried M. vaccae was set aside and referred to as lyophilised M. vaccae. When used in experiments it was resuspended in PBS to the desired concentration. Freeze-dried M. vaccae was treated with chloroform/methanol (2:1) for 60 min at room temperature to extract lipids, and the extraction was repeated once. The delipidated residue from the chloroform/methanol extraction was further treated with 50% ethanol to remove glycolipids by refluxing for two hours. The 50% ethanol extraction was repeated two times. The pooled 50% ethanol extracts were used as a source of M. vaccae glycolipids. The residue from the 50% ethanol extraction was freeze-dried and weighed. The amount of delipidated and deglycolipidated M. vaccae prepared was equivalent to 11.1% of the starting wet weight of M. vaccae used. For bioassay, the delipidated and deglycolipidated M. vaccae (DD-M. vaccae), was resuspended in phosphate-buffered saline by sonication, and sterilized by autoclaving.

The compositional analyses of heat-killed M. vaccae and DD-M. vaccae are presented in Table 1. Major changes are seen in the fatty acid composition and amino acid composition of DD-M. vaccae as compared to the insoluble fraction of heat-killed M. vaccae. The data presented in Table 1 show that the insoluble fraction of heat-killed M. vaccae contains 10% w/w of lipid, and the total amino acid content is 2750 nmoles/mg, or approximately 33% w/w. DD-M. vaccae contains 1.3% w/w of lipid and 4250 nmoles/mg amino acids, which is approximately 51% w/w.

TABLE 1
Compositional analyses of heat-killed
M. vaccae and DD-M. vaccae

| | |
|---|---|
| M. vaccae | DD-M. vaccae |

MONOSACCHARIDE COMPOSITION

| | | |
|---|---|---|
| sugar alditol | | |
| Inositol | 3.2% | 1.7% |
| Ribitol* | 1.7% | 0.4% |
| Arabinitol | 22.7% | 27.0% |
| Mannitol | 8.3% | 3.3% |
| Galactitol | 11.5% | 12.6% |
| Glucitol | 52.7% | 55.2% |

Fatty Acid Composition

| | | |
|---|---|---|
| Fatty acid | | |
| C14:0 | 3.9% | 10.0% |
| C16:0 | 21.1% | 7.3% |
| C16:1 | 14.0% | 3.3% |
| C18:0 | 4.0% | 1.5% |
| C18:1* | 1.2% | 2.7% |
| C18:1w9 | 20.6% | 3.1% |
| C18:1w7 | 12.5% | 5.9% |
| C22:0 | 12.1% | 43.0% |
| C24:1* | 6.5% | 22.9% |

Amino Acid Composition

| | | |
|---|---|---|
| nmoles/mg | | |
| ASP | 231 | 361 |
| THR | 170 | 266 |
| SER | 131 | 199 |
| GLU | 319 | 505 |
| PRO | 216 | 262 |
| GLY | 263 | 404 |
| ALA | 416 | 621 |
| CYS* | 24 | 26 |
| VAL | 172 | 272 |
| MET* | 72 | 94 |
| ILE | 104 | 171 |
| LEU | 209 | 340 |
| TYR | 39 | 75 |
| PHE | 76 | 132 |
| GlcNH2 | 5 | 6 |
| HIS | 44 | 77 |
| LYS | 108 | 167 |
| ARG | 147 | 272 |

The insoluble fraction of heat-killed M. vaccae contains 10% w/w of lipid, and DD-M. vaccae contains 1.3% w/w of lipid.
The total amino acid content of the insoluble fraction of heat-killed M. vaccae is 2750 nmoles/mg, or approximately 33% w/w. The total amino acid content of DD-M. vaccae is 4250 nmoles/mg, or approximately 51% w/w.

M. vaccae Glycolipids

The pooled 50% ethanol extracts described above were dried by rotary evaporation, redissolved in water, and freeze-dried. The amount of glycolipid recovered was 1.2% of the starting wet weight of M. vaccae used. For bioassay, the glycolipids were dissolved in phosphate-buffered saline.

### EXAMPLE 2

### Preparation and Characterization of Additional Derivatives of M. vaccae

Alkaline Hydrolysis of DD-M. vaccae

This procedure is intended to cleave linkages that are labile to alkaline lysis, such as the ester bonds linking mycolic acids to the arabinogalactan of the mycobacterial cell wall.

One gram of DD-M. vaccae, prepared as described in Example 1, was suspended in 20 ml of a 0.5% solution of potassium hydroxide (KOH) in ethanol. Other alkaline agents and solvents are well known in the art and may be used in the place of KOH and ethanol. The mixture was incubated at 37° C. with intermittent mixing for 48 hours. The solid residue was harvested by centrifugation, and washed twice with ethanol and once with diethyl ether. The product was air-dried overnight. The yield was 1.01 g (101%) of KOH-treated DD-M. vaccae, subsequently referred to as DD-M. vaccae-KOH (also known as KVAC). This derivative was found to be more soluble than the other derivatives of DD-M. vaccae disclosed herein.

Acid Hydrolysis of DD-M. vaccae

This procedure is intended to cleave acid-labile linkages, such as the phosphodiester bonds attaching the arabinogalactan sidechains to the peptidoglycan of the mycobacterial cell wall.

DD-M. vaccae or DD-M. vaccae-KOH (100 mg) was washed twice in 1 ml of 50 mM H₂SO₄ followed by resuspension and centrifugation. Other acids are well known in the art and may be used in place of sulphuric acid. For the acid hydrolysis step, the solid residue was resuspended in 1 ml of 50 mM H₂SO₄, and incubated at 60° C. for 72 hours. Following recovery of the solid residue by centrifugation, the acid was removed by washing the residue five times with water. The freeze-dried solid residue yielded 58.2 mg acid-treated DD-M. vaccae (DD-M. vaccae-acid; also known as AVAC) or 36.7 mg acid-treated DD-M. vaccae-KOH (DD-M. vaccae-KOH-acid).

Periodic Acid Cleavage of DD-M. vaccae

This procedure is intended to cleave cis-diol-containing sugar residues in DD-M. vaccae, such as the rhamnose residue near the attachment site of the arabinogalactan chains to the peptidoglycan backbone.

DD-M. vaccae or DD-M. vaccae-KOH (100 mg) was suspended in 1 ml of a solution of 1% periodic acid in 3% acetic acid, incubated for 1 hour at room temperature and the solid residue recovered by centrifugation. This periodic acid treatment was repeated three times. The solid residue was recovered by centrifugation, and incubated with 5 ml of 0.1 M sodium borohydride for one hour at room temperature. The resulting solid residue was recovered by centrifugation and the sodium borohydride treatment repeated. After centrifugation, the solid residue was washed four times with water and freeze-dried to give a yield of 62.8 mg DD-M. vaccae-periodate (also known as IVAC) or 61.0 mg DD-M. vaccae-KOH-periodate.

Resuspension of DD-M. vaccae and DD-M. vaccae-KOH

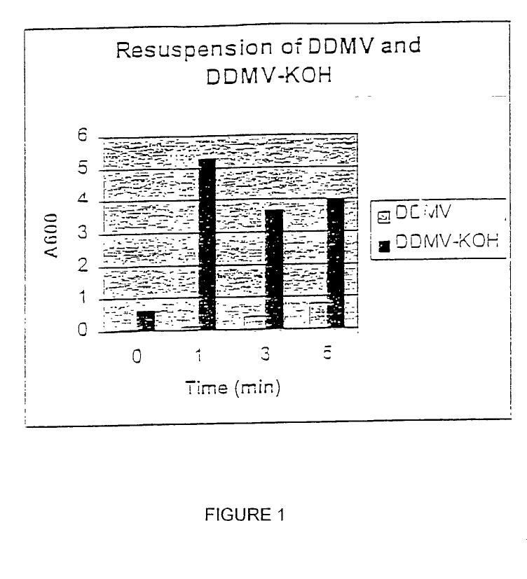

DD-M. vaccae and DD-M. vaccae-KOH (11 mg each) were suspended in phosphate-buffered saline (5.5 ml). Samples were sonicated with a Virtis probe sonicator for various times at room temperature (mini-probe, 15% output). Samples were then vortexed for sixty seconds and allowed to stand for five minutes to allow the sedimentation of large particles. The absorbance of the remaining suspension at 600 nm was measured. As shown in FIG. 1, DD-M. vaccae-KOH (referred to in FIG. 1 as DDMV-KOH) was fully resuspended after one minute's sonication, and further sonication produced no further increase in the absorbance. After five minutes sonication, the resuspension of DD-M. vaccae (referred to in FIG. 1 as DDMV) was still incomplete as estimated from the absorbance of the suspension. These results indicate that DD-M. vaccae-KOH is considerably more soluble than DD-M. vaccae.

Proteinase K Hydrolysis of DD-M. vaccae

This procedure is intended to digest proteins and peptides, while leaving most other materials intact.

One hundred milligrams of DD-M. vaccae, prepared as described in Example 1, was suspended in 9 ml water with sonication. Sodium dodecyl sulfate (SDS) was added to a final concentration of 1% w/v, and Proteinase K to a final concentration of 100 μg/ml w/v. The reaction mixture was incubated at 50° C. for 16 hours. The product was harvested by centrifugation, washed with phosphate-buffered saline and water, and lyophilized. The yield was 59 mg (59%) of Proteinase K-treated DD-M. vaccae, subsequently referred to as EVAC.

Hydrofluoric Acid Hydrolysis of KOH-treated DD-M. vaccae

This procedure is intended to cleave linkages that are labile to hydrolysis with anhydrous hydrofluoric acid, such as glycosidic bonds, while leaving most proteins intact.

One gram of DD-M. vaccae-KOH, prepared as described above, was suspended in 15 ml liquid hydrogen fluoride containing anisole as a free-radical scavenger. The mixture was incubated at 0° C. with mixing for one hour. The hydrogen fluoride (HF) was removed by distillation, and the solid residue was washed with diethyl ether to remove the anisole. The resulting product was extracted with water to yield water-soluble and water-insoluble fractions. The yield was 250 mg (25%) of water-soluble material, and 550 mg (55%) of water-insoluble HF-hydrolyzed KOH-treated DD-M. vaccae, subsequently referred to as HVAC.

Carbohydrate Compositional Analysis of DD-M. vaccae and DD-M. vaccae Derivatives

The carbohydrate composition of DD-M. vaccae and DD-M. vaccae derivatives was determined using standard techniques. The results are shown in Table 2, wherein DDMV represents DD-M. vaccae; DDMV-KOH represents DD-M. vaccae-KOH; DDMV-A represents DD-M. vaccae-acid; DDMV-I represents DD-M. vaccae-periodate; DDMV-KOH-A represents DD-M. vaccae-KOH-acid; and DDMV-KOH-I represents DD-M. vaccae-KOH-periodate.

TABLE 2
Carbohydrate Compositional Analysis of DD-M. vaccae and DD-M. vaccae Derivatives

| | | DDMV- | | | DDMV- | DDMV- |
|---|---|---|---|---|---|---|
| Carbohydrate | DDMV | KOH | DDMV-A | DDMV-I | KOH-A | KOH-I |
| Galactosamine | 26.6* | 29.2 | 14.9 | 37.7 | 0.3 | 3.9 |
| Glucosamine | 3.7 | 3.6 | 8.7 | 35.6 | 12.2 | 63.2 |
| Galactose | 9.7 | 9.2 | 0.7 | 3.4 | 0.0 | 0.0 |
| Glucose | 56.9 | 54.8 | 71.1 | 23.0 | 87.5 | 27.5 |
| Mannose | 3.2 | 3.2 | 4.7 | 0.4 | 0.02 | 5.5 |
| Fucose | Not detected | Not detected | Not detected | Not detected | Not detected | Not detected |

*All values in % of total carbohydrate

The results demonstrate that each of the DD-M. vaccae derivatives had a different carbohydrate content, as expected from the different effects of the acid, periodate or alkali treatment of the cells. In addition, DD-M. vaccae had a marked different carbohydrate composition when compared with the DD-M. vaccae derivatives. As expected, the amount of galactose in the DD-M. vaccae-acid and DD-M. vaccae-periodate derivatives was lower than in DD-M. vaccae and DD-M. vaccae-KOH. These values reflect the action of the acid and periodate in the preparation of the derivatives, cleaving the arabinogalactan sidechains from the peptidoglycan backbone.

Nucleic Acid Analysis of DD-M. vaccae and DD-M. vaccae Derivatives

Analysis by gel electrophoresis of the nucleic acid content of DD-M. vaccae and the DD-M. vaccae derivatives after treatment with Proteinase K showed that DD-M. vaccae, DD-M. vaccae-periodate and DD-M. vaccae-KOH contained small amounts of DNA while no detectable nucleic acid was observed for DD-M. vaccae-acid.

### EXAMPLE 3

### Effect of Immunization with DD-M. vaccae and Derivatives of DD-M. vaccae on Asthma in Mice

The ability of DD-M. vaccae and derivatives of DD-M. vaccae to inhibit the development of allergic immune responses was examined in a mouse model of the asthma-like allergen specific lung disease. The severity of this allergic disease is reflected in the large numbers of eosinophils that accumulate in the airways.

BALB/cByJ mice were given 2 μg ovalbumin in 2 mg alum adjuvant by the intraperitoneal route at time 0 and 14 days, and subsequently given 100 μg ovalbumin in 50 μl phosphate buffered saline (PBS) by the intranasal route on day 28. The mice accumulated eosinophils in their airways as detected by washing the airways of the anesthetized mice with saline, collecting the washings (broncheolar lavage or BAL), and counting the numbers of eosinophils.

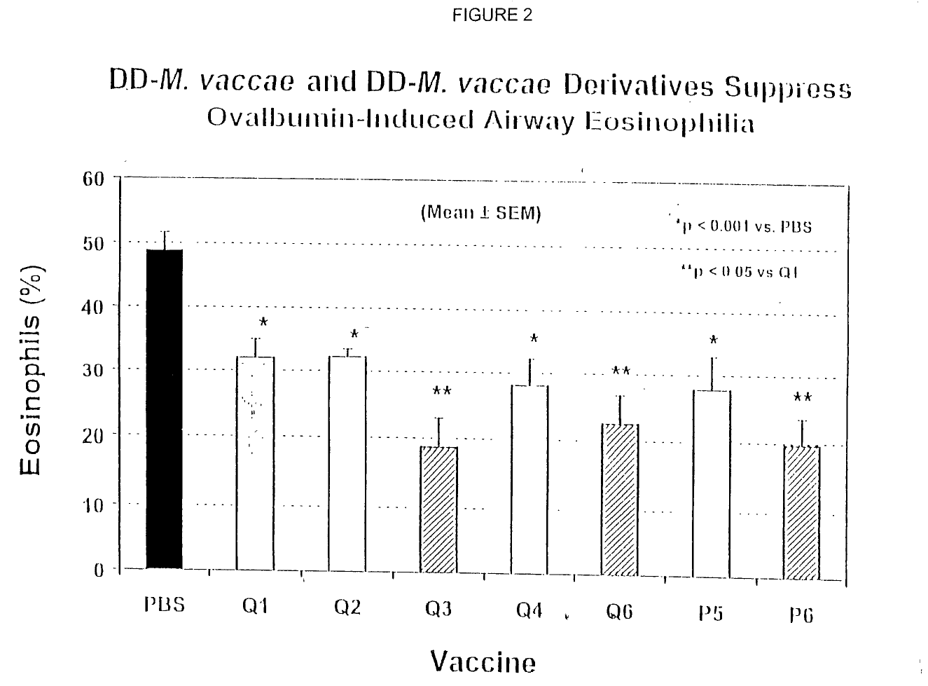

DD-M. vaccae derivatives were prepared as described above. Groups of 10 mice were administered 200 μg of PBS, DD-M. vaccae or one of the DD-M. vaccae derivatives (Q1: DD-M. vaccae; Q2: DD-M. vaccae-KOH; Q3: DD-M. vaccae-acid; Q4: M. vaccae-periodate; Q6 and P6: DD-M. vaccae-KOH-periodate; P5: DD-M. vaccae-KOH-acid) intranasally one week before intranasal challenge with ovalbumin. As shown in FIG. 2, statistically significant reductions were observed in the percentage of eosinophils in BAL cells collected six days after challenge with ovalbumin, compared to control mice. Furthermore, the data shows that suppression of airway eosinophilia with DD-M. vaccae-acid and DD-M. vaccae-KOH-periodate (Q3, Q6 and P6) was greater than that obtained with DD-M. vaccae (Q1). Control mice were given intranasal PBS. The data in FIG. 2 shows the mean and SEM per group of mice.

Eosinophils are blood cells that are prominent in the airways in allergic asthma. The secreted products of eosinophils contribute to the swelling and inflammation of the mucosal linings of the airways in allergic asthma. The data shown in FIG. 2 indicate that treatment with DD-M. vaccae or derivatives of DD-M. vaccae reduces the accumulation of lung eosinophils, and may be useful in reducing inflammation associated with eosinophilia in the airways, nasal mucosal and upper respiratory tract. Administration of DD-M. vaccae or derivatives of DD-M. vaccae may therefore reduce the severity of asthma and diseases that involve similar immune abnormalities, such as allergic rhinitis, atopic dermatitis and eczema.

In addition, serum samples were collected from mice immunized with either heat-killed M. vaccae or DD-M. vaccae and the level of antibodies to ovalbumin was measured by standard enzyme-linked immunoassay (EIA). As shown in Table 3 below, sera from mice infected with BCG had higher levels of ovalbumin-specific IgG1 than sera from PBS controls. In contrast, mice immunized with heat-killed M. vaccae or DD-M. vaccae had similar or lower levels of ovalbumin-specific IgG1. As IgG1 antibodies are characteristic of a Th2 immune response, these results are consistent with the suppressive effects of DD-M. vaccae on the asthma-inducing Th2 immune responses.

TABLE 3
Low Antigen-Specific IgG1 Serum Levels in Mice Immunized
with Heat-killed M. vaccae or DD-M. vaccae

Serum IgG1

| Treatment Group | Mean | SEM |
|---|---|---|
| M. vaccae i.n. | 185.00 | 8.3 |
| M. vaccae s.c. | 113.64 | 8.0 |
| DD-M. vaccae i.n. | 96.00 | 8.1 |
| DD-M. vaccae s.c. | 110.00 | 4.1 |
| BCG, Pasteur | 337.00 | 27.2 |
| BCG, Connaught | 248.00 | 46.1 |
| PBS | 177.14 | 11.4 |

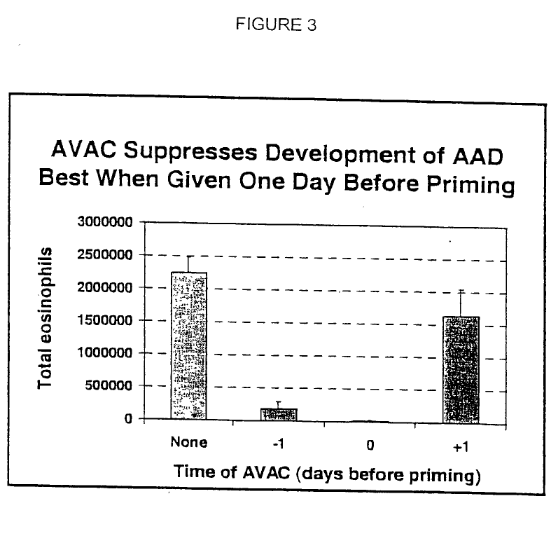

In further studies, the effects of DD-M. vaccae-acid (AVAC) on eosinophilia in the mouse model when administered either one day before challenge with OVA, at the time of challenge or one day after challenge were examined. As shown in FIG. 3, suppression of eosinophilia was greatest when AVAC was administered one day before challenge or at the same time.

### EXAMPLE 4

### Effect of DD-M. vaccae Derivatives on IL-10 Production in THP-1 Cells

IL-10 has been shown to inhibit the cytokine production of Th1 cells and play a key role in the suppression of experimentally-induced inflammatory responses in skin (Berg et al., J. Exp. Med. 182:99-108, 1995). More recently, IL-10 has been used successfully in two clinical trials to treat psoriatic patients (Reich et al., J. Invest. Dermatol. 111:1235-1236, 1998 and Asadullah et al., J. Clin. Invest. 101:783-794, 1998). The levels of IL-10 produced by a human monocytic cell line (THP-1) cultured in the presence of derivatives of DD-M. vaccae were assessed as follows.

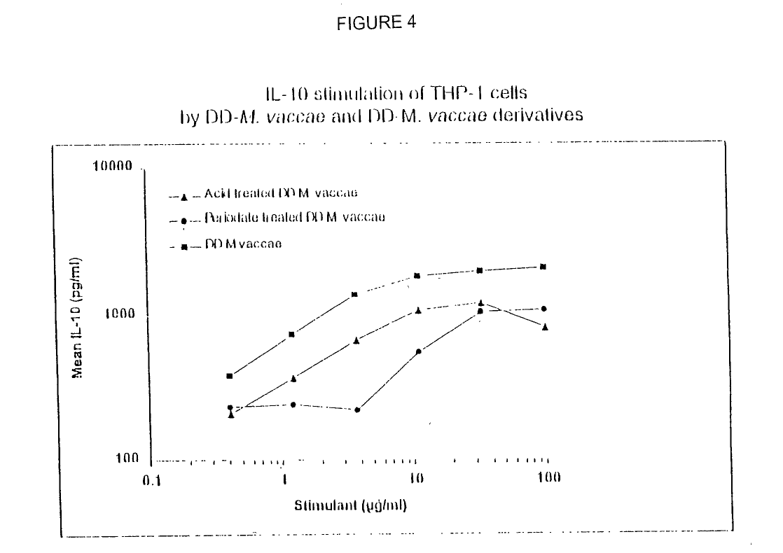

THP-1 cells (ATCC Number TIB-202) were cultured in RPMI medium (Gibco BRL Life Technologies) supplemented with 0.5 mg/l streptomycin, 500 U/1 penicillin, 2 mg/l L-glutamine, 5×10⁻⁵ M β-mercaptoethanol and 5% fetal bovine serum (FBS). One day prior to the assay, the cells were subcultured in fresh media at 5×10⁵ cells/ml. Cells were incubated at 37° C. in humidified air containing 5% CO₂ for 24 hours and then aspirated and washed by centrifugation with 50 ml of media. The cells were resuspended in 5 ml of media and the cell concentration and viability determined by staining with Trypan blue (Sigma, St Louis Mo.) and analysis under a hemocytometer. DD-M. vaccae derivatives (prepared as described above) in 50 μl PBS and control stimulants were added in triplicate to wells of a 96 well plate containing 100 μl of medium and appropriate dilutions were prepared. Lipopolysaccharide (LPS) (300μg/ml; Sigma) and PBS were used as controls. To each well, 100 μl of cells were added at a concentration of 2×10⁶ cells/ml and the plates incubated at 37° C. in humidified air containing 5% CO₂ for 24 hours. The level of IL-10 in each well was determined using human IL-10 ELISA reagents (PharMingen, San Diego Calif.) according to the manufacturer's protocol. As shown in FIG. 4, the acid and periodate derivatives of DD-M. vaccae were found to stimulate significant levels of IL-10 production. The PBS control, DD-M. vaccae-KOH, DD-M. vaccae-KOH-periodate, and DD-M. vaccae-KOH-acid derivatives did not stimulate THP-1 cells to produce IL-10.

### EXAMPLE 5

### Preparation and Compositional Analysis of Delipidated and Deglycolipidated M. tuberculosis (DD-M. tuberculosis) and M. smegmatis (DD-M. smegmatis)

M. tuberculosis and M. smegmatis Culture Filtrate

Cultures of Mycobacterium smegmatis (M. smegmatis, ATCC Number 27199) were grown as described in Example 1 for M. vaccae in Medium 90 with 1% added glucose. After incubation at 37° C. for 5 days, the cells were harvested by centrifugation and the culture filtrate removed. The bacterial pellet was resuspended in phosphate buffered saline at a concentration of 10 mg/ml, equivalent to 10¹⁰ M. smegmatis organisms per ml. The cell suspension was then autoclaved for 15 min at 120° C. The culture filtrate was passaged through a 0.45 μm filter into sterile bottles.

Cultures of M. tuberculosis strain H37Rv (ATCC Number 27294) were grown at 37° C. in GAS medium (0.3 g Bactocasitone (Difco Laboratories, Detroit Mich.), 0.05 g ferric ammonium citrate, 4 g K₂HPO₄, 2 g citric acid, 1 g L-alanine, 1.2 g MgCl₂.6H₂O, 0.6 g K₂ SO₄, 2 g NH₄Cl, 1.8 ml NaOH (10 N), 5 ml glycerol, pH 7.0) for five days. Harvesting and further treatment of cells are as described above for M. smegmatis cells.

Preparation of Delipidated and Deglycolipidated M. tuberculosis (DD-M. tuberculosis) and Delipidated and Deglycolipidated M. smegmatis (DD-M. smegmatis) and Compositional Analysis.

To prepare delipidated and deglycolipidated M. tuberculosis (DD-M. tuberculosis) and M. smegmatis (DD-M. smegmatis), autoclaved M. tuberculosis and M. smegmatis were pelleted by centrifugation, the pellet washed with water and collected again by centrifugation, and freeze-dried. An aliquot of this freeze-dried M. tuberculosis and M. smegmatis was set aside and referred to as lyophilized M. tuberculosis and M. smegmatis, respectively. When used in experiments, the lyophilized material was resuspended in PBS to the desired concentration.

Delipidated and deglycolipidated M. tuberculosis (DD-M. tuberculosis) and M. smegmatis (DD-M. smegmatis) were prepared as described in Example 1 for the preparation of DD-M. vaccae. For bioassay, the freeze-dried DD-M. tuberculosis and DD-M. smegmatis were resuspended in phosphate-buffered saline (PBS) by sonication, and sterilized by autoclaving.

The compositional analyses of DD-M. tuberculosis and DD-M. smegmatis are presented in Table 4 and Table 5. Major differences are seen in some components of the monosaccharide composition of DD-M. tuberculosis and DD-M. smegmatis compared with the monosaccharide composition of DD-M. vaccae. The data presented in Table 4 show that DD-M. tuberculosis and DD-M. smegmatis contain 1.3% and 0.0 mol % glucose, respectively, compared with 28.1 mol % for DD-M. vaccae.

The amino acid composition of DD-M. tuberculosis and DD-M. smegmatis is presented in Table 5. DD-M. tuberculosis contains 6537.9 nmoles/mg amino acids, or approximately 78.5% w/w, and DD-M. smegmatis contains 6007.7 nmoles/mg amino acids, which is approximately 72.1% w/w protein. When compared with the amino acid analysis of DD-M. vaccae, DD-M. tuberculosis and DD-M. smegmatis contain more total % protein than DD-M. vaccae (55.1%).

TABLE 4
Monosaccharide Composition of DD-M. tuberculosis
and DD-M. smegmatis

| | |
|---|---|
| M. tuberculosis | M. smegmatis |

| Monosaccharide | wt % | mol % | wt % | mol % |
|---|---|---|---|---|
| Inositol | 0.0 | 0.0 | 0.0 | 0.0 |
| Glycerol | 9.5 | 9.7 | 15.2 | 15.5 |
| Arabinose | 69.3 | 71.4 | 69.3 | 70.0 |
| Xylose | ND* | ND | 3.9 | 4.0 |
| Mannose | 3.5 | 3.0 | 2.2 | 1.9 |
| Glucose | 1.5 | 1.3 | 0.0 | 0.0 |
| Galactose | 12.4 | 10.7 | 9.4 | 8.0 |

*Not done

TABLE 5
Amino Acid Composition of DD-M. tuberculosis
and DD-M. smegmatis

| | M. tuberculosis | | M. smegmatis | |
|---|---|---|---|---|
| | Total Protein | Total % | Total Protein | Total % |
| Amino acid | nmoles/mg | protein | nmoles/mg | protein |
| ASP | 592.5 | 9.1 | 557.0 | 9.3 |
| THR | 348.1 | 5.3 | 300.5 | 5.0 |
| SER | 218.6 | 3.3 | 252.6 | 4.2 |
| GLU | 815.7 | 12.5 | 664.9 | 11.1 |
| PRO | 342.0 | 5.2 | 451.9 | 7.5 |
| GLY | 642.9 | 9.8 | 564.7 | 9.4 |
| ALA | 927.9 | 14.2 | 875.1 | 14.6 |
| CYS | 31.8 | 0.5 | 20.9 | 0.3 |
| VAL | 509.7 | 7.8 | 434.8 | 7.2 |
| MET | 122.6 | 1.9 | 113.1 | 1.9 |
| ILE | 309.9 | 4.7 | 243.5 | 4.1 |
| LEU | 542.5 | 8.3 | 490.8 | 8.2 |
| TYR | 116.0 | 1.8 | 108.3 | 1.8 |
| PHE | 198.9 | 3.0 | 193.3 | 3.2 |
| HIS | 126.1 | 1.9 | 117.2 | 2.0 |
| LYS | 272.1 | 4.2 | 247.8 | 4.1 |
| ARG | 421.0 | 6.4 | 371.7 | 6.2 |

### EXAMPLE 6

### Effect of Immunization with DD-M. tuberculosis and DD-M. smegmatis on Asthma in Mice

The ability of DD-M. tuberculosis and DD-M. smegmatis to inhibit the development of allergic immune responses was examined in a mouse model of the asthma-like allergen-specific lung disease, as described above in Example 3. The results illustrate the effect of immunization with DD-M. tuberculosis and DD-M. smegmatis on the suppression of eosinophilia in the airways, illustrating their immune modulating properties.

BALB/cByJ female mice were sensitized to OVA by intraperitoneal injection of 200 μl of an emulsion containing 10 μg OVA and 1 mg Alum adjuvant on days 0 and 7. On days 14 and 21, mice were anesthetized and vaccinated intranasally or intradermally with 200 μg of DD-M. vaccae, DD-M. tuberculosis, DD-M. smegmatis or PBS. On days 28 and 32, mice were anesthetized and challenged intranasally with 100 μg OVA. Mice were sacrificed on day 35 and bronchoalveolar lavage (BAL) performed using PBS. BAL cell samples were analyzed by flow cytometry to determine the eosinophil content (% eosinophils). Total BAL eosinophil numbers were obtained by multiplying the percentage eosinophil value by the total number of leukocytes obtained, with the latter value being determined using a hemacytometer.

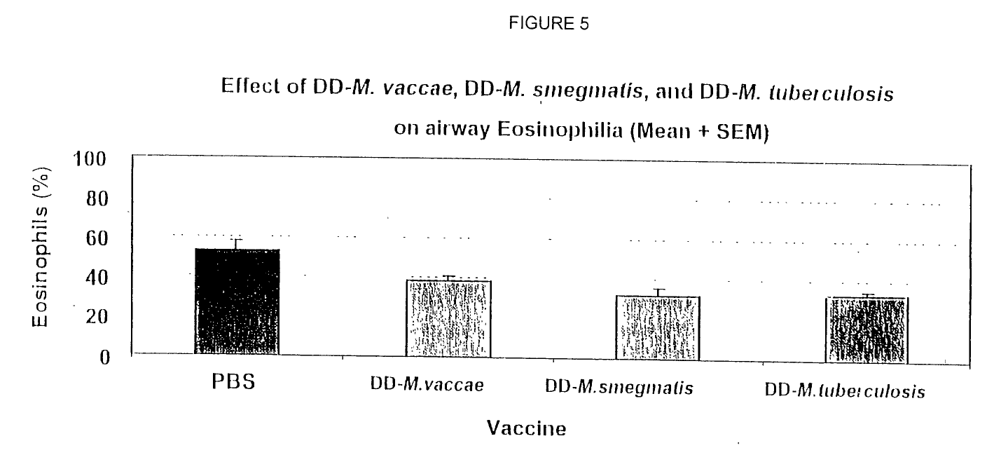

The data shown in FIG. 5 indicate that treatment with DD-M. tuberculosis and DD-M. smegmatis reduces the accumulation of lung eosinophils similar to the reduction following immunization with DD-M. vaccae, and that DD-M. tuberculosis and DD-M. smegmatis may be useful in reducing inflammation associated with eosinophilia in the airways, nasal mucosal and upper respiratory tract. Administration of DD-M. tuberculosis and DD-M. smegmatis may therefore reduce the severity of asthma and diseases that involve similar immune abnormalities, such as allergic rhinitis.

### EXAMPLE 7

### Effect of DD-M. vaccae on Cyctokine Production in Human Peripheral Blood Mononuclear Cells

This example describes studies on the ability of DD-M. vaccae to stimulate production of IL-10, TNF-α and IFN-γ in human peripheral blood mononuclear cells (PBMC).

Human blood was separated into PBMC and non-adherent cells, and the cytokine production of each fraction determined after stimulation with DD-M. vaccae as follows. Blood was diluted with an equal volume of saline and 15-20 ml was layered onto 10 ml Ficoll (Gibco BRL Life Technologies, Gaithersburg, Md.). The lymphocyte layer was removed after centrifugation at 1,800 rpm for 20 min, washed three times in RPMI medium (Gibco BRL) and counted using Trypan blue. Cells were resuspended in RPMI containing 5% heat-inactivated autologous serum at a concentration of 2×10⁶ per ml. The cell sample was divided to prepare non-adherent cells.

Non-adherent cells were prepared by incubating 20 ml of the lymphocytes in RPMI supplemented with serum (as above) for one hour in a humidified atmosphere containing 5% CO₂. The non-adherent cells were transferred to a fresh flask and the incubation repeated once more. The non-adherent cells were removed, counted and resuspended at a concentration of 2×10⁶ per ml in supplemented RPMI medium. Serial dilutions of DD-M. vaccae were prepared starting at 200 μg/ml and added to 100 μl medium (supplemented RPMI) in a 96-well plate. PBMC and non-adherent cells were added to the wells (100 μl) and the plates incubated at 37° C. for 48 hours in a humidified atmosphere containing 5% CO₂. A 150 μl aliquot was removed from each well to determine the amount of cytokine produced by the different cells after stimulation with DD-M. vaccae.

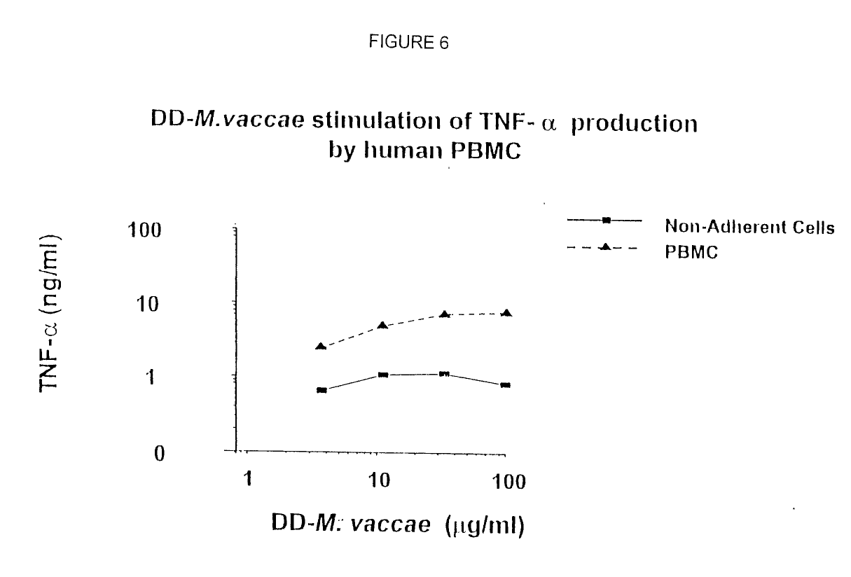

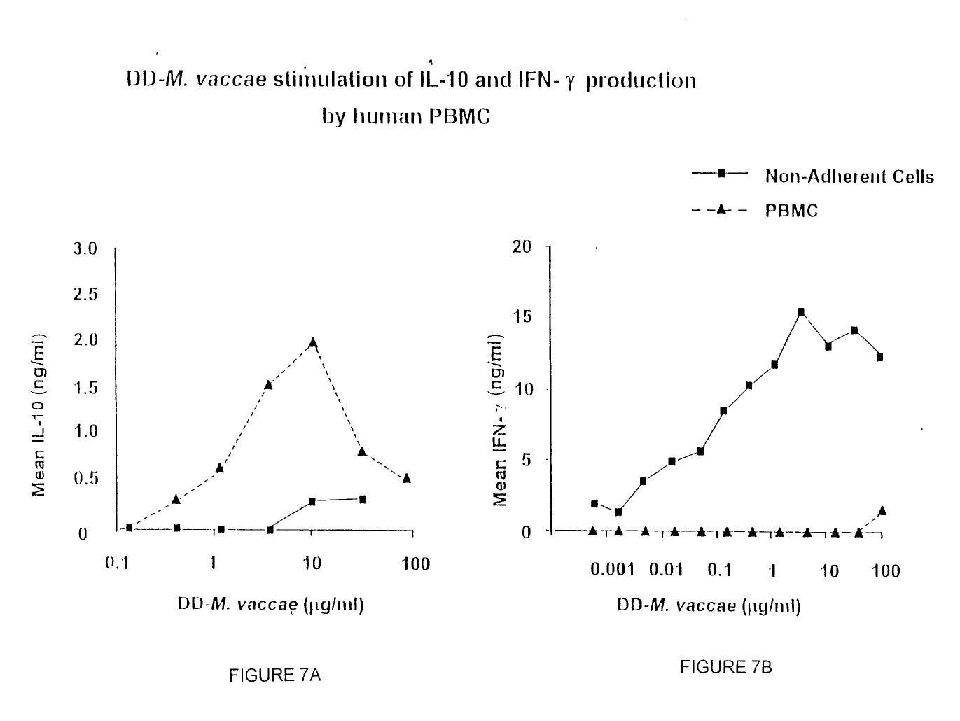

DD-M. vaccae stimulated PBMC to secrete TNF-α and IL-10 (FIGS. 6 and 7A, respectively), but stimulated the non-adherent cells to produce IFN-γ (FIG. 7B). These data suggest that IFN-γ production in DD-M. vaccae-stimulated PBMC is repressed by the simultaneous secretion of IL-10.

### EXAMPLE 8

### Effect of Intradermal Injection of Heat-Killed Mycobacterium vaccae on Psoriasis in Human Patients

This example illustrates the effect of two intradermal injections of heat-killed Mycobacterium vaccae on psoriasis.

M. vaccae (ATCC Number 15483) was cultured in sterile Medium 90 (yeast extract, 2.5 g/l; tryptone, 5 g/l; glucose, 1 g/l) at 37° C. The cells were harvested by centrifugation, and transferred into sterile Middlebrook 7H9 medium (Difco Laboratories, Detroit, Mich., USA) with glucose at 37° C. for one day. The medium was then centrifuged to pellet the bacteria, and the culture filtrate removed. The bacterial pellet was resuspended in phosphate buffered saline at a concentration of 10 mg/ml, equivalent to 10¹⁰ M. vaccae organisms per ml. The cell suspension was then autoclaved for 15 min at 120° C. and stored frozen at −20° C. Prior to use the M. vaccae suspension was thawed, diluted to a concentration of 5 mg/ml in phosphate buffered saline, autoclaved for 15 min at 120° C. and 0.2 ml aliquoted under sterile conditions into vials for use in patients.

Twenty four volunteer psoriatic patients, male and female, 15-61 years old with no other systemic diseases were admitted to treatment. Pregnant patients were not included. The patients had PASI scores of 12-35. The PASI score is a measure of the location, size and degree of skin scaling in psoriatic lesions on the body. A PASI score of above 12 reflects widespread disease lesions on the body. The study commenced with a washout period of four weeks where the patients did not have systemic anti-psoriasis treatment or effective topical therapy.

The 24 patients were then injected intradermally with 0.1 ml M. vaccae (equivalent to 500 μg). This was followed three weeks later with a second intradermal injection with the same dose of M. vaccae (500 μg). Psoriasis was evaluated from four weeks before the first injection of heat-killed M. vaccae to twelve weeks after the first injection as follows:

A. The PASI scores were determined at −4, 0, 3, 6 and 12 weeks;

B. Patient questionnaires were completed at 0, 3, 6 and 12 weeks; and

C. Psoriatic lesions: each patient was photographed at 0, 3, 6, 9 and 12 weeks.

The data shown in Table 6 describe the age, sex and clinical background of each patient.

TABLE 6
Patient Data in the Study of the Effect of M. vaccae in Psoriasis

| Code | | | Duration of | | |
|---|---|---|---|---|---|
| No. | Patient | Age/Sex | Disorder | | Admission PASI Score |
| PS-001 | D. C. | 49/F | 30 | years | 28.8 |
| PS-002 | E. S. | 41/F | 4 | months | 19.2 |
| PS-003 | M. G. | 24/F | 8 | months | 18.5 |
| PS-004 | D. B. | 54/M | 2 | years | 12.2 |
| PS-005 | C. E. | 58/F | 3 | months | 30.5 |
| PS-006 | M. G. | 18/F | 3 | years | 15.0 |
| PS-007 | L. M. | 27/M | 3 | years | 19.0 |
| PS-008 | C. C | 21/F | 1 | month | 12.2 |
| PS-009 | E. G | 42/F | 5 | months | 12.6 |
| PS-010 | J. G | 28/M | 7 | years | 19.4 |
| PS-011 | J. U | 39/M | 1 | year | 15.5 |
| PS-012 | C. S | 47/M | 3 | years | 30.9 |
| PS-013 | H. B | 44/M | 10 | years | 30.4 |
| PS-014 | N. J | 41/M | 17 | years | 26.7 |
| PS-015 | J. T | 61/F | 15 | years | 19.5 |
| PS-016 | L. P | 44/M | 5 | years | 30.2 |
| PS-017 | E. N | 45/M | 5 | years | 19.5 |
| PS-018 | E. L | 28/F | 19 | years | 16.0 |
| PS-019 | B. A | 38/M | 17 | years | 12.3 |
| PS-020 | P. P | 58/F | 1 | year | 13.6 |
| PS-021 | L. I | 27/F | 8 | months | 22.0 |
| PS-022 | A. C | 20/F | 7 | months | 26.5 |
| PS-023 | C. A | 61/F | 10 | years | 12.6 |
| PS-024 | F. T | 39/M | 15 | years | 29.5 |

All patients demonstrated a non-ulcerated, localized erythematous soft indurated reaction at the injection site. No side effects were noted, or complained of by the patients. The data shown in Table 7, below, are the measured skin reactions at the injection site, 48 hours, 72 hours and 7 days after the first and second injections of heat-killed M. vaccae. The data shown in Table 8, below, are the PASI scores of the patients at the time of the first injection of M. vaccae (Day 0) and 3, 6, 9, 12 and 24 weeks later.

It can clearly be seen that, by week 9 after the first injection of M. vaccae, 16 of 24 patients showed a significant improvement in PASI scores. Seven of 14 patients who completed 24 weeks of follow-up remained stable with no clinical sign of redevelopment of severe disease. These results demonstrate the effectiveness of multiple intradermal injections of inactivated M. vaccae in the treatment of psoriasis. PASI scores below 10 reflect widespread healing of lesions. Histopathology of skin biopsies indicated that normal skin structure is being restored. Only one of the first seven patients who completed 28 weeks follow-up had a relapse.

TABLE 7
Skin Reaction Measurements in Millimeter

| | Time of Measurement | | | | | |
|---|---|---|---|---|---|---|
| | First Injection | | | Second Injection | | |
| Code No. | 48 hours | 72 hours | 7 days | 48 hours | 72 hours | 7 days |
| PS-001 | 12 × 10 | 12 × 10 | 10 × 8 | 15 × 14 | 15 × 14 | 10 × 10 |
| PS-002 | 18 × 14 | 20 × 18 | 18 × 14 | 16 × 12 | 18 × 12 | 15 × 10 |
| PS-003 | 10 × 10 | 14 × 10 | 10 × 8 | 15 × 12 | 15 × 10 | 10 × 10 |
| PS-004 | 14 × 12 | 22 × 18 | 20 × 15 | 20 × 20 | 20 × 18 | 14 × 10 |
| PS-005 | 10 × 10 | 13 × 10 | DNR | DNR | DNR | DNR |
| PS-006 | 10 × 8 | 10 × 10 | 6 × 4 | 12 × 10 | 15 × 15 | 10 × 6 |
| PS-007 | 15 × 15 | 18 × 16 | 12 × 10 | 15 × 13 | 15 × 12 | 12 × 10 |
| PS-008 | 18 × 18 | 13 × 12 | 12 × 10 | 18 × 17 | 15 × 10 | 15 × 10 |
| PS-009 | 13 × 13 | 18 × 15 | 12 × 8 | 15 × 13 | 12 × 12 | 12 × 7 |
| PS-010 | 13 × 11 | 15 × 15 | 8 × 8 | 12 × 12 | 12 × 12 | 5 × 5 |
| PS-011 | 17 × 13 | 14 × 12 | 12 × 11 | 12 × 10 | 12 × 10 | 12 × 10 |
| PS-012 | 17 × 12 | 15 × 12 | 9 × 9 | 10 × 10 | 10 × 6 | 8 × 6 |
| PS-013 | 18 × 11 | 15 × 11 | 15 × 10 | 15 × 10 | 15 × 13 | 14 × 6 |
| PS-014 | 15 × 12 | 15 × 11 | 15 × 10 | 13 × 12 | 14 × 10 | 8 × 5 |
| PS-015 | 15 × 12 | 16 × 12 | 15 × 10 | 7 × 6 | 14 × 12 | 6 × 4 |
| PS-016 | 6 × 5 | 6 × 6 | 6 × 5 | 8 × 8 | 9 × 8 | 9 × 6 |
| PS-017 | 20 × 15 | 15 × 14 | 14 × 10 | 15 × 15 | 17 × 16 | DNR |
| PS-018 | 14 × 10 | 10 × 8 | 10 × 8 | 12 × 12 | 10 × 10 | 10 × 10 |
| PS-019 | 10 × 10 | 14 × 12 | 10 × 8 | DNR | 15 × 14 | 15 × 14 |
| PS-020 | 15 × 12 | 15 × 15 | 12 × 15 | 15 × 15 | 14 × 12 | 13 × 12 |
| PS-021 | 15 × 12 | 15 × 12 | 7 × 4 | 11 × 10 | 11 × 10 | 11 × 8 |
| PS-022 | 12 × 10 | 10 × 8 | 10 × 8 | 15 × 12 | 13 × 10 | 10 × 8 |
| PS-023 | 13 × 12 | 14 × 12 | 10 × 10 | 17 × 17 | 15 × 15 | DNR |
| PS-024 | 10 × 10 | 10 × 10 | 10 × 8 | 10 × 8 | 8 × 7 | 8 × 7 |

DNR = Did not report.

TABLE 8
Clinical Status of Patients after Injection of M. vaccae (PASI Scores)

| Code No. | Day 0 | Week 3 | Week 6 | Week 9 | Week 12 | Week 24 |
|---|---|---|---|---|---|---|
| PS-001 | 28.8 | 14.5 | 10.7 | 2.2 | 0.7 | 0 |
| PS-002 | 19.2 | 14.6 | 13.6 | 10.9 | 6.2 | 0.6 |
| PS-003 | 18.5 | 17.2 | 10.5 | 2.7 | 1.6 | 0 |
| PS-004 | 12.2 | 13.4 | 12.7 | 7.0 | 1.8 | 0.2 |
| PS-005* | 30.5 | DNR | 18.7 | DNR | DNR | 0 |
| PS-006 | 15.0 | 16.8 | 16.4 | 2.7 | 2.1 | 3.0 |
| PS-007 | 19.0 | 15.7 | 11.6 | 5.6 | 2.2 | 0 |
| PS-008 | 12.2 | 11.6 | 11.2 | 11.2 | 5.6 | 0 |
| PS-009 | 12.6 | 13.4 | 13.9 | 14.4 | 15.3 | 13.0 |
| PS-010 | 18.2 | 16.0 | 19.4 | 17.2 | 16.9 | 19.3 |
| PS-011 | 17.2 | 16.9 | 16.7 | 16.5 | 16.5 | 15.5 |
| PS-012 | 30.9 | 36.4 | 29.7 | 39.8** | | |
| PS-013 | 19.5 | 19.2 | 18.9 | 17.8 | 14.7 | 17.8 |
| PS-014 | 26.7 | 14.7 | 7.4 | 5.8 | 9.9 | 24.4*** |
| PS-015 | 30.4 | 29.5 | 28.6 | 28.5 | 28.2 | 24.3 |
| PS-016 | 30.2 | 16.8 | 5.7 | 3.2 | 0.8 | |
| PS-017 | 12.3 | 12.6 | 12.6 | 12.6 | 8.2 | |
| PS-018 | 16.0 | 13.6 | 13.4 | 13.4 | 13.2 | |
| PS-019 | 19.5 | 11.6 | 7.0 | DNR | DNR | |
| PS-020 | 13.6 | 13.5 | 12.4 | 12.7 | 12.4 | |
| PS-021 | 22.0 | 20.2 | 11.8 | 11.4 | 15.5 | |
| PS-022 | 26.5 | 25.8 | 20.7 | 11.1 | 8.3 | |
| PS-023 | 12.6 | 9.2 | 6.6 | 5.0 | 4.8 | |
| PS-024 | 29.5 | 27.5 | 20.9 | 19.0 | 29.8 | |

*Patient PS-005 received only one dose of autoclaved M. vaccae.
**Patient PS-012 removed from trial, drug (penicillin) induced dermatitis
***Patient PS-014 was revaccinated
DNR = Did not report
Blank cells indicate pending follow-up

### EXAMPLE 9

### Effect of Intradermal Injection of Delipidated and Deglycolipidated Mycobacterium vaccae (DD-M. vaccae) on Psoriasis in Human Patients

This example illustrates the effect of two intradermal injections of DD-M. vaccae on psoriasis and the lack of T cell proliferation induced in these patients after treatment with DDMV.

Seventeen volunteer psoriatic patients, male and female, 18-48 years old with no other systemic diseases were admitted to treatment. Pregnant patients were not included. The patients had PASI scores of 12-30. As discussed above, the PASI score is a measure of the location, size and degree of skin scaling in psoriatic lesions on the body with a PASI score of above 12 reflecting widespread disease lesions on the body. The study commenced with a washout period of four weeks where the patients did not have systemic anti-psoriasis treatment or effective topical therapy. The 17 patients were then injected intradermally with 0.1 ml DD-M. vaccae (equivalent to 100 μg). This was followed three weeks later with a second intradermal injection with the same dose of DD-M. vaccae (100 μg).

Psoriasis was evaluated from four weeks before the first injection of M. vaccae to 48 weeks after the first injection as follows:

A. the PASI scores were determined at −4, 0, 3, 6, 12, 24, 36 and 48 weeks;

B. patient questionnaires were completed at 0, 3, 6, 9 and 12 weeks, and thereafter every 4 weeks; and

C. psoriatic lesions: each patient was photographed at 0 and 3 weeks, and thereafter at various intervals.

The data shown in Table 9 describe the age, sex and clinical background of each patient.

TABLE 9
Patient Data in the Study of the Effect
of DD-M. vaccae in Psoriasis

| Code | | | Duration of | | |
|---|---|---|---|---|---|
| No. | Patient | Age/Sex | Disorder | | Admission PASI Score |
| PS-025 | A. S | 25/F | 2 | years | 12.2 |
| PS-026 | M. B | 45/F | 3 | months | 14.4 |
| PS-027 | A. G | 34/M | 14 | years | 24.8 |
| PS-028 | E. M | 31/M | 4 | years | 18.2 |
| PS-029 | A. L | 44/M | 5 | months | 18.6 |
| PS-030 | V. B | 42/M | 5 | years | 21.3 |
| PS-031 | R. A | 18/M | 3 | months | 13.0 |
| PS-032 | | 42/M | 23 | years | 30.0 |
| PS-033 | | 37/F | 27 | years | 15.0 |
| PS-034 | | 42/M | 15 | years | 30.4 |
| PS-035 | | 35/M | 6 | years | 13.2 |
| PS-036 | | 43/M | 6 | years | 19.5 |
| PS-037 | | 35/F | 4 | years | 12.8 |
| PS-038 | | 44/F | 7 | months | 12.6 |
| PS-039 | | 20/F | 1 | year | 16.1 |
| PS-040 | | 28/F | 8 | months | 25.2 |
| PS-041 | | 48/F | 10 | years | 20.0 |

All patients demonstrated a non-ulcerated, localized erythematous soft indurated reaction at the injection site. No side effects were noted, or complained of by the patients. The data shown in Table 10 are the measured skin reactions at the injection site, 48 hours, 72 hours and 7 days after the first injection of DD-M. vaccae, and 48 hours and 72 hours after the second injection.

TABLE 10
Skin Reaction Measurements in Millimeters

| | Time of Measurement | | | | |
|---|---|---|---|---|---|
| | First Injection | | | Second Injection | |
| Code No. | 48 hours | 72 hours | 7 days | 48 hours | 72 hours |
| PS-025 | 8 × 8 | 8 × 8 | 3 × 2 | 10 × 10 | 10 × 10 |
| PS-026 | 12 × 12 | 12 × 12 | 8 × 8 | DNR | 14 × 14 |
| PS-027 | 9 × 8 | 10 × 10 | 10 × 8 | 9 × 5 | 9 × 8 |
| PS-028 | 10 × 10 | 10 × 10 | 10 × 8 | 10 × 10 | 10 × 10 |
| PS-029 | 8 × 6 | 8 × 6 | 5 × 5 | 8 × 8 | 8 × 8 |
| PS-030 | 14 × 12 | 14 × 14 | 10 × 10 | 12 × 10 | 12 × 10 |
| PS-031 | 10 × 10 | 12 × 12 | 10 × 6 | 14 × 12 | 12 × 10 |

DNR = Did not report

The data shown in Table 11 are the PASI scores of the 17 patients at the time of the first injection of DD-M. vaccae (Day 0), then 3, 6, 12, 24, 36 and 48 weeks later, when available.

TABLE 11
Clinical Status of Patients after Injection of DD-M. vaccae (PASI Scores)

| Code | | | | | | | | Repeat |
|---|---|---|---|---|---|---|---|---|
| No. | Day 0 | Week 3 | Week 6 | Week 12 | Week 24 | Week 36 | Week 48 | treatment |
| PS-025 | 12.2 | 4.1 | 1.8 | 1.4 | 1.7 | 0.2 | 15.8 | Wk 48 |
| PS-026 | 14.4 | 11.8 | 6.0 | 6.9 | 1.4 | 0.4 | | |
| PS-027 | 24.8 | 23.3 | 18.3 | 9.1 | 10.6 | 7.5 | 1.9 | |
| PS-028 | 18.2 | 24.1 | 28.6* | | | | | |
| PS-029 | 18.6 | 9.9 | 7.4 | 3.6 | 0.8 | 0 | 0 | |
| PS-030 | 21.3 | 15.7 | 13.9 | 16.5 | 18.6 | 5.8 | 1.7 | |
| PS-031 | 13.0 | 5.1 | 2.1 | 1.6 | 0.3 | 0 | 0 | |
| PS-032 | 30.0 | 28.0 | 20 | 12.4 | 20.4 | 19.0 | 21.5 | Wk 44 |
| PS-033 | 19.0 | 12.6 | 5.9 | 4.0 | 12.6 | 21.1 (wk 40) | 7.1 (wk 52) | Wk 20 |
| PS-034 | 30.4 | 31.2 | 31.6 | 32.4 | 25.5 | 33.0 | | Wk 20 |
| PS-035 | 13.2 | 11.6 | 10.6 | 1.6 | 1.4 (wk 20) | 1.0 | | |
| PS-036 | 19.5 | 18.0 | 18.0 | 16.8 | 18.0 | 10.2 | | Wk 20, 32 |
| PS-037 | 12.8 | 13.1 | 1.2 | 0 | 0 | 0 | | |
| PS-038 | 12.6 | 12.6 | 12.7 | 10.0 | | | | Wk 12 |
| PS-039 | 16.1 | 17.9 | 18.3 | 17.0 | | | | Wk 12 |
| PS-040 | 25.2 | 3.9 | 0.5 | | | | | |
| PS-041 | 20.0 | 12.7 | 0.8 | | | | | |

*Patient PS-28 removed from trial, exfoliative dermatitis/psoriasis
Blank cells indicate pending follow-up
Wk—weeks after first injection

These results show the significant improvement in PASI scores in 16 patients after injection with DD-M. vaccae. One patient dropped out of the study at 12 weeks with the diagnosis of exfoliative dermatitis/psoriasis. Patients who relapsed received a second or third injection of DD-M. vaccae at the time indicated in Table 11.

At 6 weeks follow-up (n=17), the PASI score improved by >50% in 9 of 17 (53%) patients. At 12 weeks follow up (n=14), the PASI score improved by >50% in 9 of 14 (64.3%) patients. Seven of these patients showed significant clinical improvement with reduction in PASI score to less than 8. At 24 weeks follow up (n=12), the PASI score improved by >50% in 7 of 12 (58%) patients and at 48 weeks follow up (n=7), the PASI score improved by >50% in 5 of 7 (71%) patients. Again, four of these patients showed significant clinical improvement with reduction in PASI score to less than 2. Local injections of DD-M. vaccae were observed to result in clearance of skin lesions distant from the site of injection.

Lack of DDMV-specific T-cell Proliferative Response in Peripheral Blood Cells from Patients Treated with DDMV

In a lymphocyte proliferation assay, the proliferative effect of DDMV on PBMC from the psoriasis patients after treatment with DDMV was determined. A few of these patients were known to be PPD (purified protein derivative from M. bovis) skin test positive and their T cells were shown to proliferate in response to PPD. Donor PBMCs were cultured in medium comprising RPMI 1640 supplemented with 10% (v/v) autologous serum, penicillin (60 mg/ml), streptomycin (100 mg/ml), and glutamine (2 mM) with DDMV (12.5 and 6.25 μg), or heat killed M.vaccae (6.25, 12.5, 25 or 50 μg/ml) or PPD (10 or 1 μg).

The plates were cultured for 7 days and then pulsed with lmCi/well of tritiated thymidine for a further 18 hours, harvested and tritium uptake determined using a scintillation counter. Fractions that stimulated proliferation in both replicates two-fold greater than the proliferation observed in cells cultured in medium alone were considered positive.

The data in Table 12 shows that treatment with DDMV at 0 weeks did not enhance T cell proliferative response to DDMV nor M. vaccae 6 to 15 weeks later. Generally, treatment with DDMV also did not enhance T cell responses to PPD. Cells from all donors did proliferate in vitro upon stimulation with a positive mitogen control, phytohemagglutinnin (PHA).

TABLE 12
Induction of T-cell proliferation in peripheral
blood cells from patients treated with DDMV.

| | Time | PPD | | M. vaccae | | | | DDMV | | |
|---|---|---|---|---|---|---|---|---|---|---|
| Patient | after | 10 | 1 | | 25 | 12.5 | 6.25 | | 6.25 | PHA |
| No | injection | μg | μg | 50 μg | μg | μg | μg | 12.5 μg | μg | 10 |
| 025 | D0 | 2.6* | 1.2 | 1.2 | 0.95 | 1.4 | 1.1 | nd | nd | 21 |
| | 6 wks | 2.8 | 2.9 | 1.4 | 2.0 | 1.7 | 1.5 | nd | nd | 19.8 |
| | 13 wks | 1.4 | 1.0 | 1.5 | 1.3 | 1.3 | 2.3 | 2.6 | 1.3 | 28.4 |
| 026 | D0 | 3.4 | 2.1 | 1.3 | 1.1 | 1.5 | 1.1 | nd | nd | 11.4 |
| | 6 wks | 1.7 | 1.4 | 0.98 | 1.2 | 1.2 | 1.3 | nd | nd | 12 |
| | 13 wks | 2.0 | 1.1 | 0.8 | 1.1 | 1.5 | 1.5 | 1.3 | 1.0 | 29 |
| 027 | D0 | 1.2 | 0.99 | 0.73 | 1.0 | 1.1 | 1.1 | nd | nd | 12.4 |
| | 6 wks | 0.8 | 0.8 | 0.61 | 0.59 | 0.77 | 0.74 | nd | nd | 6.9 |
| | 13 wks | 0.82 | 1.0 | 1.0 | 0.8 | 1.0 | 0.9 | 0.78 | 1.1 | 16.9 |
| 028 | D0 | 1.9 | 1.4 | 1.0 | 1.1 | 1.1 | 1.1 | nd | nd | 24.4 |
| | 6 wks | 1.4 | 1.0 | 0.95 | 0.97 | 0.8 | 0.8 | nd | nd | 14.7 |
| | 14 wks | 2.0 | 0.9 | 0.8 | 1.0 | 1.2 | 1.3 | 0.8 | 0.9 | 156 |
| 029 | D0 | 1.2 | 1.1 | 1.7 | 1.5 | 1.7 | 1.7 | nd | nd | 20 |
| | 5 wks | nd | nd | nd | nd | nd | nd | nd | nd | ND |
| | 12 wks | 3.5 | 1.1 | 1.2 | 1.2 | 1.3 | 1.1 | 1.0 | 1.1 | 154 |
| 030 | D0 | 2.0 | 1.2 | 1.4 | 1.6 | 1.2 | 1.2 | nd | nd | 21 |
| | 5 wks | nd | nd | nd | nd | nd | nd | nd | nd | nd |
| | 12 wks | 4.0 | 2.4 | 1.8 | 2.1 | 0.9 | 1.0 | 2.1 | 1.5 | 380 |
| 031 | D0 | 1.7 | 1.3 | 0.88 | 1.0 | 0.81 | 0.92 | nd | nd | 15 |
| | 5 wks | nd | nd | nd | nd | nd | nd | nd | nd | nd |
| | 12 wks | 9.3 | 5.3 | 1.4 | 1.1 | 1.3 | 0.7 | 1.5 | 1.6 | 329 |
| 032 | D0 | 4.8 | 2.3 | 1.4 | 1.3 | 0.94 | 1.4 | 1.8 | 1.3 | 98 |
| | 6 wks | 5.7 | 1.9 | 1.9 | 1.5 | 1.4 | 1.0 | 1.4 | 1.3 | 32 |
| | 15 wks | 2.4 | 3.3 | 0.6 | 0.54 | 0.7 | 0.9 | 1.4 | 0.9 | 74 |
| 033 | D0 | 0.7 | 1.0 | 1.4 | 0.74 | 1.7 | 1.5 | 1.7 | 1.4 | 709 |
| | 6 wks | 1.3 | 1.5 | 1.2 | 1.1 | 0.8 | 1.3 | 1.1 | 1.1 | 168 |
| | 12 wks | 0.85 | 1.1 | 1.3 | 1.2 | 0.96 | 1.4 | 1.7 | 2.1 | 211 |
| 034 | D0 | 3.1 | 1.2 | 1.4 | 1.1 | 1.0 | 1.3 | 1.1 | 1.0 | 110 |
| | 6 wks | 4.0 | 1.3 | 0.9 | 0.8 | 0.7 | 0.7 | 1.7 | 1.4 | 213 |
| | 12 wks | 3.0 | 0.6 | 1.4 | 0.9 | 0.5 | 0.5 | 1.0 | 0.9 | 72 |
| 035 | D0 | 4.0 | 1.7 | 2.5 | 1.3 | 1.4 | 1.4 | 2.8 | 1.4 | 232 |
| | 6 wks | 3.2 | 1.5 | 2.8 | 1.4 | 1.6 | 1.4 | 1.8 | 2.6 | 670 |
| | 12 wks | 1.2 | 0.5 | 0.8 | 1.1 | 1.2 | 0.4 | 0.9 | 0.6 | 38 |
| 036 | D0 | 2.3 | 1.5 | 1.1 | 0.7 | 1.0 | 0.9 | 2.1 | 1.1 | 182 |
| | 6 wks | 5.7 | 4.2 | 1.6 | 1.5 | 1.9 | 2.6 | 2.4 | 1.4 | 243 |
| | 12 wks | 5.9 | 2.1 | 2.7 | 1.9 | 1.7 | 1.5 | 2.9 | 1.56 | 153 |
| 037 | D0 | 3.3 | 3.2 | 1.8 | 1.5 | 1.2 | 1.8 | 1.9 | 1.5 | 145 |
| | 6 wks | 6.8 | 3.3 | 1.1 | 0.8 | 0.5 | 0.5 | 1.1 | 0.8 | 82 |
| | 12 wks | 10.3 | 3.6 | 2.9 | 1.6 | 1.4 | 1.4 | 1.5 | 2.0 | 55 |

Nd—not done
Values expressed as Stimulation Index (SI) = cpm from tritiated thymidine uptake in presence of DDMV/cpm in absence of DDMV
D0—Blood sample taken prior to first treatment
Wks—weeks

### EXAMPLE 10

### Immunogenicity and Immunomodulating Properties of Recombinant Proteins Derived from M. vaccae and DD-M. vaccae

A. Induction of T Cell Proliferation and IFN-γ Production

The polynucleotide sequences for the M. vaccae antigens GV-1/70, GV-1/83, GV-3, GV4P, GV-5, GV-5P, GV-7, GV-9, GV-13, GV-14, GV-22B, GV-23, GV-24B, GV-27, GV-27A, GV-27B, GV-29, GV-33, GV-35, GV-38AP, GV-38BP, GV-40P, GV-41B, GV-42, GV-44 and GV-45 are provided in SEQ ID NO: 1-26, respectively, with the corresponding amino acid sequences being provided in SEQ ID NO: 27-52, respectively. The isolation of these antigens and additional information and characterization of these antigens is described in U.S. Pat. No. 6,160,093, the disclosure of which is hereby incorporated herein by reference in its entirety.

The immunogenicity of Mycobacterium vaccae recombinant proteins (referred to herein as GV recombinant proteins) was tested by injecting female BALB/cByJ mice in each hind foot-pad with 10 μg of recombinant GV proteins emulsified in incomplete Freund's adjuvant (IFA). Control mice received phosphate buffered saline in IFA. The draining popliteal lymph nodes were excised 10 days later and the cells obtained therefrom were stimulated with the immunizing GV protein and assayed for proliferation by measuring the uptake of tritiated thymidine. The amount of interferon gamma (IFNγ) produced and secreted by these cells into the culture supernatants was assayed by standard enzyme-linked immunoassay.

As shown in Table 13, all GV proteins were found to induce a T cell proliferative response. The lymph node T cells from immunized mice proliferated in response to the specific GV protein used in the immunization. Lymph node cells from non-immunized mice did not proliferate in response to GV proteins. The data in Table 14 showing IFNγ production, indicate that most of the GV proteins stimulated IFNγ production by lymph node cells from mice immunized with the corresponding GV protein. When lymph node cells from non-immunized mice were cultured with individual GV proteins, IFNγ production was not detectable. The GV proteins are thus able to stimulate T cell proliferation and/or IFNγ production when administered by subcutaneous injection.

TABLE 13
Immunogenic Properties of GV proteins: Proliferation

| | Proliferation (cpm) | | |
|---|---|---|---|
| | Dose of GV protein used in vitro (μg/ml) | | |
| GV protein | 50 | 2 | 0.08 |
| GV-1/70 | 31,550 ± 803 | 19,058 ± 2,449 | 5,596 ± 686 |
| GV-1/83 | 18,549 ± 2,716 | 23,932 ± 1,964 | 11,787 ± 1,128 |
| GV-3 | 34,751 ± 1,382 | 6,379 ± 319 | 4,590 ± 1,042 |
| GV-4P | 26,460 ± 1,877 | 10,370 ± 667 | 6,685 ± 673 |
| GV-5 | 42,418 ± 2,444 | 23,902 ± 2,312 | 13,973 ± 772 |
| GV-5P | 35,691 ± 159 | 14,457 ± 1,185 | 8,340 ± 725 |
| GV-7 | 38,686 ± 974 | 22,074 ± 3,698 | 15,906 ± 1,687 |
| GV-9 | 30,599 ± 2580 | 15,260 ± 2,764 | 4,531 ± 1,240 |
| GV-13 | 15,296 ± 2,006 | 7,163 ± 833 | 3,701 ± 243 |
| GV-14 | 27,754 ± 1,872 | 13,001 ± 3,273 | 9,897 ± 2,833 |
| GV-22B | 3,199 ± 771 | 3,255 ± 386 | 1,841 ± 318 |
| GV-23 | 35,598 ± 1,330 | 15,423 ± 2,858 | 7,393 ± 2,188 |
| GV-24B | 43,678 ± 2,190 | 30,307 ± 1,533 | 15,375 ± 2,594 |
| GV-27 | 18,165 ± 3,300 | 16,329 ± 1,794 | 6,107 ± 1,773 |
| GV-27A | 23,723 ± 850 | 6,860 ± 746 | 4,295 ± 780 |
| GV-27B | 31,602 ± 1,939 | 29,468 ± 3,867 | 30,306 ± 1,912 |
| GV-29 | 20,034 ± 3,328 | 8,107 ± 488 | 2,982 ± 897 |
| GV-33 | 41,529 ± 1,919 | 27,529 ± 1,238 | 8,764 ± 256 |
| GV-35 | 29,163 ± 2,693 | 9,968 ± 314 | 1,626 ± 406 |
| GV-38AP | 28,971 ± 4,499 | 17,396 ± 878 | 8,060 ± 810 |
| GV-38BP | 19,746 ± 245 | 11,732 ± 3,207 | 6,264 ± 875 |
| GV-40P | 25,185 ± 2,877 | 19,292 ± 2,294 | 10,883 ± 893 |
| GV-41B | 24,646 ± 2,714 | 12,627 ± 3,622 | 5,772 ± 1,041 |
| GV-42 | 25,486 ± 3,029 | 20,591 ± 2,021 | 13,789 ± 775 |
| GV-44 | 2,684 ± 1,995 | 3,577 ± 1,725 | 1,499 ± 959 |
| GV-45 | 9,554 ± 482 | 3,683 ± 1,127 | 1,497 ± 199 |

TABLE 14
Immunogenic properties of GV proteins: IFNγ production

| | IFNγ (ng/ml) | | |
|---|---|---|---|
| | Dose of GV protein used in vitro (μg/ml) | | |
| GV protein | 50 | 10 | 2 |
| GV-1/70 | 24.39 ± 6.66 | 6.19 ± 1.42 | 1.90 ± 0.53 |
| GV-1/83 | 11.34 ± 5.46 | 5.36 ± 1.34 | 2.73 ± 1.55 |
| GV-3 | 3.46 ± 0.30 | 1.57 ± 0.04 | not detectable |
| GV-4P | 6.48 ± 0.37 | 3.00 ± 0.52 | 1.38 ± 0.50 |
| GV-5 | 4.08 ± 1.41 | 6.10 ± 2.72 | 2.35 ± 0.40 |
| GV-5P | 34.98 ± 15.26 | 9.95 ± 3.42 | 5.68 ± 0.79 |
| GV-7 | 33.52 ± 3.08 | 25.47 ± 4.14 | 9.60 ± 1.74 |
| GV-9 | 92.27 ± 45.50 | 88.54 ± 16.48 | 30.46 ± 1.77 |
| GV-13 | 11.60 ± 2.89 | 2.04 ± 0.58 | 1.46 ± 0.62 |
| GV-14 | 8.28 ± 1.56 | 3.19 ± 0.56 | 0.94 ± 0.24 |
| GV-22B | not detectable | not detectable | not detectable |
| GV-23 | 59.67 ± 14.88 | 30.70 ± 4.48 | 9.17 ± 1.51 |
| GV-24B | 6.76 ± 0.58 | 3.20 ± 0.50 | 1.97 ± 0.03 |
| GV-27 | 72.22 ± 11.14 | 30.86 ± 10.55 | 21.38 ± 3.12 |
| GV-27A | 4.25 ± 2.32 | 1.51 ± 0.73 | not detectable |
| GV-27B | 87.98 ± 15.78 | 44.43 ± 8.70 | 21.49 ± 5.60 |
| GV-29 | 7.56 ± 2.58 | 1.22 ± 0.56 | not detectable |
| GV-33 | 7.71 ± 0.26 | 8.44 ± 2.35 | 1.52 ± 0.24 |
| GV-38AP | 23.49 ± 5.89 | 8.87± 1.62 | 4.17 ± 1.72 |
| GV-38BP | 5.30 ± 0.95 | 3.10 ± 1.19 | 1.91 ± 1.01 |
| GV-40P | 15.65 ± 7.89 | 10.58 ± 1.31 | 3.57 ± 1.53 |
| GV-41B | 16.73 ± 1.61 | 5.08 ± 1.08 | 2.13 ± 1.10 |
| GV-42 | 95.97 ± 23.86 | 52.88 ± 5.79 | 30.06 ± 8.94 |
| GV-44 | not detectable | not detectable | not detectable |

B. Activation of Lymphocyte Subpopulations

The ability of recombinant M. vaccae proteins, heat-killed M. vaccae and DD-M. vaccae to activate lymphocyte subpopulations was determined by examining upregulation of expression of CD69 (a surface protein expressed on activated cells).

PBMC from normal donors (5×10⁶ cells/ml) were stimulated with 20 ug/ml of either heat-killed M. vaccae cells, DD-M. vaccae or recombinant GV-22B, GV-23, GV-27, GV27A, GV-27B or GV-45 for 24 hours. CD69 expression was determined by staining cultured cells with monoclonal antibody against CD56, αβT cells or γδT cells in combination with monoclonal antibodies against CD69, followed by flow cytometry analysis

Table 15 shows the percentage of αβT cells, γδT cells and NK cells expressing CD69 following stimulation with heat-killed M. vaccae, DD-M. vaccae or recombinant M. vaccae proteins. These results demonstrate that heat-killed M. vaccae, DD-M. vaccae and GV-23 stimulate the expression of CD69 in the lymphocyte subpopulations tested compared with control (non-stimulated cells), with particularly high levels of CD69 expression being seen in NK cells. GV-45 was found to upregulate CD69 expression in αβT cells.

TABLE 15
Stimulation of CD69 Expression

| | αβT cells | γδT cells | NK cells |
|---|---|---|---|
| Control | 3.8 | 6.2 | 4.8 |
| Heat-killed M. | 8.3 | 10.2 | 40.3 |
| vaccae | | | |
| DD-M. vaccae | 10.1 | 17.5 | 49.9 |
| GV-22B | 5.6 | 3.9 | 8.6 |
| GV-23 | 5.8 | 10.0 | 46.8 |
| GV-27 | 5.5 | 4.4 | 13.3 |
| GV-27A | 5.5 | 4.4 | 13.3 |
| GV-27B | 4.4 | 2.8 | 7.1 |
| GV-45 | 11.7 | 4.9 | 6.3 |

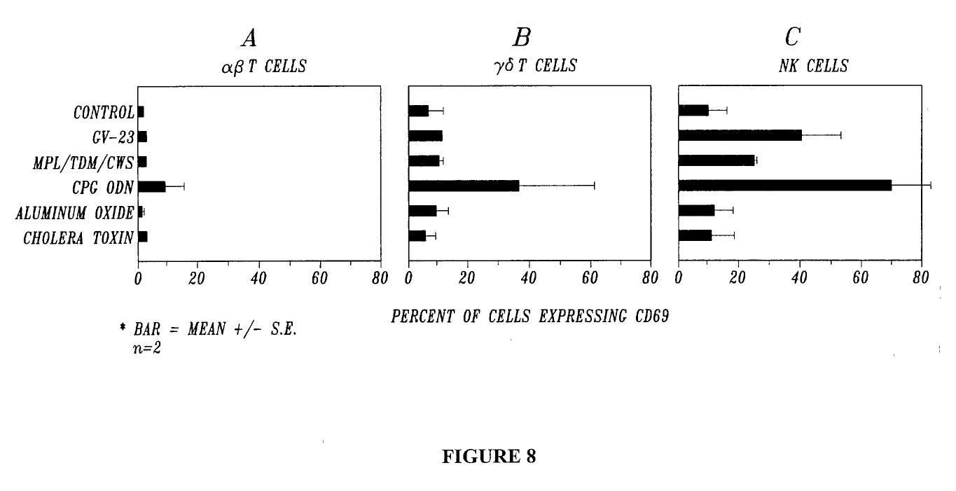

The ability of the recombinant protein GV-23 (20 μg/ml) to induce CD69 expression in lymphocyte subpopulations was compared with that of the known Th1-inducing adjuvants MPL/TDM/CWS (Monophosphoryl Lipid A/Trehalose 6′6′ dimycolate- Sigma, St. Louis, Mo. at a final dilution of 1:20/cell wall skeleton: mycolic acid-arabino-galactan-mucopeptide) and CpG ODN (oligodeoxynucleotide-Promega, Madison, Wis.; 20 μg/ml), and the known Th2-inducing adjuvants aluminium hydroxide (Superfos Biosector, Kvistgard, Denmark; at a final dilution of 1:400) and cholera toxin (20 μg/ml), using the procedure described above. MPL/TDM/CWS and aluminium hydroxide were employed at the maximum concentration that does not cause cell cytotoxicity. FIGS. 8A-C show the stimulation of CD69 expression on αβT cells, γδT cells and NK cells, respectively. GV-23, MPL/TDM/CWS and CpG ODN induced CD69 expression on NK cells, whereas aluminium hydroxide and cholera toxin did not.

C. Stimulation of Cytokine Production

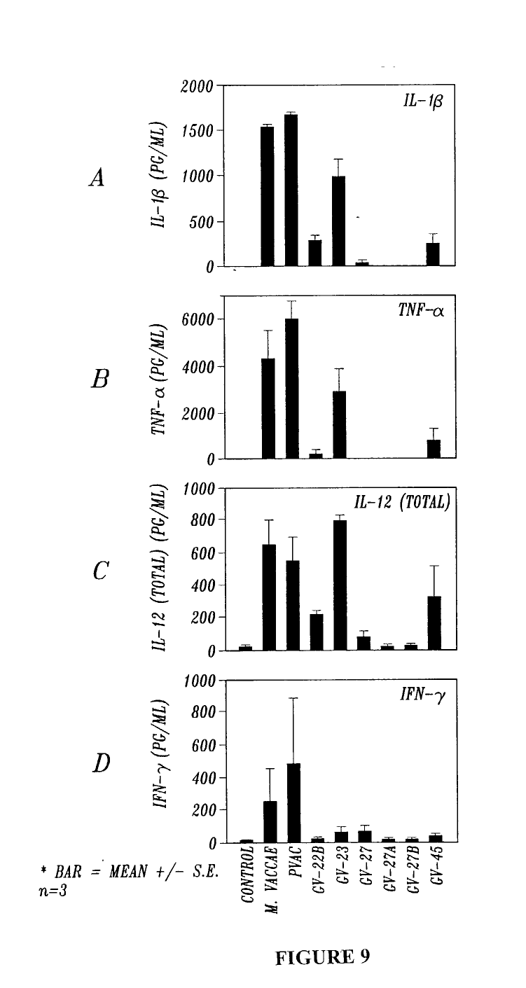

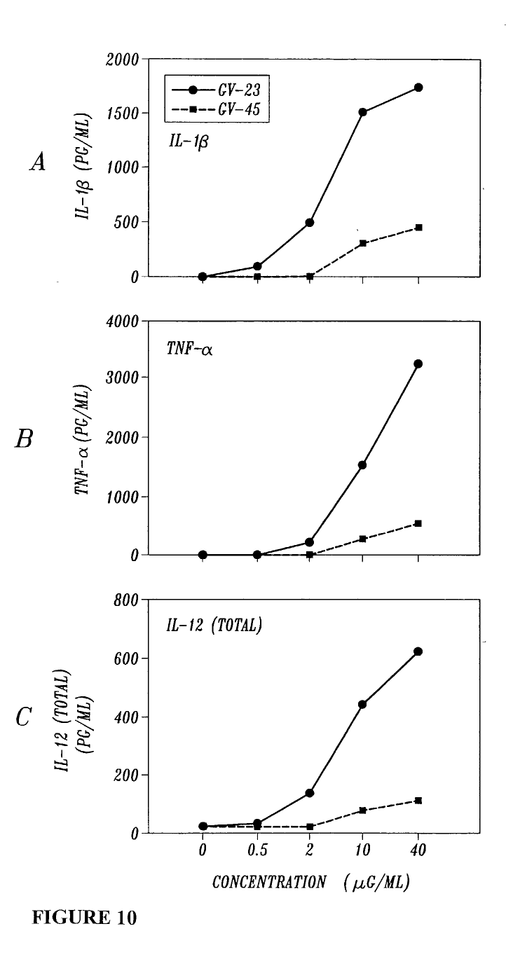

The ability of recombinant M. vaccae proteins to stimulate cytokine production in PBMC was examined as follows. PBMC from normal donors (5×10⁶ cells/ml) were stimulated with 20 ug/ml of either heat-killed M. vaccae cells, DD-M. vaccae, or recombinant GV-22B, GV-23, GV-27, GV27A, GV-27B or GV-45 for 24 hours. Culture supernatants were harvested and tested for the production of IL-1β, TNF-α, IL-12 and IFN-γ using standard ELISA kits (Genzyme, Cambridge, Mass.), following the manufacturer's instructions. FIGS. 9A-D show the stimulation of IL-1β, TNF-α, IL-12 and IFN-γ production, respectively. Heat-killed M. vaccae and DD-M. vaccae were found to stimulate the production of all four cytokines examined, while recombinant GV-23 and GV-45 were found to stimulate the production of IL-1β, TNF-α and IL-12. FIGS. 10A-C show the stimulation of IL-1β, TNF-α and IL-12 production, respectively, in human PBMC (determined as described above) by varying concentrations of GV-23 and GV-45.

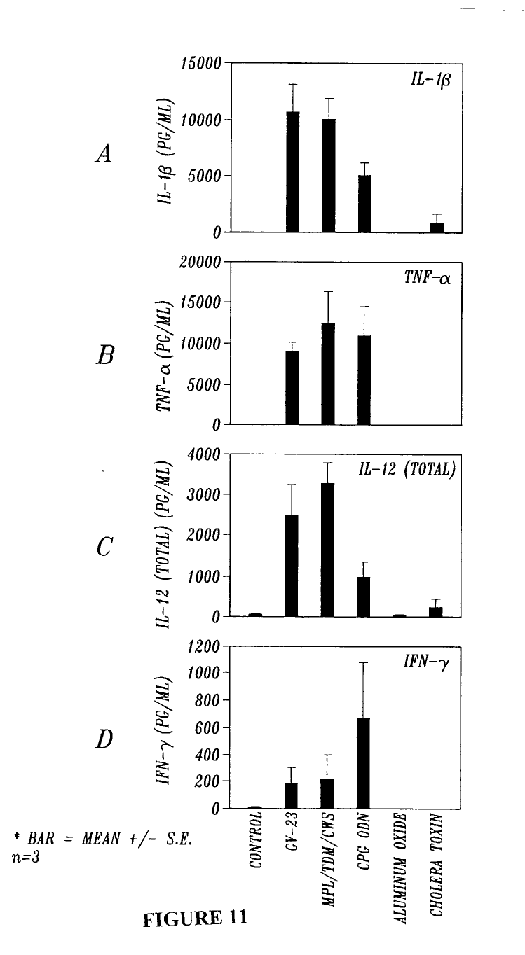

FIGS. 11A-D show the stimulation of IL-1β, TNF-α, IL-12 and IFN-γ production, respectively, in PBMC by GV-23 as compared to that by the adjuvants MPL/TDM/CWS (at a final dilution of 1:20), CpG ODN (20 μg/ml), aluminium hydroxide (at a final dilution of 1:400) and cholera toxin (20 μg/ml). GV-23, MPL/TDM/CWS and CpG ODN induced significant levels of the four cytokines examined, with higher levels of IL-1β production being seen with GV-23 than with any of the known adjuvants. Aluminium hydroxide and cholera toxin induced only negligible amounts of the four cytokines.

D. Activation of Antigen Presenting Cells

The ability of heat-killed M. vaccae, DD-M. vaccae and recombinant M. vaccae proteins to enhance the expression of the co-stimulatory molecules CD40, CD80 and CD86 on B cells, monocytes and dendritic cells was examined as follows.

Peripheral blood mononuclear cells depleted of T cells and comprising mainly B cells, monocytes and dendritic cells were stimulated with 20 ug/ml of either heat-killed M. vaccae cells, DD-M. vaccae, or recombinant GV-22B, GV-23, GV-27, GV27A, GV-27B or GV-45 for 48 hours. Stimulated cells were harvested and analyzed for up-regulation of CD40, CD80 and CD86 using 3 color flow cytometric analysis. Tables 16, 17 and 18 show the fold increase in mean fluorescence intensity from control (non-stimulated cells) for dendritic cells, monocytes, and B cells, respectively.

TABLE 16
Stimulation of CD40, CD80 and CD86
Expression on Dendritic Cells

| | CD40 | CD80 | CD86 |
|---|---|---|---|
| Control | 0 | 0 | 0 |
| Heat-killed M. | 6.1 | 3.8 | 1.6 |
| vaccae | | | |
| DD-M. vaccae | 6.6 | 4.2 | 1.6 |
| GV-22B | 4.6 | 1.9 | 1.6 |
| GV-23 | 6.0 | 4.5 | 1.8 |
| GV-27 | 5.2 | 1.9 | 1.6 |
| GV-27A | 2.3 | 0.9 | 1.0 |
| GV-27B | 2.6 | 1.1 | 1.1 |
| GV-45 | 5.8 | 3.0 | 3.1 |

TABLE 17
Stimulation of CD40, CD80 and CD86 Expression on Monocytes

| | CD40 | CD80 | CD86 |
|---|---|---|---|
| Control | 0 | 0 | 0 |
| Heat-killed M. | 2.3 | 1.8 | 0.7 |
| vaccae | | | |
| DD-M. vaccae | 1.9 | 1.5 | 0.7 |
| GV-22B | 0.7 | 0.9 | 1.1 |
| GV-23 | 2.3 | 1.5 | 0.7 |
| GV-27 | 1.5 | 1.4 | 1.2 |
| GV-27A | 1.4 | 1.4 | 1.4 |
| GV-27B | 1.6 | 1.2 | 1.2 |
| GV-45 | 1.6 | 1.2 | 1.0 |

TABLE 18
Stimulation of CD40, CD80 and CD86 Expression on B Cells

| | CD40 | CD80 | CD86 |
|---|---|---|---|
| Control | 0 | 0 | 0 |
| Heat-killed M. | 1.6 | 1.0 | 1.7 |
| vaccae | | | |
| DD-M. vaccae | 1.5 | 0.9 | 1.7 |
| GV-22B | 1.1 | 0.9 | 1.2 |
| GV-23 | 1.2 | 1.1 | 1.4 |
| GV-27 | 1.1 | 0.9 | 1.1 |
| GV-27A | 1.0 | 1.1 | 0.9 |
| GV-27B | 1.0 | 0.9 | 0.9 |
| GV-45 | 1.2 | 1.1 | 1.3 |

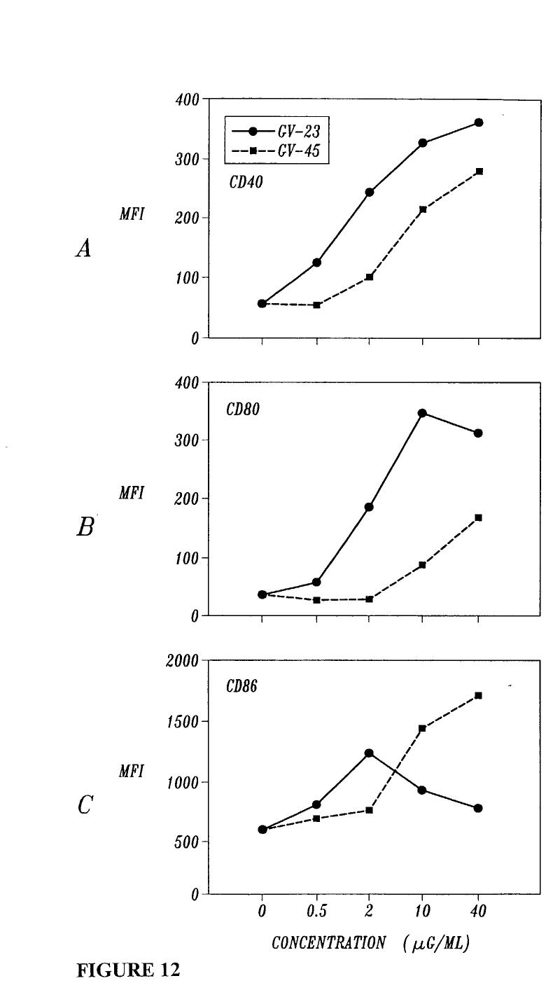

As shown above, increased levels of CD40, CD80 and CD86 expression were seen in dendritic cells, monocytes and B cells with all the compositions tested. Expression levels were most increased in dendritic cells, with the highest levels of expression being obtained with heat-killed M. vaccae, DD-M. vaccae, GV-23 and GV-45. FIGS. 12A-C show the stimulation of expression of CD40, CD80 and CD86, respectively, in dendritic cells by varying concentrations of GV-23 and GV-45.

The ability of GV-23 to stimulate CD40, CD80 and CD86 expression in dendritic cells was compared to that of the Th1-inducing adjuvants MPL/TDM/CWS (at a final dilution of 1:20) and CpG ODN (20 μg/ml), and the known Th2-inducing adjuvants aluminium hydroxide (at a final dilution of 1:400) and cholera toxin (20 μg/ml). GV23, MPL/TDM/CWS and CpG ODN caused significant up-regulation of CD40, CD80 and CD86, whereas cholera toxin and aluminium hydroxide induced modest or negligible dendritic cell activation, respectively.

E. Dendritic Cell Maturation and Function

The effect of the recombinant M. vaccae protein GV-23 on the maturation and function of dendritic cells was examined as follows.

Purified dendritic cells (5×10⁴−10⁵ cells/ml) were stimulated with GV-23 (20 μg/ml) or LPS (10 μg/ml) as a positive control. Cells were cultured for 20 hour and then analyzed for CD83 (a maturation marker) and CD80 expression by flow cytometry. Non-stimulated cells were used as a negative control. The results are shown below in Table 19.

TABLE 19
Stimulation of CD83 Expression in Dendritic Cells

| | % CD83-positive | % CD80-positive |
|---|---|---|
| Treatments | dendritic cells | dendritic cells |
| Control | 15 ± 8 | 9 ± 6.6 |
| GV-23 | 35 ± 13.2 | 24.7 ± 14.2 |
| LPS | 36.3 ± 14.8 | 27.7 ± 13 |

Data = mean ± SD (n = 3)

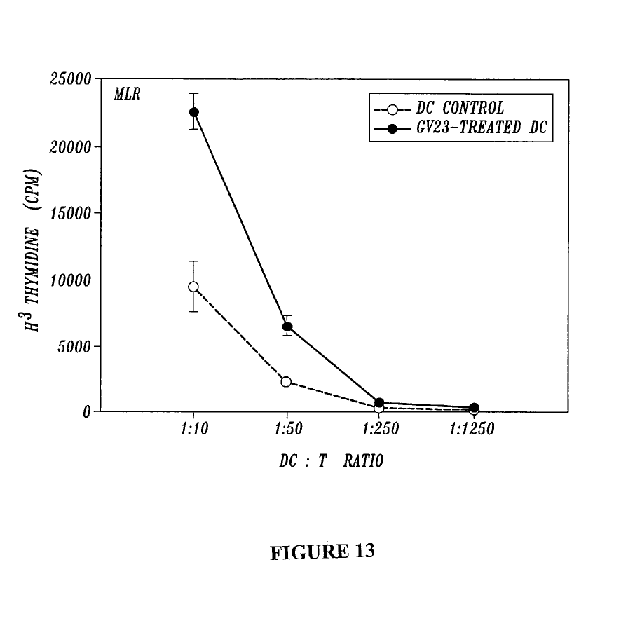

The ability of GV-23 to enhance dendritic cell function as antigen presenting cells was determined by mixed lymphocyte reaction (MLR) assay. Purified dendritic cells were cultured in medium alone or with GV-23 (20 μg/ml) for 18-20 hours and then stimulated with allogeneic T cells (2×10⁵ cells/well). After 3 days of incubation, (³H)-thymidine was added. Cells were harvested 1 day later and the uptake of radioactivity was measured. FIG. 13 shows the increase in uptake of (³H)-thymidine with increase in the ratio of dendritic cells to T cells. Significantly higher levels of radioactivity uptake were seen in GV-23 stimulated dendritic cells compared to non-stimulated cells, showing that GV-23 enhances dendritic cell mixed lymphocyte reaction.

### EXAMPLE 11

### Effect of Intraperitoneal Administration of AVAC on the Expression of Genes Involved in Notch Signaling in Mice

The capacity of AVAC to modulate expression of genes involved in Notch signaling was assessed in 6-week-old female BALB/cByJ mice as follows. On day 0, mice were immunized intraperitoneally (i.p.) with a mixture containing 10 μg ovalbumin adsorbed to 1 mg aluminium hydroxide adjuvant (Alum, Alu-Gel-S, Serva), or with OVA-Alum mixture to which was added 1 mg AVAC, using 10 mice per group. On day 7, all mice were immunized i.p. with OVA-Alum only. Ten days later, all mice were sacrificed. The spleen was removed from each animal, pooled with other spleens from the same treatment group, and cell suspensions prepared. CD4⁺ cells were isolated from each pooled spleen cell suspension using a Mouse T Cell CD4 Subset Kit (R&D Systems, Minneapolis Minn.). The cells, >75% CD4+ as determined by flow cytometry using FITC-conjugated rat anti-mouse CD4 monoclonal antibody (clone GK1.5, Pharmingen), were then stored in TRIZOL™ (Invitrogen) at −80° C. RNA was extracted as per the manufacturer's instructions, and 1 μg of purified RNA was transcribed into cDNA using Superscript (Invitrogen), and subjected to real-time PCR analysis using an ABI Prism 7700 Sequence Detection System (Perkin Elmer/Applied Biosystems, Foster City, Calif.). Primers and fluorogenic probes were specific for human Notch1, Notch2, Notch3, Delta1, Delta3, Serrate1, Serrate2, HES1, HES5, and Deltex.

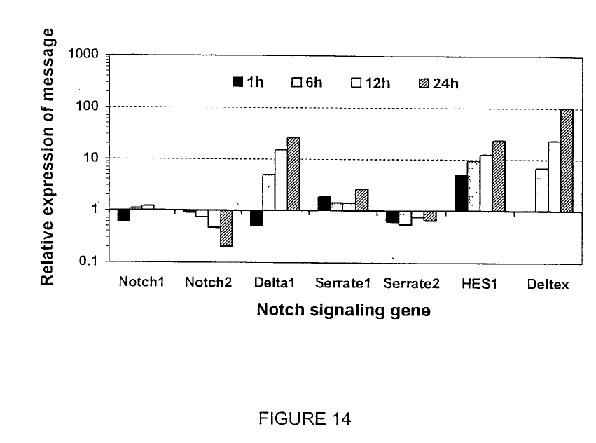

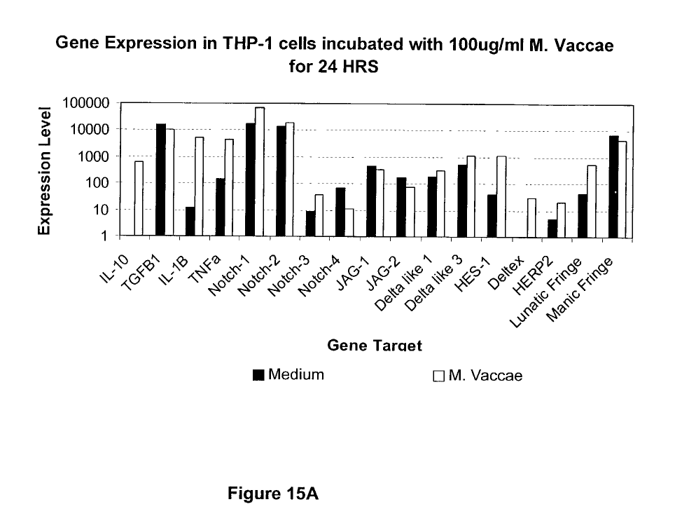

As shown in FIG. 14, real-time PCR analysis revealed that treatment of mice with AVAC caused striking increases in expression of Notch receptors, ligands, and downstream targets. Relative expression of Notch receptors ranged from 8-fold (Notch3) up to 22-fold (Notch1). With the exception of Delta1 (<2-fold), relative expression of Notch ligands ranged from almost 15-fold (Delta3, Serrate2) to >100-fold (Serrate1). Relative, expression of downstream Notch signaling targets ranged from 2-fold (HES1) to 6-fold (Deltex).

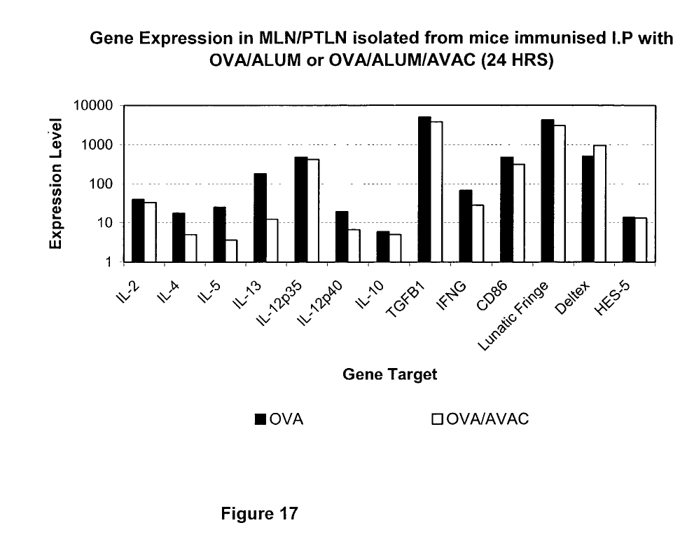

In subsequent experiments, the ability of AVAC to modulate expression of the Notch signaling genes HES5, Lunatic Fringe and Deltex, as well as the cytokines IL-2, IL-4, IL-5, IL-13, IL-12p35, IL-12p40, IL-10, TGFbeta1, IFN-gamma and CD86, as examined essentially as described above. As shown in FIG. 17, real-time PCR analysis revealed that treatment of mice with AVAC caused suppression of IL-4 (3.5 fold), IL-5 (7 fold) and IL-13 (15 fold) gene expression. These gene products are required for allergic sensitization and are Th2 type cytokines.

### EXAMPLE 12

### Effect of Intranasal Administration of AVAC and DD-M. vaccae on Expression of Genes Involved in Notch Signaling in Mice

The ability of DD-M. vaccae and AVAC to modulate expression of genes involved in Notch signaling was assessed in 6-week-old female BALB/cByJ mice as follows.

Three mice per group were immunized intranasally with 50 μl PBS containing 1 mg AVAC or 1 mg DD-M. vaccae. Mice were sacrificed 24 hours later and lung samples from the mice were snap-frozen in liquid nitrogen for RNA extraction. Samples from individual animals were pooled into treatment groups and lung tissues were homogenized. Total RNA was extracted using Trizol reagent, 1 μg of purified RNA transcribed into cDNA using Superscript First Strand Synthesis System (Invitrogen), and subjected to real-time PCR analysis using an ABI Prism 7700 Sequence Detection System (Perkin Elmer/Applied Biosystems, Foster City, Calif.). Primers and fluorogenic probes were specific for human Notch1, Notch2, Notch3, Notch4, Delta4, HES5 and Deltex, as well as the cytokines TGFbeta1, IL-2 and IL-10.

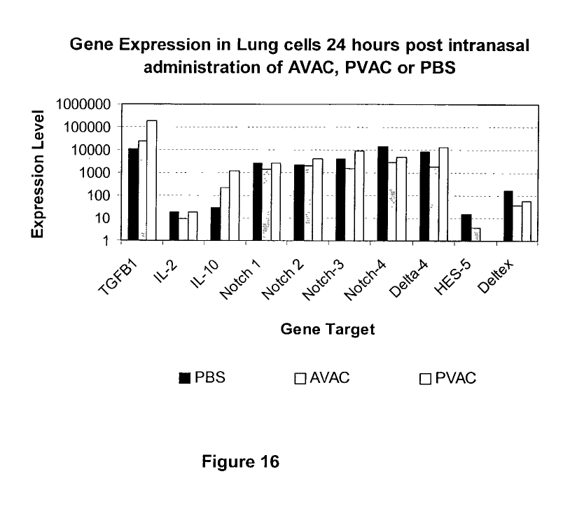

As shown in FIG. 16, real-time PCR analysis revealed that treatment of mice with AVAC and DD-M. vaccae (referred to as PVAC in FIG. 16) caused TGFβ1 gene expression to be significantly induced in comparison to the control group. Significant IL-10 gene induction was also found in both treatment groups. TGFβ1 and IL-10 are considered to be anti-inflammatory. HES-5 gene expression was suppressed in the AVAC treated group (˜4 fold) and was not detectable in the DD-M. vaccae treated group. Deltex gene expression was suppressed in the presence of AVAC and DD-M. vaccae.

### EXAMPLE 13

### Effect of M. vaccae, DD-M. vaccae, AVAC and M. vaccae Glycolipids on Expression of Cytokines and Genes Involved in Notch Signaling in Human Cells

The ability of inactivated M. vaccae, DD-M. vaccae, AVAC and M. vaccae glycolipids to modulate expression of genes involved in Notch signaling, cytokines and Toll-like receptors (TLR) was assessed as follows using the human myelomonocytic cell line THP-1 (American Type Culture Collection, Manassas, Va.).

THP-1 cells were maintained in RPMI (Gibco BRL Life Technologies) supplemented with antibiotics, L-glutamine, 2-mercaptoethanol, and 5% fetal calf serum (cRPMI-5). For assay, THP-1 cells were resuspended at 1×10⁶/ml in cRPMI-5 in a volume of 4 ml in 6-well plates. After saving an aliquot of THP-1 cells for reference purposes (t=0 hr baseline control), inactivated M. vaccae, DD-M. vaccae, AVAC or M. vaccae glycolipids was added to the cell suspension to achieve a final concentration of 100 μg/ml. The cells were subsequently cultured in a humidified 37° C. incubator supplied with a gas mixture of 5% CO₂ in air. Cells were collected at various time points (3, 6, 12 and 24 hours), centrifuged, resuspended in TRIZOL™ (Gibco BRL Life Technologies), and frozen at −80° C. RNA was extracted as per the manufacturer's instructions, and 1 μg of purified RNA was transcribed into cDNA using Superscript First Strand Synthesis System (Invitrogen, Carlsbad, Calif.), and the cDNA subjected to real-time PCR analysis using an ABI Prism 7700 Sequence Detection System (Perkin Elmer/Applied Biosystems, Foster City, Calif.). Primers and fluorogenic probes were specific for the Notch signaling genes human Notch1, Notch2, Notch3, Notch4, Deltex, Jagged-1, Jagged-2, Delta-like 1, Delta-like 3, HES-1, HERP1, HERP2, Lunatic Fringe, Manic Fringe, Radical Fringe, Numb, MAML1 and RBP-Jkappa; the Toll-like receptors TLR2, TLR7, TLR8, MyD88 and CD14; and the cytokines IL-12p35, IL-12p40, IL-10, IL-1β, IL-6, IL-8, IL-23p19 and TNFα.

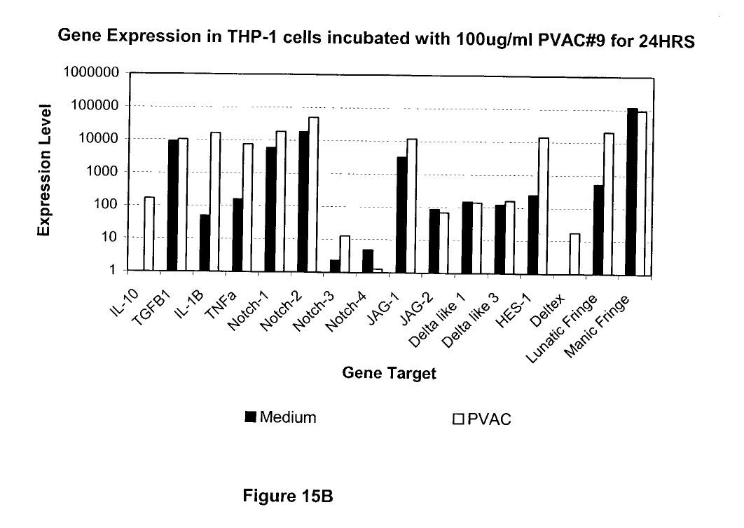

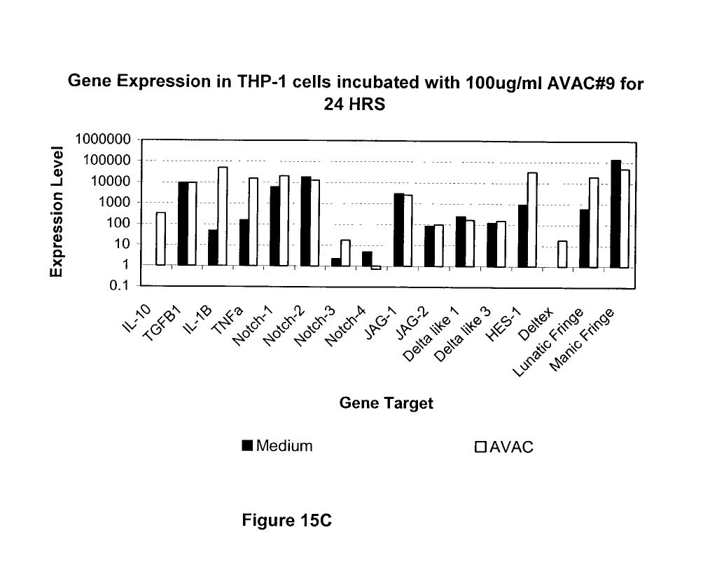

As shown in FIG. 15A-C, IL-10, IL-1β and TNFα gene expression was dramatically upregulated in response to all stimuli. The Notch related genes Lunatic Fringe and HES-1 were dramatically induced (˜30 fold) with stimuli showing a dose/response and time dependent induction of Lunatic Fringe and HES-1 gene expression. Deltex gene expression was also upregulated by these stimuli but was below detection limits in the absence of stimuli. There was a trend towards Notch-1 (3-4 fold) and Notch-3 (2.5-8 fold) upregulation and Notch 4 downregulation (−3 to −7 fold).

Table 20 summarizes the effects of inactivated M. vaccae, DD-M. vaccae, AVAC, and M. vaccae glycolipids on the expression of genes involved in Notch signaling in THP-1 cells.

TABLE 20

| | Relative expression* | | | | |
|---|---|---|---|---|---|
| Notch signaling gene | M. vaccae | DD-M. vaccae | AVAC | Glycolipids | LPS |
| Notch1 | 1.90 | 1.60 | 3.20 | 1.90 | 2.30 |
| Notch2 | 1.40 | 1.10 | 1.40 | 1.20 | 1.40 |
| Notch3 | 5.00 | — | 15.1 | 1.90 | 2.30 |
| Notch4 | 0.06 | 0.16 | 0.14 | 0.24 | 0.10 |
| Jagged1 | 1.80 | 1.30 | 1.10 | 2.20 | 1.70 |
| Jagged2 | 0.31 | 0.90 | 0.90 | 0.34 | 0.54 |
| Delta1 | 7.20 | 1.20 | 2.50 | 0.90 | 0.80 |
| Delta-like3 | 0.47 | 1.20 | 1.00 | 1.50 | 1.20 |
| Delta-like4 | 134.8 | 64.6 | 46.4 | 25.5 | 41.6 |
| HES1 | 57.0 | 71.0 | 140.0 | 22.0 | 49.0 |
| Deltex | 7.00 | 5.50 | 11.70 | 2.70 | 1.00 |
| HERP1 | — | — | — | — | — |
| HERP2 | 7.00 | 2.30 | 4.50 | 0.69 | 1.00 |
| Lunatic fringe | 12.0 | 9.00 | 18.0 | 7.50 | 4.00 |
| Manic fringe | 0.38 | 0.67 | 0.30 | 0.59 | 0.45 |
| Radical fringe | 0.65 | 0.89 | 0.92 | 0.80 | 0.67 |
| Presenilin1 | 1.39 | 1.37 | 0.85 | 1.54 | 1.28 |
| Numb | 1.89 | 1.29 | 1.26 | 0.92 | 0.74 |
| MAML1 | 1.06 | 1.27 | 0.90 | 0.96 | 0.67 |
| RBP-Jκ | 0.78 | 1.21 | 0.94 | 0.62 | 0.56 |
| HASH1 | 0.16 | 0.23 | 0.31 | 0.15 | 1.00 |

*Normalized relative expression of target gene mRNA in stimulus vs. medium control samples at t = 24 hr.

As shown in Table 20, M. vaccae upregulated Notch3, Delta1, Delta-like4, HES1, Deltex, HERP2, and Lunatic fringe expression; DD-M. vaccae upregulated Delta-like4, HES1, Deltex and Lunatic fringe expression; AVAC upregulated Notch1, Notch3, (Delta1), Delta-like4, HES1, Deltex, HERP2 and Lunatic fringe expression; and M. vaccae glycolipids upregulated Delta-like4, HES1, Deltex and Lunatic fringe expression. M. vaccae down-regulated Notch4, Jagged2, Manic fringe and HASH1 expression; DD-M. vaccae down-regulated Notch4 and HASH1; AVAC down-regulated Notch4, Manic fringe and HASH1 expression and M. vaccae glycolipids down-regulated Notch4, Jagged2 and HASH1 expression.

A summary of the effects of inactivated M. vaccae, DD-M. vaccae, AVAC, and M. vaccae glycolipids on the expression of cytokines in THP-1 cells is presented in Table 21.

TABLE 21

| | Relative expression* | | | | |
|---|---|---|---|---|---|
| Cytokine gene | M. vaccae | DD-M. vaccae | AVAC | Glycolipids | LPS |
| IL-1β | 4939 | 1097 | 2759 | 4011 | 246 |
| IL-6 | 260 | 125 | 130 | 11.6 | 27.1 |
| IL-8 | 3769 | 695 | 1722 | 284 | 267 |
| IL-10 | 391 | 17.6 | 47.5 | 11.2 | 8.6 |
| IL-12p35 | 0.21 | 0.08 | 0.10 | 0.05 | 0.19 |
| IL-12p40 | 576 | 14.8 | 2684 | 115 | 311 |
| IL-23p19 | 198 | 93.0 | 252 | 18.0 | 8.0 |
| TNFα | 10.3 | 4.1 | 5.3 | 4.7 | 5.7 |

*Normalized relative expression of target gene mRNA in stimulus vs. medium control samples at t = 24 hr.

As shown in Table 21, M. vaccae upregulated IL-1β, IL-6, IL-8, IL-10, IL-12p40, IL-23p19 and TNFα expression; DD-M. vaccae upregulated IL-1β, IL-6, IL-8, IL-10, IL-12p40, IL-23p19 and TNFα expression; AVAC upregulated IL-1β, IL-6, IL-8, IL-10, IL-12p40, IL-23p19 and TNFα expression; and M. vaccae glycolipids upregulated IL-1β, IL-6, IL-8, IL-10, IL-12p40, IL-23p19 and TNFα expression. M. vaccae downregulated IL-12p35; DD-M. vaccae downregulated IL-12p35; AVAC downregulated IL-12p35; and M. vaccae glycolipids downregulated IL-12p35 expression.

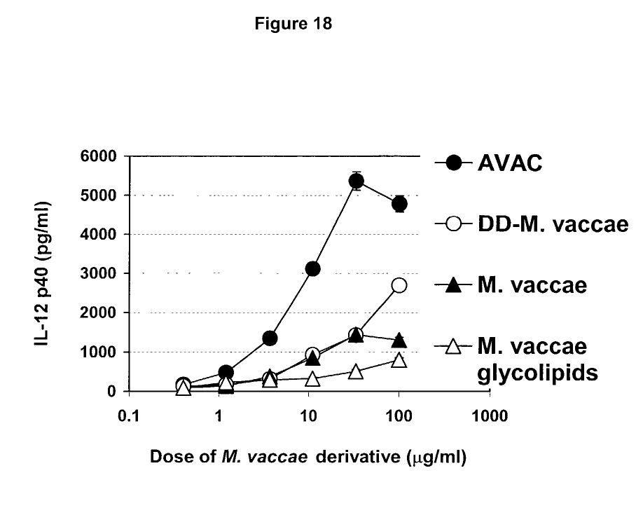

In further studies, the production of IL-12p40 protein in THP-1 cells in response to increasing concentrations of heat-killed M. vaccae, DD-M. vaccae, AVAC and M. vaccae glycolipids was examined by ELISA as described above. As shown in FIG. 18, production of IL-12p40 was found to increase with increasing concentrations of M. vaccae derivatives.

The differential effect of M. vaccae derivatives on IL-12 and IL-23 gene expression in THP-1 cells was examined using real-time PCR as follows.

THP-1 cells were maintained in RPMI (Gibco BRL Life Technologies) supplemented with antibiotics, L-glutamine, 2-mercaptoethanol, and 5% fetal calf serum (cRPMI-5). THP-1 cells were cultured with 100 μg/mL heat-killed M. vaccae, 100 μg/mL DD-M. vaccae, 100 μg/mL AVAC, with M. vaccae glycolipids, or with no M. vaccae derivative for 24 hours in cell culture medium in 6-well tissue culture plates at 1×10⁶ cells/mL in a final volume of 4.0 mL cRPMI-10 (or 4×10⁶ cells per well) in a water-jacketed, humidified incubator at 37° C. and supplied with 5% CO₂ in air. At the end of the 24-hour incubation period, the cells were collected and centrifuged at 200×g for 5 minutes, and the supernatants transferred to sterile 10-ml tubes. 1.0 ml Trizol Reagent (Gibco cat. no. 15596-018) were added to each well to lyse the cells. The resulting mixture in each well was then transferred to a sterile 1.8-ml cyrovial and stored at −80° C.

Isolation of RNA for synthesis of cDNA was performed as described in the protocol supplied with the Trizol Reagent. RNA isolated as above was treated with DNasel (1 U/mL, Invitrogen cat. no. 18008-015). Synthesis of cDNA was then performed as described in the protocol supplied with the First Strand CDNA Synthesis Kit (Invitrogen cat. no. 11904-018).

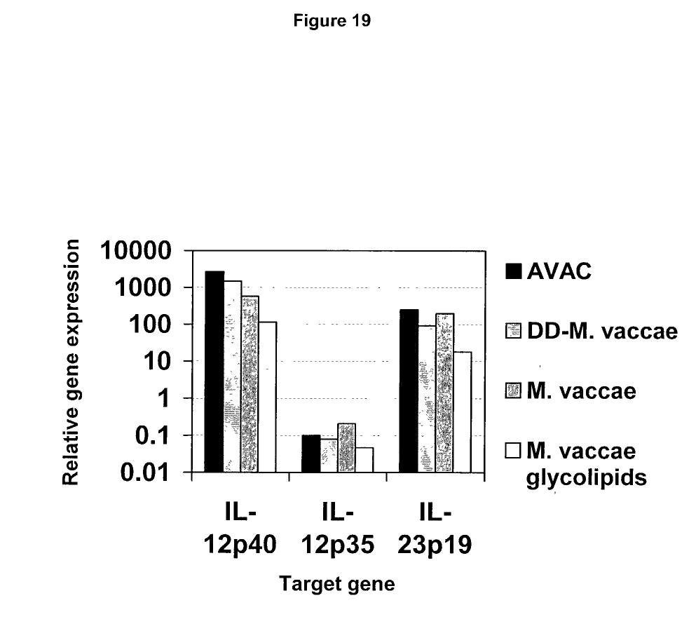

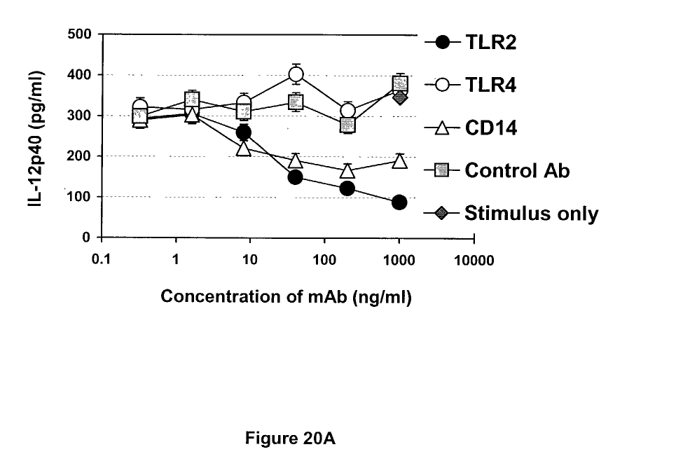

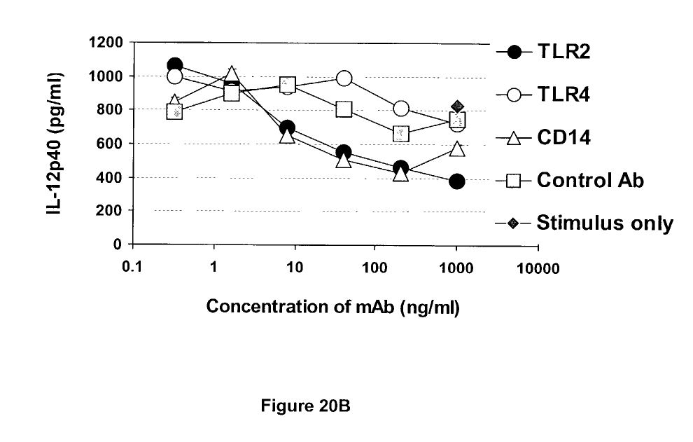

Forward and reverse primers were designed using Perkin Elmer/Applied Biosystems (ABI) Primer Express software. Real-time PCR was performed using methodology reported by Lin Yin et al (Immunol Cell Biol 79:213-221, 2001) and amplification curves plotted using the ABI 7700 Sequence Detection System (Perkin Elmer/Applied Biosystems). Expression data obtained for THP-1 cells cultured with M. vaccae derivatives were normalized to levels observed for THP-1 cells cultured in cRPMI-10 only, and the normalized values plotted as relative expression levels. As shown in FIG. 19, AVAC, DD-M. vaccae, heat-killed M. vaccae and M. vaccae glycolipids were shown to induce expression of IL-12p40 and IL-23p19 mRNA and to suppress expression of IL-12p35 mRNA.

### EXAMPLE 14

### Effect of M. vaccae, DD-M. vaccae, AVAC and M. vaccae Glycolipids on Toll-Like Receptor Signaling in Human Cells

Since the Toll-like receptor TLR2 is known to mediate biological effects of mycobacteria and their products, particularly cell wall components, and since DD-M. vaccae and AVAC contain at least one known TLR2 ligand, namely peptidoglycan, the effect of M. vaccae derivatives on the expression of TLR genes in THP-1 cells was examined essentially as described above using primers and fluorogenic probes specific for the TLR signaling genes CD14, TLR2, TLR7, TLR8 and MyD88. A summary of the effects of inactivated M. vaccae, DD-M. vaccae, AVAC, and M. vaccae glycolipids on TLR signaling in THP-1 cells is presented in Table 22.

TABLE 22

| | Relative expression* | | | | |
|---|---|---|---|---|---|
| TLR signaling gene | M. vaccae | DD-M. vaccae | AVAC | Glycolipids | LPS |
| CD14 | 44.5 | 48.6 | 68.3 | 26.7 | 16.3 |
| TLR2 | 1.9 | 2.0 | 1.0 | 1.7 | 1.7 |
| TLR7 | 2.0 | 5.5 | 1.7 | 11.4 | 4.2 |
| TLR8 | 42.6 | 77.2 | 133.4 | 67.6 | 42.1 |
| MyD88 | 3.2 | 2.5 | 1.6 | 1.1 | 3.3 |

*Normalized relative expression of target gene mRNA in stimulus vs. medium control samples at t = 24 hr.

These results demonstrate that M. vaccae upregulated CD14 and MyD88 expression; DD-M. vaccae upregulated CD14, TLR7 and TLR8 expression; AVAC upregulated CD14, TLR8 expression; and M. vaccae glycolipids upregulated CD14, TLR7 and TLR8 expression.

In subsequent experiments, the effect of antibodies to TLR2, TLR4 and CD14 on the production of IL-12p40, IL-10 and TNF-α in THP-1 cells in response to M. vaccae derivatives was examined as follows.

THP-1 cells were maintained in RPMI (Gibco BRL Life Technologies) supplemented with antibiotics, L-glutamine, 2-mercaptoethanol, and 5% fetal calf serum (cRPMI-5). Prior to culture with M. vaccae derivatives, 50 μL of THP-1 cells in cRPMI-10 were pre-treated in duplicate microplate wells with 50 μL of serially diluted Functional Grade mabs to human TLR2 (clone TL2.1, IgG2a isotype, eBioscience cat. no. 16-9922-82), TLR4 (clone HTA125, IgG2a isotype, eBioscience cat. no. 16-9927-82), or CD14 (clone RM052, IgG2a isotype, Coulter cat. no. IM0643), with a cocktail of all three antibodies or with control mAb (clone AcV1, IgG2a isotype, eBioscience cat. no. 16-4724-85), with each mAb used at a final concentration of 1000 μg/mL, 200 μg/mL, 40 μg/mL, 8.0 μg/mL, 1.60 μg/mL, or 0.32 μg/mL, or with no mAb. Pretreatment of cells with mAbs was for 60 minutes in a water-jacketed, humidified incubator at 37° C. supplied with 5% CO₂ in air.

Following pretreatment with mAbs, THP-1 cells were cultured with 5 μg/mL heat-killed M. vaccae (MV), 5 μg/mL DD-M. vaccae, 5 μg/mL AVAC, or with no M. vaccae derivative for 24 hours in cell culture medium in 96-well round-bottom microculture plates at 1×10⁶ cells/mL in a final volume of 0.2 mL cRPMI-10 (or 2×10⁵ cells per microwell) in a water-jacketed, humidified incubator at 37° C. and supplied with 5% CO₂ in air. At the end of the 24-hour incubation period, the microplates were centrifuged at 200×g for 5 minutes and the supernatants collected and transferred to a sterile 96-well round-bottom plate.

IL-12p40, TNFα, and IL-10 content in the microculture supernatants was determined by sandwich ELISA using commercially available sets according to the manufacturer's recommendations. For IL-12p40, supernatants were diluted 1:2 in cRPMI-10 prior to analysis and the sensitivity of the ELISA was 4 pg IL-12p40 per mL. For TNFα, supernatants were diluted 1:5 in cRPMI-10 prior to analysis and the sensitivity of the ELISA was 8.0 pg TNFα per mL. For IL-10, supernatants were diluted 1:2 in cRPMI-10 prior to analysis and the sensitivity of the ELISA was 2.0 pg IL-10 per mL.

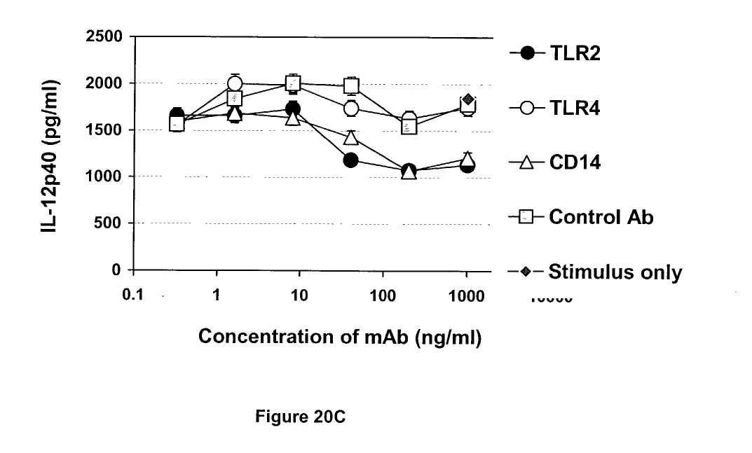

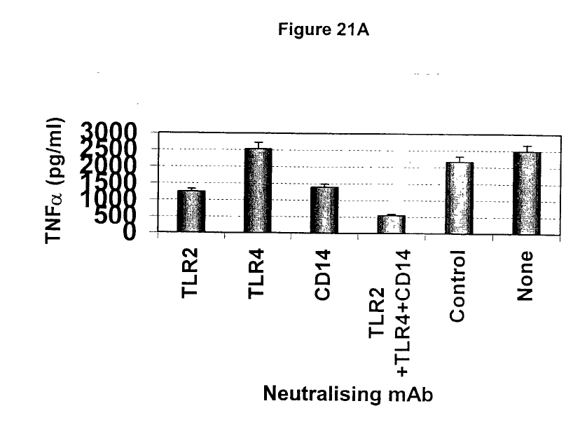

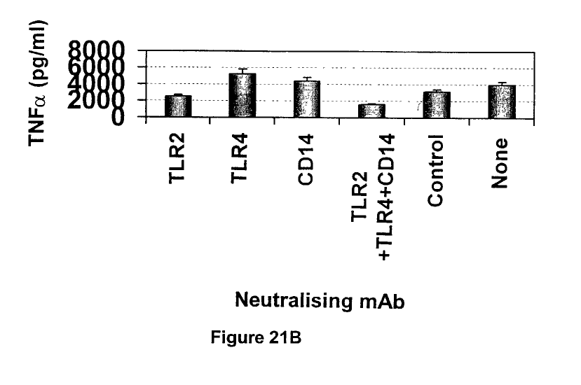

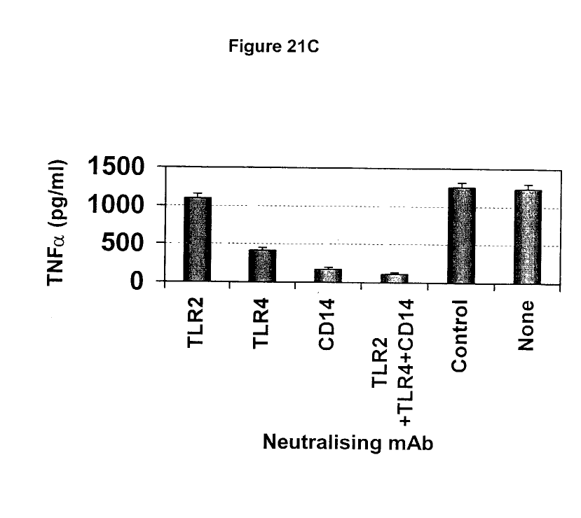

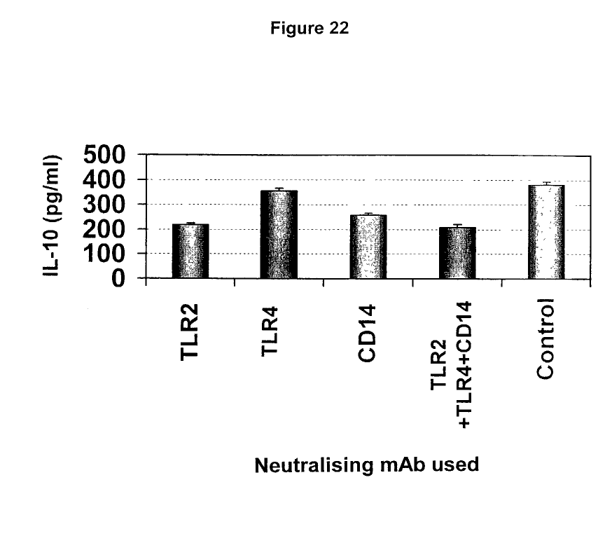

The production of IL-12p40 by THP-1 cells cultured with neutralizing antibodies and either heat-killed M. vaccae, DD-M. vaccae or AVAC is shown in FIGS. 20A-C, respectively. These figures show that M. vaccae-, AVAC- and DD-M. vaccae-induced production of IL-12p40 is inhibited by TLR2 and CD14 mAbs in a dose-dependent fashion. The production of TNFα by THP-1 cells cultured with neutralizing antibodies and either heat-killed M. vaccae, DD-M. vaccae or LPS is shown in FIGS. 21A-C, respectively. FIG. 22 shows the production of IL-10 by THP-1 cells cultured with neutralizing antibodies and heat-killed M. vaccae. These results provide evidence that M. vaccae derivatives elicit production of cytokines through Toll-like receptor signaling.

### EXAMPLE 15

### Effect of M. vaccae, DD-M. vaccae, AVAC and M. vaccae Glycolipids on MRP8 Signaling in Human Cells

The effect of M. vaccae derivatives on MRP8 (S100A8) signaling in THP-1 cells was determined essentially as described above using primers and fluorogenic probes for MRP8. The results are shown in Table 23.

TABLE 23
Relative expression of MRP8

| M. vaccae | DD-M vaccae | AVAC | Glycolipids | LPS |
|---|---|---|---|---|
| 44.5 | 48.6 | 68.3 | 26.7 | 16.3 |

*Normalized relative expression of MRP8 gene mRNA in stimulus vs. medium control samples at t = 24 hr.

These results demonstrate that M. vaccae, DD-M. vaccae, AVAC, M. vaccae glycolipids all upregulate expression of MRP8 (S100A8). MRP-8 is a calcium-binding protein associated with psoriasis and other inflammatory skin disorders. A causal relationship between MRP-8 expression and disease has not yet been established.

### EXAMPLE 16

### Involvement of MAP Kinase Signaling in Production of Cytokines in Human Cells in Response to AVAC

The involvement of the MAP kinase signaling pathway in the production of IL-10 by THP-1 cells in response to AVAC was assessed as follows.

THP-1 cells were maintained in RPMI (Gibco BRL Life Technologies) supplemented with antibiotics, L-glutamine, 2-mercaptoethanol, and 5% fetal calf serum (cRPMI-5). Prior to culture with AVAC, 50 μL of THP-1 cells in cRPMI-10 were pre-treated in duplicate microplate wells with 50 μL of serially diluted PD98059 (Calbiochem cat. no. 51300, a selective inhibitor of MAP kinase), SB202190 (Calbiochem cat. no. 559388, an inhibitor of p38 MAP kinase and p38β MAP kinase), SB203580 (Calbiochem cat. no. 559389, a highly specific inhibitor of p38 MAP kinase), with SB202474 (Calbiochem cat. no. 559387, a negative control for MAP kinase inhibition studies), or with no added chemicals. MAP kinase inhibitors and control were used at a final concentration of 100 μg/mL, 20 μg/mL, 4.0 μg/mL, 0.8 μg/mL, 0.16 μg/mL, or 0.032 μM. Pretreatment of cells with MAP kinase inhibitors and control was for 120 minutes in a water-jacketed, humidified incubator at 37° C. supplied with 5% CO₂ in air.

Following pretreatment, the cells were washed once in cPRMI-10 to remove inhibitor or control chemicals. The THP-1 cells were then cultured with 25 μg/mL AVAC, or with no M. vaccae derivative for 24 hours in cell culture medium in 96-well round-bottom microculture plates at 1×10⁶ cells/mL in a final volume of 0.2 mL cRPMI-10 (or 2×10⁵ cells per microwell) in a water-jacketed, humidified incubator at 37° C. and supplied with 5% CO₂ in air. At the end of the 24-hour incubation period, the microplates were centrifuged at 200×g for 5 minutes and the supernatants collected and transferred to a sterile 96-well round-bottom plate. IL-10 content in the microculture supernatants was determined by sandwich ELISA using a commercially available set (eBioscience cat. no. 88-7106-77,) according to the manufacturer's recommendations. Supernatants were diluted 1:2 in cRPMI-10 prior to analysis. The sensitivity of the ELISA was approximately 2.0 pg IL-10 per mL.

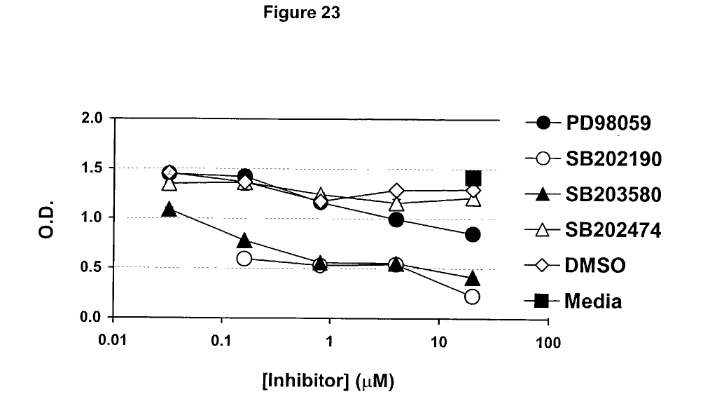

The results of this experiment, expressed in Optical Density (O.D.) values are provided in FIG. 23, and show that production of IL-10 by THP-1 cells cultured with AVAC was substantially suppressed in a dose-dependent manner by the p38 MAP kinase inhibitors SB202190 and SB203580, and to a lesser extent by the MAP kinase inhibitor PD98059. These data indicate that production of IL-10 by THP-1 cells in response to AVAC involves the MAP kinase signaling pathway.

Although the present invention has been described in some detail by way of illustration and example for purposes of clarity of understanding, changes and modifications can be carried out without departing from the scope of the invention which is intended to be limited only by the scope of the appended claims.

We claim:

1. A method for modulating the expression of Notch ligands on antigen presenting cells, comprising contacting the antigen presenting cells with a composition comprising at least one component selected from the group consisting of:
(a) inactivated M. vaccae cells;
(b) delipidated and deglycolipidated M. vaccae cells;
(c) delipidated and deglycolipidated M. vaccae cells that have been treated by alkaline hydrolysis;
(d) delipidated and deglycolipidated M. vaccae cells that have been treated by acid hydrolysis;
(e) delipidated and deglycolipidated M. vaccae cells that have been treated with periodic acid;
(f) delipidated and deglycolipidated M. vaccae cells that have been treated by alkaline hydrolysis and by acid hydrolysis;
(g) delipidated and deglycolipidated M. vaccae cells that have been treated by alkaline hydrolysis and treated with periodic acid;
(h) delipidated and deglycolipidated M. vaccae cells that have been treated with Proteinase K; and
(i) delipidated and deglycolipidated M. vaccae cells that have been treated by hydrofluoric acid hydrolysis.

2. The method of claim 1, wherein the antigen presenting cells are dendritic cells.

3. A method for modifying an immune response to an antigen in a subject, comprising administering to the subject a composition comprising at least one component selected from the group consisting of:
(a) inactivated M. vaccae cells;
(b) delipidated and deglycolipidated M. vaccae cells;
(c) delipidated and deglycolipidated M. vaccae cells that have been treated by alkaline hydrolysis;
(d) delipidated and deglycolipidated M. vaccae cells that have been treated by acid hydrolysis;
(e) delipidated and deglycolipidated M. vaccae cells that have been treated with periodic acid;
(f) delipidated and deglycolipidated M. vaccae cells that have been treated by alkaline hydrolysis and by acid hydrolysis;
(g) delipidated and deglycolipidated M. vaccae cells that have been treated by alkaline hydrolysis and treated with periodic acid;
(h) delipidated and deglycolipidated M. vaccae cells that have been treated with Proteinase K; and
(i) delipidated and deglycolipidated M. vaccae cells that have been treated by hydrofluoric acid hydrolysis.

4. A method for stimulating infectious tolerance to an antigen in a subject, comprising administering to the subject a composition comprising at least one component selected from the group consisting of:
(a) inactivated M. vaccae cells;
(b) delipidated and deglycolipidated M. vaccae cells;
(c) delipidated and deglycolipidated M. vaccae cells that have been treated by alkaline hydrolysis;
(d) delipidated and deglycolipidated M. vaccae cells that have been treated by acid hydrolysis;
(e) delipidated and deglycolipidated M. vaccae cells that have been treated with periodic acid;
(f) delipidated and deglycolipidated M. vaccae cells that have been treated by alkaline hydrolysis and by acid hydrolysis;
(g) delipidated and deglycolipidated M. vaccae cells that have been treated by alkaline hydrolysis and treated with periodic acid;
(h) delipidated and deglycolipidated M. vaccae cells that have been treated with Proteinase K; and
(i) delipidated and deglycolipidated M. vaccae cells that have been treated by hydrofluoric acid hydrolysis.

5. A method for treating a disorder characterized by the presence of an abnormal immune response in a subject, the method comprising administering to the subject a composition comprising at least one component selected from the group consisting of:
(a) inactivated M. vaccae cells;
(b) delipidated and deglycolipidated M. vaccae cells;
(c) delipidated and deglycolipidated M. vaccae cells that have been treated by alkaline hydrolysis;
(d) delipidated and deglycolipidated M. vaccae cells that have been treated by acid hydrolysis;
(e) delipidated and deglycolipidated M. vaccae cells that have been treated with periodic acid;
(f) delipidated and deglycolipidated M. vaccae cells that have been treated by alkaline hydrolysis and by acid hydrolysis;
(g) delipidated and deglycolipidated M. vaccae cells that have been treated by alkaline hydrolysis and treated with periodic acid;
(h) delipidated and deglycolipidated M. vaccae cells that have been treated with Proteinase K; and
(i) delipidated and deglycolipidated M. vaccae cells that have been treated by hydrofluoric acid hydrolysis.

6. A method for modulating Notch signaling in a population of cells, comprising contacting the cells with a composition comprising at least one component selected from the group consisting of:
(a) inactivated M. vaccae cells;
(b) delipidated and deglycolipidated M. vaccae cells;
(c) delipidated and deglycolipidated M. vaccae cells that have been treated by alkaline hydrolysis;
(d) delipidated and deglycolipidated M. vaccae cells that have been treated by acid hydrolysis;
(e) delipidated and deglycolipidated M. vaccae cells that have been treated with periodic acid;
(f) delipidated and deglycolipidated M. vaccae cells that have been treated by alkaline hydrolysis and by acid hydrolysis;
(g) delipidated and deglycolipidated M. vaccae cells that have been treated by alkaline hydrolysis and treated with periodic acid;
(h) delipidated and deglycolipidated M. vaccae cells that have been treated with Proteinase K; and
(i) delipidated and deglycolipidated M. vaccae cells that have been treated by hydrofluoric acid hydrolysis.

7. A method for modulating Notch signaling in a population of cells, comprising contacting the cells with a composition comprising an isolated polypeptide, wherein the polypeptide comprises a sequence selected from the group consisting of:
(a) SEQ ID NO: 27-52;
(b) sequences encoded by a sequence of SEQ ID NO: 1-26;
(c) sequence having at least 75% identity to a sequence of SEQ ID NO: 27-52; and
(d) sequences having at least 90% identity to a sequence of SEQ ID NO: 27-52.

8. A method for modulating Notch signaling in a population of cells, comprising contacting the cells with a composition comprising a component selected from the group consisting of:
(a) delipidated and deglycolipidated M. smegmatis cells; and
(b) delipidated and deglycolipidated M. tuberculosis cells.

9. A method for modulating expression of a Notch signaling gene in a population of cells, comprising contacting the cells with a composition comprising a component selected from the group consisting of:
(a) inactivated M. vaccae cells;
(b) delipidated and deglycolipidated M. vaccae cells;
(c) delipidated and deglycolipidated M. vaccae cells that have been treated by alkaline hydrolysis;
(d) delipidated and deglycolipidated M. vaccae cells that have been treated by acid hydrolysis;
(e) delipidated and deglycolipidated M. vaccae cells that have been treated with periodic acid;
(f) delipidated and deglycolipidated M. vaccae cells that have been treated by alkaline hydrolysis and by acid hydrolysis;
(g) delipidated and deglycolipidated M. vaccae cells that have been treated by alkaline hydrolysis and treated with periodic acid;
(h) delipidated and deglycolipidated M. vaccae cells that have been treated with Proteinase K; and
(i) delipidated and deglycolipidated M. vaccae cells that have been treated by hydrofluoric acid hydrolysis.

10. The method of claim 9, wherein the Notch signaling molecule is selected from the group consisting of: Notch1, Notch2, Notch3, Notch4, Deltex, Jagged-1, Jagged-2, Delta-like 1, Delta-like 3, HES-1, HERP1, HERP2, Lunatic Fringe, Manic Fringe, Radical Fringe, Numb, MAML1 and RBP-Jkappa.

11. A method for modulating expression of a Toll-like receptor gene in a population of cells, comprising contacting the cells with a composition comprising a component selected from the group consisting of:
(a) inactivated M. vaccae cells;
(b) delipidated and deglycolipidated M. vaccae cells;
(c) delipidated and deglycolipidated M. vaccae cells that have been treated by alkaline hydrolysis;
(d) delipidated and deglycolipidated M. vaccae cells that have been treated by acid hydrolysis;
(e) delipidated and deglycolipidated M. vaccae cells that have been treated with periodic acid;
(f) delipidated and deglycolipidated M. vaccae cells that have been treated by alkaline hydrolysis and by acid hydrolysis;
(g) delipidated and deglycolipidated M. vaccae cells that have been treated by alkaline hydrolysis and treated with periodic acid;
(h) delipidated and deglycolipidated M. vaccae cells that have been treated with Proteinase K; and
(i) delipidated and deglycolipidated M. vaccae cells that have been treated by hydrofluoric acid hydrolysis.

12. A method for modulating Notch signaling in a population of cells, comprising contacting the cells with a composition comprising peptidoglycan.

13. A method for modulating Toll-like receptor signaling in a population of cells, comprising contacting the cells with a composition comprising peptidoglycan.

14. A method for modulating Toll-like receptor signaling in a population of cells, comprising contacting the cells with a composition comprising a component selected from the group consisting of:
(a) inactivated M. vaccae cells;
(b) delipidated and deglycolipidated M. vaccae cells;
(c) delipidated and deglycolipidated M. vaccae cells that have been treated by alkaline hydrolysis;
(d) delipidated and deglycolipidated M. vaccae cells that have been treated by acid hydrolysis;
(e) delipidated and deglycolipidated M. vaccae cells that have been treated with periodic acid;
(f) delipidated and deglycolipidated M. vaccae cells that have been treated by alkaline hydrolysis and by acid hydrolysis;
(g) delipidated and deglycolipidated M. vaccae cells that have been treated by alkaline hydrolysis and treated with periodic acid;
(h) delipidated and deglycolipidated M. vaccae cells that have been treated with Proteinase K; and
(i) delipidated and deglycolipidated M. vaccae cells that have been treated by hydrofluoric acid hydrolysis.


SEQUENCE LISTING (52 sequences)

SEQ ID NO: 1 - DNA, 683 bp, Mycobacterium vaccae
gcccgccaactaaaaccgccgatcatccactgcaggaaggaatctcacgatcatgaacatcagcatgaaaactcttgccggagcgggtttcgcgatgaccgccgccgtcggtctgtcgctgggtaccgcaggcagcgccgcagccgcgccggtcggaccggggtgtgcggcctacgtgcaacaggtgccggacgggccgggatcggtgcagggcatggcgagctcgccggtggccaccgcggcggccgacaacccgctgctcaccacgctctcgcaggcgatctcgggtcagctcaacccgaacgtcaatctcgtcgacacgttcaacggcggccagttcaccgtgttcgcgccgaccaatgacgccttcgccaagatcgatccggccacgctggagaccctcaagaccgattccgacctgctgaccaagatcctcacctaccacgtcgtgcccggccaggccgcgcccgatcaggtggtcggcgagcatgtgacggtggagggggcgccggtcacggtgtccgggatggccgaccagctcaaggtcaacgacgcgtcggtggtgtgcggtggggtgcagaccgccaacgcgacggtgtatctgatcgacaccgtgctgatgccgccggcagcgtagccgggcggcaccacagaagagggtcccccgcacccggcctcccccg

SEQ ID NO: 2 - DNA, 808 bp, Mycobacterium vaccae
  misc_feature (1)...(808): n = A,T,C or G
ccaagtgtgacgcgngtgtgacggtagacgttccgaccaatccaacgacgccgcagctgggaatcacccgtgtgccaattcagtgcgggcaacggtgtccgtccacgaagggattcaggaaatgatgacaactcgccggaagtcagccgcagtggcgggaatcgctgcggtggccatcctcggtgcggccgcatgttcgagtgaggacggtgggagcacggcctcgtcggccagcagcacggcctcctccgcgatggagtccgcgaccgacgagatgaccacgtcgtcggcggccccttcggccgaccctgcggccaacctgatcggctccggctgcgcggcctacgccgagcaggtccccgaaggtcccgggtcggtggccgggatggcagccgatccggtgacggtggcggcgtcgaacaacccgatgctgcagacgctgtcccaggcgctgtccggccagctcaatccgcaggtcaatctcgtcgacaccctcgacggcggtgagttcaccgtgttcgcgccgaccgacgacgcgttcgccaagatcgatccggccacgctggagaccctcaagacggactccgacatgctgaccaacatcctgacctaccacgtcgtgcccggccaggccgcgcccgatcaggtggtcggcgagcatgtgacggtggagggggcgccggtcacggtgtccgggatggccgaccagctcaaggtcaacgacgcgtcggtggtgtgcggtggggtgcagaccgccaacgcgacggtgtatctgatcgacaccgtgctgatgccgccggcagcgtag

SEQ ID NO: 3 - DNA, 1211 bp, Mycobacterium vaccae
ggtaccggaagctggaggattgacggtatgagacttcttgacaggattcgtgggccttgggcacgccgtttcggcgtcgtggctgtcgcgacagcgatgatgcctgctttggtgggcctggctggagggtcggcgaccgccggagcattctcccggccaggtctgccggtggagtacctgatggtgccttcgccgtcgatggggcgcgacatcaagatccagttccagagcggtggcgagaactcgccggctctctacctgctcgacggcctgcgtgcgcaggaggacttcaacggctgggacatcaacactcaggctttcgagtggttcctcgacagcggcatctccgtggtgatgccggtcggtggccagtccagcttctacaccgactggtacgcccccgcccgtaacaagggcccgaccgtgacctacaagtgggagaccttcctgacccaggagctcccgggctggctgcaggccaaccgcgcggtcaagccgaccggcagcggccctgtcggtctgtcgatggcgggttcggccgcgctgaacctggcgacctggcacccggagcagttcatctacgcgggctcgatgtccggcttcctgaacccctccgagggctggtggccgttcctgatcaacatctcgatgggtgacgccggcggcttcaaggccgacgacatgtggggcaagaccgaggggatcccaacagcggttggacagcgcaacgatccgatgctgaacatcccgaccctggtcgccaacaacacccgtatctgggtctactgcggtaacggccagcccaccgagctcggcggcggcgacctgcccgccacgttcctcgaaggtctgaccatccgcaccaacgagaccttccgcgacaactacatcgccgcgggtggccacaacggtgtgttcaacttcccggccaacggcacgcacaactgggcgtactggggtcgcgagctgcaggcgatgaagcctgacctgcaggcgcaccttctctgacggttgcacgaaacgaagcccccggccgattgcggccgagggtttcgtcgtccggggctactgtggccgacataaccgaaatcaacgcgatggtggctcatcaggaacgccgagggggtcattgcgctacgacacgaggtgggcgagcaatccttcctgcccgacggagaggtcaacatccacgtcgagtactccagcgtgaa

SEQ ID NO: 4 - DNA, 485 bp, Mycobacterium vaccae
agcggctgggacatcaacaccgccgccttcgagtggtacgtcgactcgggtctcgcggtgatcatgcccgtcggcgggcagtccagcttctacagcgactggtacagcccggcctgcggtaaggccggctgccagacctacaagtgggagacgttcctgacccaggagctgccggcctacctcgccgccaacaagggggtcgacccgaaccgcaacgcggccgtcggtctgtccatggccggttcggcggcgctgacgctggcgatctaccacccgcagcagttccagtacgccgggtcgctgtcgggctacctgaacccgtccgaggggtggtggccgatgctgatcaacatctcgatgggtgacgcgggcggctacaaggccaacgacatgtggggtcgcaccgaggacccgagcagcgcctggaagcgcaacgacccgatggtcaacatcggcaagctggtcgccaacaacacccccctctc

SEQ ID NO: 5 - DNA, 1052 bp, Mycobacterium vaccae
gttgatgagaaaggtgggttgtttgccgttatgaagttcacagagaagtggcggggctccgcaaaggcggcgatgcaccgggtgggcgttgccgatatggccgccgttgcgctgcccggactgatcggcttcgccgggggttcggcaacggccggggcattctcccggcccggtcttcctgtcgagtacctcgacgtgttctcgccgtcgatgggccgcgacatccgggtccagttccagggtggcggtactcatgcggtctacctgctcgacggtctgcgtgcccaggacgactacaacggctgggacatcaacacccctgcgttcgagtggttctacgagtccggcttgtcgacgatcatgccggtcggcggacagtccagcttctacagcgactggtaccagccgtctcggggcaacgggcagaactacacctacaagtgggagacgttcctgacccaggagctgccgacgtggctggaggccaaccgcggagtgtcgcgcaccggcaacgcgttcgtcggcctgtcgatggcgggcagcgcggcgctgacctacgcgatccatcacccgcagcagttcatctacgcctcgtcgctgtcaggcttcctgaacccgtccgagggctggtggccgatgctgatcgggctggcgatgaacgacgcaggcggcttcaacgccgagagcatgtggggcccgtcctcggacccggcgtggaagcgcaacgacccgatggtcaacatcaaccagctggtggccaacaacacccggatctggatctactgcggcaccggcaccccgtcggagctggacaccgggaccccgggccagaacctgatggccgcgcagttcctcgaaggattcacgttgcggaccaacatcgccttccgtgacaactacatcgcagccggcggcaccaacggtgtcttcaacttcccggcctcgggcacccacagctgggggtactgggggcagcagctgcagcagatgaagcccgacatccagcgggttctgggagctcaggccaccgcctagccacccaccccacaccc

SEQ ID NO: 6 - DNA, 480 bp, Mycobacterium vaccae
aacggctgggacatcaacacccctgcgttcgagtggttctacgagtccggcttgtcgacgatcatgccggtcggcggacagtccagcttctacagcgactggtaccagccgtctcggggcaacgggcagaactacacctacaagtgggagacgttcctgacccaggagctgccgacgtggctggaggccaaccgcggagtgtcgcgcaccggcaacgcgttcgtcggcctgtcgatggcgggcagcgcggcgctgacctacgcgatccatcacccgcagcagttcatctacgcctcgtcgctgtcaggcttcctgaacccgtccgagggctggtggccgatgctgatcgggctggcgatgaacgacgcaggcggcttcaacgccgagagcatgtggggcccgtcctcggacccggcgtggaagcgcaacgacccgatggtcaacatcaaccagctggtggccaacaacacccggatctgg

SEQ ID NO: 7 - DNA, 795 bp, Mycobacterium vaccae
ctgccgcgggtttgccatctcttgggtcctgggtcgggaggccatgttctgggtaacgatccggtaccgtccggcgatgtgaccaacatgcgaacagcgacaacgaagctaggagcggcgctcggcgcagcagcattggtggccgccacggggatggtcagcgcggcgacggcgaacgcccaggaagggcaccaggtccgttacacgctcacctcggccggcgcttacgagttcgacctgttctatctgacgacgcagccgccgagcatgcaggcgttcaacgccgacgcgtatgcgttcgccaagcgggagaaggtcagcctcgccccgggtgtgccgtgggtcttcgaaaccacgatggccgacccgaactgggcgatccttcaggtcagcagcaccacccgcggtgggcaggccgccccgaacgcgcactgcgacatcgccgtcgatggccaggaggtgctcagccagcacgacgacccctacaacgtgcggtgccagctcggtcagtggtgagtcacctcgccgagagtccggccagcgccggcggcagcggctcgcggtgcagcaccccgaggcgctgggtcgcgcgggtcagcgcgacgtaaagatcgctggccccgcgcggcccctcggcgaggatctgctccgggtagaccaccagcacggcgtctaactccagacccttggtctgcgtgggtgccaccgcgcccgggacaccgggcgggccgatcaccacgctggtgccctcccggtccgcctccgcacgcacgaaatcgtcgatggcaccggcga

SEQ ID NO: 8 - DNA, 1125 bp, Mycobacterium vaccae
  misc_feature (1)...(1125): n = A,T,C or G
atgcaggtgcggcgtgttctgggcagtgtcggtgcagcagtcgcggtttcggccgcgttatggcagacgggggtttcgataccgaccgcctcagcggatccgtgtccggacatcgaggtgatcttcgcgcgcgggaccggtgcggaacccggcctcgggtgggtcggtgatgcgttcgtcaacgcgctgcggcccaaggtcggtgagcagtcggtgggcacctacgcggtgaactacccggcaggattcggacttcgacaaatcggcgcccatgggcgcggccgacgcatcggggcgggtgcagtggatggccgacaactgcccggacaccaagcttgtcctgggcggcatgtcgcanggcgccggcgtcatcgacctgatcaccgtcgatccgcgaccgctgggccggttcacccccaccccgatgccgccccgcgtcgccgaccacgtggccgccgttgtggtcttcggaaatccgttgcgcgacatccgtggtggcggtccgctgccgcagatgagcggcacctacgggccgaagtcgatcgatctgtgtgcgctcgacgatccgttctgctcgcccggcttcaacctgccggcccacttcgcctacgccgacaacggcatggtggaggaagccgcgaacttcgcccgcctggaaccgggccagagcgtcgagctgcccgaggcgccctacctgcacctgttcgtcccgcggggcgaggtaacgctggaggacgccggaccgctgcgcgaaggcgacgcagtgcgtttcaccgcatcgggcggccagcgggtgaccgccaccgcgcccgcggagatcctcgtctgggagatgcatgcgggactcggtgcggcataagcgaataggagtcctgctggccggcgcagcactgctcgccggatgcacatccgaacctggacccgggccgtcggcggcaccggccccgacgagcacaaccgagagcgcacccggtcccggactcgtcccggtgaccgtcgcggtcgacgaacctctggccgacgcgccgttcgaccagccccgggaggccctggtgccgcagggttggacgctgtcggtgtgggcgcggaccgcccggccgcggctggccgcgtgggccccggacg

SEQ ID NO: 9 - DNA, 650 bp, Mycobacterium vaccae
gacacaccagcaccactgttaacctcgctagatcagtcggccgaacggaaggacagccgtgaccctgaaaaccctagtcaccagcatgaccgctggggcagcagcagccgcaacactcggcgctgccgccgtgggtgtgacctcgattgccgtcggtgcgggtgtcgccggcgcgtcgcccgcggtgctgaacgcaccgctgctttccgcccctgcccccgatctgcagggaccgctggtctccaccttgagcgcgctgtcgggcccgggctccttcgccggcgccaaggccacctacgtccagggcggtctcggccgcatcgaggcccgggtggccgacagcggatacagcaacgccgcggccaagggctacttcccgctgagcttcaccgtcgccggcatcgaccagaacggtccgatcgtgaccgccaacgtcaccgcggcggccccgacgggcgccgtggccacccagccgctgacgttcatcgccgggccgagcccgaccggatggcagctgtccaagcagtccgcactggccctgatgtccgcggtgggtgatctcccgcacgattctggtccgcagcgccgtcacatgtgtggcggcgctcgggctgggtgggtgcctgggcggctgcgcgcaagatgaacat

SEQ ID NO: 10 - DNA, 501 bp, Mycobacterium vaccae
  misc_feature (1)...(501): n = A,T,C or G
atgccggtgcgacgtgcgcgcagtgcgcttgcgtccgtgaccttcgtcgcggccgcgtgcgtgggcgctgagggcaccgcactggcggcgacgccggactggagcgggcgctacacggtggtgacgttcgcctccgacaaactcggcacgagtgtggccgcccgccagccagaacccgacttcagcggtcagtacaccttcagcacgtcctgtgtgggcacctgcgtggccaccgcgtccgacggcccggcgccgtcgaacccgacgattccgcagcccgcgcgctacacctgggacggcaggcagtgggtgttcaactacaactggcagtgggagtgcttccgcggcgccgacgtcccgcgcgagtacgccgccgcgcgttcgctggtgttctacgccccgaccgccgacgggtcgatgttcggcacctggcgcaccganatcctgganggcctctgcaagggcaccgtgatcatgccggtcgcggcctatccggcgtag

SEQ ID NO: 11 - DNA, 554 bp, Mycobacterium vaccae
gatgtcacgcccggagaatgtaacgttcgaccggagaacgccgtcggcacaacgagttacgtttgagcacttcagatctcggttaccttggatttcaggcgggggaagcagtaaccgatccaagattcgaaggacccaaacaacatgaaattcactggaatgaccgtgcgcgcaagccgcgcgccctggccggcgtcggggcggcatgtctgttcggcggcgtggccgcggcaaccgtggcggcacagatggcgggcgcccagccggccgagtgcaacgccagctcactcaccggcaccgtcagctcggtgaccggtcaggcgcgtcagtacctagacacccacccgggcgccaaccaggccgtcaccgcggcgatgaaccagccgcggcccgaggccgaggcgaacctgcggggctacttcaccgccaacccggcggagtactacgacctgcggggcatcctcgccccgatcggtgacgcgcagcgcaactgcaacatcaccgtgctgccggtagagctgcagacggcctacgacacgttcatggccggctga

SEQ ID NO: 12 - DNA, 1518 bp, Mycobacterium vaccae
cactcgccatgggtgttacaataccccaccagttcctcgaagtaaacgaacagaaccgtgacatccagctgagaaaatattcacagcgacgaagcccggccgatgcctgatggggtccggcatcagtacagcgcgctttcctgcgcggattctattgtcgagtccggggtgtgacgaaggaatccattgtcgaaatgtaaattcgttgcggaatcacttgcataggtccgtcagatccgcgaaggtttaccccacagccacgacggctgtccccgaggaggacctgccctgaccggcacacacatcaccgctgcagaacctgcagaacagacggcggattccgcggcaccgcccaagggcgcgccggtgatcgagatcgaccatgtcacgaagcgcttcggcgactacctggccgtcgcggacgcagacttctccatcgcgcccggggagttcttctccatgctcggcccgtccgggtgtgggaagacgaccacgttgcgcatgatcgcgggattcgagaccccgactgaaggggcgatccgcctcgaaggcgccgacgtgtcgaggaccccacccaacaagcgcaacgtcaacacggtgttccagcactacgcgctgttcccgcacatgacggtctgggacaacgtcgcgtacggcccgcgcagcaagaaactcggcaaaggcgaggtccgcaagcgcgtcgacgagctgctggagatcgtccggctgaccgaatttgccgagcgcaggcccgcccagctgtccggcgggcagcagcagcgggtggcgttggcccgggcactggtgaactaccccagcgcgctgctgctcgatgaaccgctcggagcgctcgacctgaagctgcgccacgtcatgcagttcgagctcaagcgcatccagcgggaggtcgggatcacgttcatctacgtgacccacgaccaggaagaggcgctcacgatgagtgaccgcatcgcggtgatgaacgccggcaacgtcgaacagatcggcagcccgaccgagatctacgaccgtcccgcgacggtgttcgtcgccagcttcatcggacaggccaacctctgggcgggccggtgcaccggccgctccaaccgcgattacgtcgagatcgacgttctcggctcgacgctgaaggcacgcccgggcgagaccacgatcgagcccggcgggcacgccaccctgatggtgcgtccggaacgcatccgggtcaccccgggctcccaggacgcgccgaccggtgacgtcgcctgcgtgcgtgccaccgtcaccgacctgaccttccaaggtccggtggtgcggctctcgctggccgctccggacgactcgaccgtgatcgcccacgtcggccccgagcaggatctgccgctgctgcgccccggcgacgacgtgtacgtcagctgggcaccggaagcctccctggtgcttcccggcgacgacatccccaccaccgaggacctcgaagagatgctcgacgactcctgagtcacgcttcccgattgccga

SEQ ID NO: 13 - DNA, 1111 bp, Mycobacterium vaccae
gtccgacagtgggacctcgagcaccacgtcacaggacagcggccccgccagcggcgccctgcgcgtctccaactggccgctctatatggccgacggtttcatcgcagcgttccagaccgcctcgggcatcacggtcgactacaaagaagacttcaacgacaacgagcagtggttcgccaaggtcaaggagccgttgtcgcgcaagcaggacataggcgccgacctggtgatccccaccgagttcatggccgcgcgcgtcaagggcctgggatggctcaatgagatcagcgaagccggcgtgcccaatcgcaagaatctgcgtcaggacctgttggactcgagcatcgacgagggccgcaagttcaccgcgccgtacatgaccggcatggtcggtctcgcctacaacaaggcagccaccggacgcgatatccgcaccatcgacgacctctgggatcccgcgttcaagggccgcgtcagtctgttctccgacgtccaggacggcctcggcatgatcatgctctcgcagggcaactcgccggagaatccgaccaccgagtccattcagcaggcggtcgatctggtccgcgaacagaacgacagggggtcagatccgtcgcttcaccggcaacgactacgccgacgacctggccgcagaaacatcgccatcgcgcaggcgtactccggtgacgtcgtgcagctgcaggcggacaaccccgatctgcagttcatcgttcccgaatccggcggcgactggttcgtcgacacgatggtgatcccgtacaccacgcagaaccagaaggccgccgaggcgtggatcgactacatctacgaccgagccaactacgccaagctggtcgcgttcacccagttcgtgcccgcactctcggacatgaccgacgaactcgccaaggtcgatcctgcatcggcggagaacccgctgatcaacccgtcggccgaggtgcaggcgaacctgaagtcgtgggcggcactgaccgacgagcagacgcaggagttcaacactgcgtacgccgccgtcaccggcggctgacgcggtggtagtgccgatgcgaggggcataaatggccctgcggacgcgaggagcataaatggc

SEQ ID NO: 14 - DNA, 1626 bp, Mycobacterium vaccae
atggccaagacaattgcgtatgacgaagaggcccgccgtggcctcgagcggggcctcaacgccctcgcagacgccgtaaaggtgacgttgggcccgaagggtcgcaacgtcgtgctggagaagaagtggggcgcccccacgatcaccaacgatggtgtgtccatcgccaaggagatcgagctggaggacccgtacgagaagatcggcgctgagctggtcaaagaggtcgccaagaagaccgacgacgtcgcgggcgacggcaccaccaccgccaccgtgctcgctcaggctctggttcgcgaaggcctgcgcaacgtcgcagccggcgccaacccgctcggcctcaagcgtggcatcgagaaggctgtcgaggctgtcacccagtcgctgctgaagtcggccaaggaggtcgagaccaaggagcagatttctgccaccgcggcgatttccgccggcgacacccagatcggcgagctcatcgccgaggccatggacaaggtcggcaacgagggtgtcatcaccgtcgaggagtcgaacaccttcggcctgcagctcgagctcaccgagggtatgcgcttcgacaagggctacatctcgggttacttcgtgaccgacgccgagcgccaggaagccgtcctggaggatccctacatcctgctggtcagctccaaggtgtcgaccgtcaaggatctgctcccgctgctggagaaggtcatccaggccggcaagccgctgctgatcatcgccgaggacgtcgagggcgaggccctgtccacgctggtggtcaacaagatccgcggcaccttcaagtccgtcgccgtcaaggctccgggcttcggtgaccgccgcaaggcgatgctgcaggacatggccatcctcaccggtggtcaggtcgtcagcgaaagagtcgggctgtccctggagaccgccgacgtctcgctgctgggccaggcccgcaaggtcgtcgtcaccaaggacgagaccaccatcgtcgagggctcgggcgattccgatgccatcgccggccgggtggctcagatccgcgccgagatcgagaacagcgactccgactacgaccgcgagaagctgcaggagcgcctggccaagctggccggcggtgttgcggtgatcaaggccggagctgccaccgaggtggagctcaaggagcgcaagcaccgcatcgaggacgccgtccgcaacgcgaaggctgccgtcgaagagggcatcgtcgccggtggcggcgtggctctgctgcagtcggctcctgcgctggacgacctcggcctgacgggcgacgaggccaccggtgccaacatcgtccgcgtggcgctgtcggctccgctcaagcagatcgccttcaacggcggcctggagcccggcgtcgttgccgagaaggtgtccaacctgcccgcgggtcacggcctcaacgccgcgaccggtgagtacgaggacctgctcaaggccggcgtcgccgacccggtgaaggtcacccgctcggcgctgcagaacgcggcgtccatcgcggctctgttcctcaccaccgaggccgtcgtcgccgacaagccggagaaggcgtccgcacccgcgggcgacccgaccggtggcatgggcggtatggacttctaa

SEQ ID NO: 15 - DNA, 647 bp, Mycobacterium vaccae
atggccaagacaattgcgtatgacgaagaggcccgccgtggcctcgagcggggcctcaacgccctcgcagacgccgtaaaggtgacgttgggcccgaagggtcgcaacgtcgtgctggagaagaagtggggcgcccccacgatcaccaacgatggtgtgtccatcgccaaggagatcgagctggaggacccgtacgagaagatcggcgctgagctggtcaaagaggtcgccaagaagaccgacgacgtcgcgggcgacggcaccaccaccgccaccgtgctcgctcaggctctggttcgcgaaggcctgcgcaacgtcgcagccggcgccaacccgctcggcctcaagcgtggcatcgagaaggctgtcgaggctgtcacccagtcgctgctgaagtcggccaaggaggtcgagaccaaggagcagatttctgccaccgcggcgatttccgccggcgacacccagatcggcgagctcatcgccgaggccatggacaaggtcggcaacgagggtgtcatcaccgtcgaggagtcgaacaccttcggcctgcagctcgagctcaccgagggtatgcgcttcgacaagggctacatctcgggttacttcgtgaccgacgccgagcgccaggaagccgtcctggaggatcc

SEQ ID NO: 16 - DNA, 985 bp, Mycobacterium vaccae
ggatccctacatcctgctggtcagctccaaggtgtcgaccgtcaaggatctgctcccgctgctggagaaggtcatccaggccggcaagccgctgctgatcatcgccgaggacgtcgagggcgaggccctgtccacgctggtggtcaacaagatccgcggcaccttcaagtccgtcgccgtcaaggctccgggcttcggtgaccgccgcaaggcgatgctgcaggacatggccatcctcaccggtggtcaggtcgtcagcgaaagagtcgggctgtccctggagaccgccgacgtctcgctgctgggccaggcccgcaaggtcgtcgtcaccaaggacgagaccaccatcgtcgagggctcgggcgattccgatgccatcgccggccgggtggctcagatccgcgccgagatcgagaacagcgactccgactacgaccgcgagaagctgcaggagcgcctggccaagctggccggcggtgttgcggtgatcaaggccggagctgccaccgaggtggagctcaaggagcgcaagcaccgcatcgaggacgccgtccgcaacgcgaaggctgccgtcgaagagggcatcgtcgccggtggcggcgtggctctgctgcagtcggctcctgcgctggacgacctcggcctgacgggcgacgaggccaccggtgccaacatcgtccgcgtggcgctgtcggctccgctcaagcagatcgccttcaacggcggcctggagcccggcgtcgttgccgagaaggtgtccaacctgcccgcgggtcacggcctcaacgccgcgaccggtgagtacgaggacctgctcaaggccggcgtcgccgacccggtgaaggtcacccgctcggcgctgcagaacgcggcgtccatcgcggctctgttcctcaccaccgaggccgtcgtcgccgacaagccggagaaggcgtccgcacccgcgggcgacccgaccggtggcatgggcggtatggacttctaa

SEQ ID NO: 17 - DNA, 743 bp, Mycobacterium vaccae
ggatccgcggcaccggctggtgacgaccaagtacaacccggcccgcacctggacggccgagaactccgtcggcatcggcggcgcgtacctgtgcatctacgggatggagggccccggcggctatcagttcgtcggccgcaccacccaggtgtggagtcgttaccgccacacggcgccgttcgaacccggaagtccctggctgctgcggtttttcgaccgaatttcgtggtatccggtgtcggccgaggagctgctggaattgcgagccgacatggccgcaggccggggctcggtcgacatcaccgacggcgtgttctccctcgccgagcacgaacggttcctggccgacaacgccgacgacatcgccgcgttccgttcccggcaggcggccgcgttctccgccgagcggaccgcgtgggcggccgccggcgagttcgaccgcgccgagaaagccgcgtcgaaggccaccgacgccgataccggggacctggtgctctacgacggtgacgagcgggtcgacgctccgttcgcgtcgagcgtgtggaaggtcgacgtcgccgtcggtgaccgggtggtggccggacagccgttgctggcgctggaggcgatgaagatggagaccgtgctgcgcgccccggccgacggggtggtcacccagatcctggtctccgctgggcatctcgtcgatcccggcaccccactggtcgtggtcggcaccggagtgcgcgcatgagcgccgtcga

SEQ ID NO: 18 - DNA, 1164 bp, Mycobacterium vaccae
ggtggcgcgcatcgagaagcgcccgccccggttcacgggcgcctgatcatggtgcgggcggcgctgcgctacggcttcgggacggcctcactgctggccggcgggttcgtgctgcgcgccctgcagggcacgcctgccgccctcggcgcgactccgggcgaggtcgcgccggtggcgcgccgctcgccgaactaccgcgacggcaagttcgtcaacctggagcccccgtcgggcatcacgatggatcgcgacctgcagcggatgctgttgcgcgatctggccaacgccgcatcccagggcaagccgcccggaccgatcccgctggccgagccgccgaagggggatcccactcccgcgccggcggcggccagctggtacggccattccagcgtgctgatcgaggtcgacggctaccgcgtgctggccgacccggtgtggagcaacagatgttcgccctcacgggcggtcggaccgcagcgcatgcacgacgtcccggtgccgctggaggcgcttcccgccgtggacgcggtggtgatcagccacgaccactacgaccacctcgacatcgacaccatcgtcgcgttggcgcacacccagcgggccccgttcgtggtgccgttgggcatcggcgcacacctgcgcaagtggggcgtccccgaggcgcggatcgtcgagttggactggcacgaagcccaccgcatagacgacctgacgctggtctgcacccccgcccggcacttctccggacggttgttctcccgcgactcgacgctgtgggcgtcgtgggtggtcaccggctcgtcgcacaaggcgttcttcggtggcgacaccggatacacgaagagcttcgccgagatcggcgacgagtacggtccgttcgatctgaccctgctgccgatcggggcctaccatcccgcgttcgccgacatccacatgaaccccgaggaggcggtgcgcgcccatctggacctgaccgaggtggacaacagcctgatggtgcccatccactgggcgacattccgcctcgccccgcatccgtggtccgagcccgccgaacgcctgctgaccgctgccgacgccgagcgggtacgcctgaccgtgccgattcccggtcagcgggtggacccggagtcgacgttcgacccgtggtggcggttctgaacc

SEQ ID NO: 19 - DNA, 1012 bp, Mycobacterium vaccae
atgaaggcaaatcattcgggatgctacaaatccgccggcccgatatggtcgcatccatcgccgctttgttcgcccgcactggcaccatctcatgcaggtctggacaatgagctgagcctgggcatccacggccagggcccggaacgactgaccattcagcagtgggacaccttcctcaacggcgtcttcccgttggaccgcaaccggttgacccgggagtggttccactcgggcaaggcgacctacgtcgtggccggtgaaggtgccgacgagttcgagggcacgctggagctgggctaccaggtgggctttccgtggtcgctgggcgtgggcatcaacttcagctacaccaccccgaacatcacgtacgacggttacggcctcaacttcgccgacccgctgctgggcttcggtgattccatcgtgaccccgccgctgttcccgggtgtctcgatcacggcggacctgggcaacggccccggcatccaggaggtcgcgaccttctccgtggacgtggccggccccggtggttccgtggtggtgtccaacgcgcacggcacggtcaccggtgctgccggtggtgtgctgctgcgtccgttcgcccgcctgatctcgtcgaccggcgacagcgtcaccacctacggcgcaccctgctgaaacatgaactgaccacatcacgatggaggccccccggcgtcaaccggggcccgcttcacgctggtcgggaggcgcccgaggttcgatcgaagtggccgactgcggcaaacgcctgcgcgcgcgattcttcgagtctgacgcagggtctggtggtagtcgaatgtcatcctgtgactccacctcatcgcccgagacgcgacggccggggttccggtgtgtgggcgccggccttgggcacgtacgggggcgaccgacgtcgtgatgtgacgagcgtcgcagtgtttgccggcaacccggacggcccggccgagtccccgcatccgtccagcgaacccgggggatccaaagaattcag

SEQ ID NO: 20 - DNA, 898 bp, Mycobacterium vaccae
gagcaaccgttccggctcggcgactggatcaccgtccccaccgcggcgggccggccgtccgcccacggccgcgtggtggaagtcaactggcgtgcaacacatatcgacaccggcggcaacctgctggtaatgcccaacgccgaactcgccggcgcgtcgttcaccaattacagccggcccgtgggagagcaccggctgaccgtcgtcaccaccttcaacgccgcggacacccccgatgatgtctgcgagatgctgtcgtcggtcgcggcgtcgctgcccgaactgcgcaccgacggacagatcgccacgctctatctcggtgcggccgaatacgagaagtcgatcccgttgcacacacccgcggtggacgactcggtcaggagcacgtacctgcgatgggtctggtacgccgcgcgccggcaggaacttcgcctaacggcgtcgccgacgattcgacacgccggaacggatcgcctcggccatgcgggctgtggcgtccacactgcgcttggcagacgacgaacagcaggagatcgccgacgtggtgcgtctggtccgttacggcaacggggaacgcctccagcagccgggtcaggtaccgaccgggatgaggttcatcgtagacggcagggtgagtctgtccgtgatcgatcaggacggcgacgtgatcccggcgcgggtgctcgagcgtggcgacttcctggggcagaccacgctgacgcgggaaccggtactggcgaccgcgcacgcgctggaggaagtcaccgtgctggagatggcccgtgacgagatcgagcgcctggtgcaccgaaagccgatcctgctgcacgtgatcggggccgtgatcgccgaccggcgcgcgcacgaacttcggttgatggcggactcgcaggactga

SEQ ID NO: 21 - DNA, 2013 bp, Mycobacterium vaccae
ggctatcagtccggacggtcctcgctgcgcgcatcggtgttcgaccgcctcaccgacatccgcgagtcgcagtcgcgcgggttggagaatcagttcgcggacctgaagaactcgatggtgatttactcgcgcggcagcactgccacggaggcgatcggcgcgttcagcgacggtttccgtcagctcggcgatgcgacgatcaataccgggcaggcggcgtcattgcgccgttactacgaccggacgttcgccaacaccaccctcgacgacagcggaaaccgcgtcgacgtccgcgcgctcatcccgaaatccaacccccagcgctatctgcaggcgctctataccccgccgtttcagaactgggagaaggcgatcgcgttcgacgacgcgcgcgacggcagcgcctggtcggccgccaatgccagattcaacgagttcttccgcgagatcgtgcaccgcttcaacttcgaggatctgatgctgctcgacctcgagggcaacgtggtgtactccgcctacaaggggccggatctcgggacaaacatcgtcaacggcccctatcgcaaccgggaactgtcggaagcctacgagaaggcggtcgcgtcgaactcgatcgactatgtcggtgtcaccgacttcgggtggtacctgcctgccgaggaaccgaccgcctggttcctgtccccggtcgggttgaaggaccgagtcgacggtgtgatggcggtccagttcccgatcgcgcggatcaacgaattgatgacggcgcggggacagtggcgtgacaccgggatgggagacaccggtgagaccatcctggtcggaccggacaatctgatgcgctcggactcccggctgttccgcgagaaccgggagaagttcctggccgacgtcgtcgaggggggaaccccgccggaggtcgccgacgaatcggttgaccgccgcggcaccacgctggtgcagccggtgaccacccgctccgtcgaggaggcccaacgcggcaacaccgggacgacgatcgaggacgactatctcggccacgaggcgttacaggcgtactcaccggtggacctgccgggactgcactgggtgatcgtggccaagatcgacaccgacgaggcgttcgccccggtggcgcagttcaccaggaccctggtgctgtcgacggtgatcatcatcttcggcgtgtcgctggcggccatgctgctggcgcggttgttcgtccgtccgatccggcggttgcaggccggcgcccagcagatcagcggcggtgactaccgcctcgctctgccggtgttgtctcgtgacgaattcggcgatctgacaacagctttcaacgacatgagtcgcaatctgtcgatcaaggacgagctgctcggcgaggagcgcgccgagaaccaacggctgatgctgtccctgatgcccgaaccggtgatgcagcgctacctcgacggggaggagacgatcgcccaggaccacaagaacgtcacggtgatcttcgccgacatgatgggcctcgacgagttgtcgcgcatgttgacctccgaggaactgatggtggtggtcaacgacctgacccgccagttcgacgccgccgccgagagtctcggggtcgaccacgtgcggacgctgcacgacgggtacctggccagctgcgggttaggcgtgccgcggctggacaacgtccggcgcacggtcaatttcgcgatcgaaatggaccgcatcatcgaccggcacgccgccgagtccgggcacgacctgcggctccgcgcgggcatcgacaccgggtcggcggccagcgggctggtggggcggtccacgttggcgtacgacatgtggggttcggcggtcgatgtcgctaaccaggtgcagcgcggctccccccagcccggcatctacgtcacctcgcgggtgcacgaggtcatgcaggaaactctcgacttcgtcgccgccggggaggtcgtcggcgagcgcggcgtcgagacggtctggcggttgcagggccaccggcgatga

SEQ ID NO: 22 - DNA, 522 bp, Mycobacterium vaccae
acctacgagttcgagaacaaggtcacgggcggccgcatcccgcgcgagtacatcccgtcggtggatgccggcgcgcaggacgccatgcagtacggcgtgctggccggctacccgctggttaacgtcaagctgacgctgctcgacggtgcctaccacgaagtcgactcgtcggaaatggcattcaaggttgccggctcccaggtcatgaagaaggctgccgcccaggcgcagccggtgatcctggagccagtgatggcggtcgaggtcacgacgcccgaggattacatgggtgaagtgagcggcgacctgaactcccgccgtggtcagatccaggccatggaggagcggagcggtgctcgtgtcgtgaaggcgcaggttccgctgtcggagatgttcggctacgtcggagaccttcggtcgaagacccagggccgggccaactactccatggtgttcgactcgtacgccgaagttccggcgaacgtgtcgaaggagatcatcgcgaaggcgacgggccagtaa

SEQ ID NO: 23 - DNA, 570 bp, Mycobacterium vaccae
agacagacagtgatcgacgaaaccctcttccatgccgaggagaagatggagaaggccgtctcggtggcacccgacgacctggcgtcgattcgtaccggccgcgcgaaccccggcatgttcaaccggatcaacatcgactactacggcgcctccaccccgatcacgcagctgtccagcatcaacgtgcccgaggcgcgcatggtggtgatcaagccctacgaggcgagccagctgcgcctcatcgaggatgcgatccgcaactccgacctcggcgtcaatccgaccaacgacggcaacatcatccgggtgtcgatcccgcagctcaccgaggagcgccgccgcgacctggtcaagcaggccaaggccaagggcgaggacgccaaggtgtcggtgcgcaacatccgtcgcaaggcgatggaggaactctcccggatcaagaaggacggcgacgccggcgaagaccaagtgacccgcgccgagaaggatctcgacaagagcacccaccagtacacgaatcagatcgacgaactggtcaagcacaaggaaggcgagttgctggaggtctgacca

SEQ ID NO: 24 - DNA, 1071 bp, Mycobacterium vaccae
cgtggggaaggattgcactctatgagcgaaatcgcccgtccctggcgggttctggcaggtggcatcggtgcctgcgccgcgggtatcgccggggtgctgagcatcgcggtcaccacggcgtcggcccagccgggcctcccgcagcccccgctgcccgcccctgccacagtgacgcaaaccgtcacggttgcgcccaacgccgcgccacaactcatcccgcgccccggtgtgacgcctgccaccggcggcgccgccgcggtgcccgccggggtgagcgccccggcggtcgcgccggcccccgcgctgcccgcccgcccggtgtccacgatcgccccggccacctcgggcacgctcagcgagttcttcgccgccaagggcgtcacgatggagccgcagtccagccgcgacttccgcgccctcaacatcgtgctgccgaagccgcggggctgggagcacatcccggacccgaacgtgccggacgcgttcgcggtgctggccgaccgggtcggcggcaacggcctgtactcgtcgaacgcccaggtggtggtctacaaactcgtcggcgagttcgaccccaaggaagcgatcagccacggcttcgtcgacagccagaagctgccggcgtggcgttccaccgacgcgtcgctggccgacttcggcggaatgccgtcctcgctgatcgagggcacctaccgcgagaacaacatgaagctgaacacgtcccggcgccacgtcattgccaccgcggggcccgaccactacctggtgtcgctgtcggtgaccaccagcgtcgaacaggccgtggccgaagccgcggaggccaccgacgcgattgtcaacggcttcaaggtcagcgttccgggtccgggtccggccgcaccgccacctgcacccggtgcccccggtgtcccgcccgcccccggcgccccggcgctgccgctggccgtcgcaccacccccggctcccgctgttcccgccgtggcgcccgcgccacagctgctgggactgcagggatagacgtcgtcgtcccccgggcgaagcctggcgcccgggggacgacggcccctttct

SEQ ID NO: 25 - DNA, 1364 bp, Mycobacterium vaccae
cgacctccacccgggcgtgaggccaaccactaggctggtcaccagtagtcgacggcacacttcaccgaaaaaatgaggacagaggagacacccgtgacgatccgtgttggtgtgaacggcttcggccgtatcggacgcaacttcttccgcgcgctggacgcgcagaaggccgaaggcaagaacaaggacatcgagatcgtcgcggtcaacgacctcaccgacaacgccacgctggcgcacctgctgaagttcgactcgatcctgggccggctgccctacgacgtgagcctcgaaggcgaggacaccatcgtcgtcggcagcaccaagatcaaggcgctcgaggtcaaggaaggcccggcggcgctgccctggggcgacctgggcgtcgacgtcgtcgtcgagtccaccggcatcttcaccaagcgcgacaaggcccagggccacctcgacgcgggcgccaagaaggtcatcatctccgcgccggccaccgatgaggacatcaccatcgtgctcggcgtcaacgacgacaagtacgacggcagccagaacatcatctccaacgcgtcgtgcaccacgaactgcctcggcccgctggcgaaggtcatcaacgacgagttcggcatcgtcaagggcctgatgaccaccatccacgcctacacccaggtccagaacctgcaggacggcccgcacaaggatctgcgccgggcccgcgccgccgcgctgaacatcgtgccgacctccaccggtgccgccaaggccatcggactggtgctgcccgagctgaagggcaagctcgacggctacgcgctgcgggtgccgatccccaccggctcggtcaccgacctgaccgccgagctgggcaagtcggccaccgtggacgagatcaacgccgcgatgaaggctgcggccgagggcccgctcaagggcatcctcaagtactacgacgccccgatcgtgtccagcgacatcgtcaccgatccgcacagctcgatcttcgactcgggtctgaccaaggtcatcgacaaccaggccaaggtcgtgtcctggtacgacaacgagtggggctactccaaccgcctcgtcgacctggtcgccctggtcggcaagtcgctgtaggggcgagcgaagcgacgggagaacagaggcgccatggcgatcaagtcactcgacgaccttctgtccgaaggggtgacggggcggggcgtactcgtgcgctccgacctgaacgtccccctcgacggcgacacgatcaccgacccggggcgcatcatcgcctcggtgccgacgttgaaggcgttgagtgacgccggcgccaaggtggtcgtcaccgcgcatctgggcaggcccaagggtgagccggatcc

SEQ ID NO: 26 - DNA, 858 bp, Mycobacterium vaccae
gaaatcccgcgtctgaaaccctcttttcgcggcgcccctcaggacggtaagggggccaagcggattgaaaaatgttcgctgaatgagcctgaaattgcgcgtggctcttggaaatcagcagcgatgggtttaccgtgtccactagtcggtccaaagaggaccactggttttcggaggttttgcatgaacaaagcagagctcatcgacgtactcactgagaagctgggctcggatcgtcggcaagcgactgcggcggtggagaacgttgtcgacaccatcgtgcgcgccgtgcacaagggtgagagcgtcaccatcacgggcttcggtgttttcgagcagcgtcgtcgcgcagcacgcgtggcacgcaatccgcgcaccggcgagaccgtgaaggtcaagcccacctcagtcccggcattccgtcccggcgctcagttcaaggctgttgtctctggcgcacagaagcttccggccgagggtccggcggtcaagcgcggtgtgaccgcgacgagcaccgcccgcaaggcagccaagaaggctccggccaagaaggctgccgcgaagaaggccgcgccggccaagaaggctccggcgaagaaggctgcgaccaaggctgcaccggccaagaaggccactgccgccaagaaggccgcgccggccaagaaggccactgccgccaagaaggctgcaccggccaagaaggctccggccaagaaggctgcgaccaaggctgcaccggccaagaaggctccggccaagaaggccgcgaccaaggctgcaccggccaagaaggctccggccgccaagaaggcgcccgccaagaaggctccggccaagcgcggcggacgcaagtaagtc

SEQ ID NO: 27 - protein, 231 aa, Mycobacterium vaccae
DTVLMPPANNRRSSTAGRNLTIMNISMKTLAGAGFAMTAAVGLSLGTAGSAAAAPVGPGCAAYVQQVPDGPGSVQGMASSPVATAAADNPLLTTLSQAISGQLNPNVNLVDTFNGGQFTVFAPTNDAFAKIDPATLETLKTDSDLLTKILTYHVVPGQAAPDQVVGEHVTVEGAPVTVSGMADQLKVNDASVVCGGVQTANATVYLIDTVLMPPAAPGGTTEEGPPHPASP

SEQ ID NO: 28 - protein, 228 aa, Mycobacterium vaccae
MMTTRRKSAAVAGIAAVAILGAAACSSEDGGSTASSASSTASSAMESATDEMTTSSAAPSADPAANLIGSGCAAYAEQVPEGPGSVAGMAADPVTVAASNNPMLQTLSQALSGQLNPQVNLVDTLDGGEFTVFAPTDDAFAKIDPATLETLKTDSDMLTNILTYHVVPGQAAPDQVVGEHVTVEGAPVTVSGMADQLKVNDASVVCGGVQTANATVYLIDTVLMPPAA

SEQ ID NO: 29 - protein, 326 aa, Mycobacterium vaccae
MRLLDRIRGPWARRFGVVAVATAMMPALVGLAGGSATAGAFSRPGLPVEYLMVPSPSMGRDIKIQFQSGGENSPALYLLDGLRAQEDFNGWDINTQAFEWFLDSGISVVMPVGGQSSFYTDWYAPARNKGPTVTYKWETFLTQELPGWLQANRAVKPTGSGPVGLSMAGSAALNLATWHPEQFIYAGSMSGFLNPSEGWWPFLINISMGDAGGFKADDMWGKTEGIPTAVGQRNDPMLNIPTLVANNTRIWVYCGNGQPTELGGGDLPATFLEGLTIRTNETFRDNYIAAGGHNGVFNFPANGTHNWAYWGRELQAMKPDLQAHLL

SEQ ID NO: 30 - protein, 161 aa, Mycobacterium vaccae
SGWDINTAAFEWYVDSGLAVIMPVGGQSSFYSDWYSPACGKAGCQTYKWETFLTQELPAYLAANKGVDPNRNAAVGLSMAGSAALTLAIYHPQQFQYAGSLSGYLNPSEGWWPMLINISMGDAGGYKANDMWGRTEDPSSAWKRNDPMVNIGKLVANNTPL

SEQ ID NO: 31 - protein, 334 aa, Mycobacterium vaccae
MKFTEKWRGSAKAAMHRVGVADMAAVALPGLIGFAGGSATAGAFSRPGLPVEYLDVFSPSMGRDIRVQFQGGGTHAVYLLDGLRAQDDYNGWDINTPAFEWFYESGLSTIMPVGGQSSFYSDWYQPSRGNGQNYTYKWETFLTQELPTWLEANRGVSRTGNAFVGLSMAGSAALTYAIHHPQQFIYASSLSGFLNPSEGWWPMLIGLAMNDAGGFNAESMWGPSSDPAWKRNDPMVNINQLVANNTRIWIYCGTGTPSELDTGTPGQNLMAAQFLEGFTLRTNIAFRDNYIAAGGTNGVFNFPASGTHSWGYWGQQLQQMKPDIQRVLGAQATA

SEQ ID NO: 32 - protein, 161 aa, Mycobacterium vaccae
NGWDINTPAFEWFYESGLSTIMPVGGQSSFYSDWYQPSRGNGQNYTYKWETFLTQELPTWLEANRGVSRTGNAFVGLSMAGSAALTYAIHHPQQFIYASSLSGFLNPSEGWWPMLIGLAMNDAGGFNAESMWGPSSDPAWKRNDPMVNINQLVANNTRIWI

SEQ ID NO: 33 - protein, 142 aa, Mycobacterium vaccae
MRTATTKLGAALGAAALVAATGMVSAATANAQEGHQVRYTLTSAGAYEFDLFYLTTQPPSMQAFNADAYAFAKREKVSLAPGVPWVFETTMADPNWAILQVSSTTRGGQAAPNAHCDIAVDGQEVLSQHDDPYNVRCQLGQW

SEQ ID NO: 34 - protein, 285 aa, Mycobacterium vaccae
MQVRRVLGSVGAAVAVSAALWQTGVSIPTASADPCPDIEVIFARGTGAEPGLGWVGDAFVNALRPKVGEQSVGTYAVNYPAGFDFDKSAPMGAADASGRVQWMADNCPDTKLVLGGMSQGAGVIDLITVDPRPLGRFTPTPMPPRVADHVAAVVVFGNPLRDIRGGGPLPQMSGTYGPKSIDLCALDDPFCSPGFNLPAHFAYADNGMVEEAANFARLEPGQSVELPEAPYLHLFVPRGEVTLEDAGPLREGDAVRFTASGGQRVTATAPAEILVWEMHAGLGAA

SEQ ID NO: 35 - protein, 159 aa, Mycobacterium vaccae
MTAGAAAAATLGAAAVGVTSIAVGAGVAGASPAVLNAPLLSAPAPDLQGPLVSTLSALSGPGSFAGAKATYVQGGLGRIEARVADSGYSNAAAKGYFPLSFTVAGIDQNGPIVTANVTAAAPTGAVATQPLTFIAGPSPTGWQLSKQSALALMSAVIAA

SEQ ID NO: 36 - protein, 166 aa, Mycobacterium vaccae
MPVRRARSALASVTFVAAACVGAEGTALAATPDWSGRYTVVTFASDKLGTSVAARQPEPDFSGQYTFSTSCVGTCVATASDGPAPSNPTIPQPARYTWDGRQWVFNYNWQWECFRGADVPREYAAARSLVFYAPTADGSMFGTWRTDILDGLCKGTVIMPVAAYPA

SEQ ID NO: 37 - protein, 136 aa, Mycobacterium vaccae
MKFTGMTVRASRRALAGVGAACLFGGVAAATVAAQMAGAQPAECNASSLTGTVSSVTGQARQYLDTHPGANQAVTAAMNQPRPEAEANLRGYFTANPAEYYDLRGILAPIGDAQRNCNITVLPVELQTAYDTFMAG

SEQ ID NO: 38 - protein, 376 aa, Mycobacterium vaccae
VIEIDHVTKRFGDYLAVADADFSIAPGEFFSMLGPSGCGKTTTLRMIAGFETPTEGAIRLEGADVSRTPPNKRNVNTVFQHYALFPHMTVWDNVAYGPRSKKLGKGEVRKRVDELLEIVRLTEFAERRPAQLSGGQQQRVALARALVNYPSALLLDEPLGALDLKLRHVMQFELKRIQREVGITFIYVTHDQEEALTMSDRIAVMNAGNVEQIGSPTEIYDRPATVFVASFIGQANLWAGRCTGRSNRDYVEIDVLGSTLKARPGETTIEPGGHATLMVRPERIRVTPGSQDAPTGDVACVRATVTDLTFQGPVVRLSLAAPDDSTVIAHVGPEQDLPLLRPGDDVYVSWAPEASLVLPGDDIPTTEDLEEMLDDS

SEQ ID NO: 39 - protein, 348 aa, Mycobacterium vaccae
SDSGTSSTTSQDSGPASGALRVSNWPLYMADGFIAAFQTASGITVDYKEDFNDNEQWFAKVKEPLSRKQDIGADLVIPTEFMAARVKGLGWLNEISEAGVPNRKNLRQDLLDSSIDEGRKFTAPYMTGMVGLAYNKAATGRDIRTIDDLWDPAFKGRVSLFSDVQDGLGMIMLSQGNSPENPTTESIQQAVDLVREQNDRGQIRRFTGNDYADDLAAGNIAIAQAYSGDVVQLQADNPDLQFIVPESGGDWFVDTMVIPYTTQNQKAAEAWIDYIYDRANYAKLVAFTQFVPALSDMTDELAKVDPASAENPLINPSAEVQANLKSWAALTDEQTQEFNTAYAAVTGG

SEQ ID NO: 40 - protein, 541 aa, Mycobacterium vaccae
MAKTIAYDEEARRGLERGLNALADAVKVTLGPKGRNVVLEKKWGAPTITNDGVSIAKEIELEDPYEKIGAELVKEVAKKTDDVAGDGTTTATVLAQALVREGLRNVAAGANPLGLKRGIEKAVEAVTQSLLKSAKEVETKEQISATAAISAGDTQIGELIAEAMDKVGNEGVITVEESNTFGLQLELTEGMRFDKGYISGYFVTDAERQEAVLEDPYILLVSSKVSTVKDLLPLLEKVIQAGKPLLIIAEDVEGEALSTLVVNKIRGTFKSVAVKAPGFGDRRKAMLQDMAILTGGQVVSERVGLSLETADVSLLGQARKVVVTKDETTIVEGSGDSDAIAGRVAQIRAEIENSDSDYDREKLQERLAKLAGGVAVIKAGAATEVELKERKHRIEDAVRNAKAAVEEGIVAGGGVALLQSAPALDDLGLTGDEATGANIVRVALSAPLKQIAFNGGLEPGVVAEKVSNLPAGHGLNAATGEYEDLLKAGVADPVKVTRSALQNAASIAALFLTTEAVVADKPEKASAPAGDPTGGMGGMDF

SEQ ID NO: 41 - protein, 215 aa, Mycobacterium vaccae
MAKTIAYDEEARRGLERGLNALADAVKVTLGPKGRNVVLEKKWGAPTITNDGVSIAKEIELEDPYEKIGAELVKEVAKKTDDVAGDGTTTATVLAQALVREGLRNVAAGANPLGLKRGIEKAVEAVTQSLLKSAKEVETKEQISATAAISAGDTQIGELIAEAMDKVGNEGVITVEESNTFGLQLELTEGMRFDKGYISGYFVTDAERQEAVLED

SEQ ID NO: 42 - protein, 327 aa, Mycobacterium vaccae
DPYILLVSSKVSTVKDLLPLLEKVIQAGKPLLIIAEDVEGEALSTLVVNKIRGTFKSVAVKAPGFGDRRKAMLQDMAILTGGQVVSERVGLSLETADVSLLGQARKVVVTKDETTIVEGSGDSDAIAGRVAQIRAEIENSDSDYDREKLQERLAKLAGGVAVIKAGAATEVELKERKHRIEDAVRNAKAAVEEGIVAGGGVALLQSAPALDDLGLTGDEATGANIVRVALSAPLKQIAFNGGLEPGVVAEKVSNLPAGHGLNAATGEYEDLLKAGVADPVKVTRSALQNAASIAALFLTTEAVVADKPEKASAPAGDPTGGMGGMDF

SEQ ID NO: 43 - protein, 243 aa, Mycobacterium vaccae
DPRHRLVTTKYNPARTWTAENSVGIGGAYLCIYGMEGPGGYQFVGRTTQVWSRYRHTAPFEPGSPWLLRFFDRISWYPVSAEELLELRADMAAGRGSVDITDGVFSLAEHERFLADNADDIAAFRSRQAAAFSAERTAWAAAGEFDRAEKAASKATDADTGDLVLYDGDERVDAPFASSVWKVDVAVGDRVVAGQPLLALEAMKMETVLRAPADGVVTQILVSAGHLVDPGTPLVVVGTGVRA

SEQ ID NO: 44 - protein, 370 aa, Mycobacterium vaccae
MVRAALRYGFGTASLLAGGFVLRALQGTPAALGATPGEVAPVARRSPNYRDGKFVNLEPPSGITMDRDLQRMLLRDLANAASQGKPPGPIPLAEPPKGDPTPAPAAASWYGHSSVLIEVDGYRVLADPVWSNRCSPSRAVGPQRMHDVPVPLEALPAVDAVVISHDHYDHLDIDTIVALAHTQRAPFVVPLGIGAHLRKWGVPEARIVELDWHEAHRIDDLTLVCTPARHFSGRLFSRDSTLWASWVVTGSSHKAFFGGDTGYTKSFAEIGDEYGPFDLTLLPIGAYHPAFADIHMNPEEAVRAHLDLTEVDNSLMVPIHWATFRLAPHPWSEPAERLLTAADAERVRLTVPIPGQRVDPESTFDPWWRF

SEQ ID NO: 45 - protein, 336 aa, Mycobacterium vaccae
MKANHSGCYKSAGPIWSHPSPLCSPALAPSHAGLDNELSLGVHGQGPEHLTIQQWDTFLNGVFPLDRNRLTREWFHSGKATYVVAGEGADEFEGTLELGYHVGFPWSLGVGINFSYTTPNITYDGYGLNFADPLLGFGDSIVTPPLFPGVSITADLGNGPGIQEVATFSVDVAGPGGSVVVSNAHGTVTGAAGGVLLRPFARLISSTGDSVTTYGAPLKHELTTSRWRPPGVNRGPLHAGREAPEVRSKWPTAANACARDSSSLTQGLVVVECHPVTPPHRPRRDGRGSGVWAPALGTYGGDRRRDVTSVAVFAGNPDGPAESPHPSSEPGGSKEF

SEQ ID NO: 46 - protein, 297 aa, Mycobacterium vaccae
  VARIANT (1)...(297): Xaa = Any Amino Acid
EQPFRLGDWITVPTAAGRPSAHGRVVEVNWRATHIDTGGNLLVMPNAELAGASFTNYSRPVGEHRLTVVTTFNAADTPDDVCEMLSSVAASLPELRTDGQIATLYLGAAEYEKSIPLHTPAVDDSVRSTYLRWVWYAARRQELRXNGVADXFDTPERIASAMRAVASTLRLADDEQQEIADVVRLVRYGNGERLQQPGQVPTGMRFIVDGRVSLSVIDQDGDVIPARVLERGDFLGQTTLTREPVLATAHALEEVTVLEMARDEIERLVHRKPILLHVIGAVADRRAHELRLMDSQD

SEQ ID NO: 47 - protein, 670 aa, Mycobacterium vaccae
GYQSGRSSLRASVFDRLTDIRESQSRGLENQFADLKNSMVIYSRGSTATEAIGAFSDGFRQLGDATINTGQAASLRRYYDRTFANTTLDDSGNRVDVRALIPKSNPQRYLQALYTPPFQNWEKAIAFDDARDGSAWSAANARFNEFFREIVHRFNFEDLMLLDLEGNVVYSAYKGPDLGTNIVNGPYRNRELSEAYEKAVASNSIDYVGVTDFGWYLPAEEPTAWFLSPVGLKDRVDGVMAVQFPIARINELMTARGQWRDTGMGDTGETILVGPDNLMRSDSRLFRENREKFLADVVEGGTPPEVADESVDRRGTTLVQPVTTRSVEEAQRGNTGTTIEDDYLGHEALQAYSPVDLPGLHWVIVAKIDTDEAFAPVAQFTRTLVLSTVIIIFGVSLAAMLLARLFVRPIRRLQAGAQQISGGDYRLALPVLSRDEFGDLTTAFNDMSRNLSIKDELLGEERAENQRLMLSLMPEPVMQRYLDGEETIAQDHKNVTVIFADMMGLDELSRMLTSEELMVVVNDLTRQFDAAAESLGVDHVRTLHDGYLASCGLGVPRLDNVRRTVNFAIEMDRIIDRHAAESGHDLRLRAGIDTGSAASGLVGRSTLAYDMWGSAVDVANQVQRGSPQPGIYVTSRVHEVMQETLDFVAAGEVVGERGVETVWRLQGHRR

SEQ ID NO: 48 - protein, 173 aa, Mycobacterium vaccae
TYEFENKVTGGRIPREYIPSVDAGAQDAMQYGVLAGYPLVNVKLTLLDGAYHEVDSSEMAFKVAGSQVMKKAAAQAQPVILEPVMAVEVTTPEDYMGEVIGDLNSRRGQIQAMEERSGARVVKAQVPLSEMFGYVGDLRSKTQGRANYSMVFDSYAEVPANVSKEIIAKATGQ

SEQ ID NO: 49 - protein, 187 aa, Mycobacterium vaccae
  VARIANT (1)...(187): Xaa = Any Amino Acid
VIDETLFHAEEKMEKAVSVAPDDLASIRTGRANPGMFNRINIDYYGASTPITQLSSINVPEARMVVIKPYEASQLRLIEDAIRNSDLGVNPTNDGNIIRVSIPQLTEERRRDLVKQAKAKGEDAKVSVRNIRRKAMEELSRIKKDGDAGEDEVTRAEKDLDKSTHQYTNQIDELVKHKEGELLEVXP

SEQ ID NO: 50 - protein, 331 aa, Mycobacterium vaccae
MSEIARPWRVLAGGIGACAAGIAGVLSIAVTTASAQPGLPQPPLPAPATVTQTVTVAPNAAPQLIPRPGVTPATGGAAAVPAGVSAPAVAPAPALPARPVSTIAPATSGTLSEFFAAKGVTMEPQSSRDFRALNIVLPKPRGWEHIPDPNVPDAFAVLADRVGGNGLYSSNAQVVVYKLVGEFDPKEAISHGFVDSQKLPAWRSTDASLADFGGMPSSLIEGTYRENNMKLNTSRRHVIATAGPDHYLVSLSVTTSVEQAVAEAAEATDAIVNGFKVSVPGPGPAAPPPAPGAPGVPPAPGAPALPLAVAPPPAPAVPAVAPAPQLLGLQG

SEQ ID NO: 51 - protein, 340 aa, Mycobacterium vaccae
VTIRVGVNGFGRIGRNFFRALDAQKAEGKNKDIEIVAVNDLTDNATLAHLLKFDSILGRLPYDVSLEGEDTIVVGSTKIKALEVKEGPAALPWGDLGVDVVVESTGIFTKRDKAQGHLDAGAKKVIISAPATDEDITIVLGVNDDKYDGSQNIISNASCTTNCLGPLAKVINDEFGIVKGLMTTIHAYTQVQNLQDGPHKDLRRARAAALNIVPTSTGAAKAIGLVLPELKGKLDGYALRVPIPTGSVTDLTAELGKSATVDEINAAMKAAAEGPLKGILKYYDAPIVSSDIVTDPHSSIFDSGLTKVIDNQAKVVSWYDNEWGYSNRLVDLVALVGKSL

SEQ ID NO: 52 - protein, 223 aa, Mycobacterium vaccae
MNKAELIDVLTEKLGSDRRQATAAVENVVDTIVRAVHKGESVTITGFGVFEQRRRAARVARNPRTGETVKVKPTSVPAFRPGAQFKAVVSGAQKLPAEGPAVKRGVTATSTARKAAKKAPAKKAAAKKAAPAKKAPAKKAATKAAPAKKATAAKKAAPAKKATAAKKAAPAKKAPAKKAATKAAPAKKAPAKKAATKAAPAKKAPAAKKAPAKKAPAKRGGRK